(19) United States
(12) Patent Application Publication
(10) Pub. No.: US 2009/0149480 A1
(43) Pub. Date: Jun. 11, 2009

(54) METHODS OF USING PDE V INHIBITORS FOR THE TREATMENT OF CONGESTIVE HEART FAILURE

(75) Inventors: Cynthia Cuffie-Jackson, Far Hills, NJ (US); Enrico P. Veltri, Princeton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/364,305

(22) Filed: Feb. 2, 2009

Related U.S. Application Data
(63) Continuation of application No. 11/280,909, filed on Nov. 16, 2005, now abandoned.
(60) Provisional application No. 60/629,030, filed on Nov. 18, 2004.

(51) Int. Cl. A61K 31/522

(52) U.S. Cl. 514/263.22; 514/263.36

(57) ABSTRACT

The uses of PDE V inhibitors in methods for the treatment of congestive heart failure and other physiological disorders, as a monotherapy and in combination with other active agents are disclosed. Such PDE V inhibitors include those having the formula (I), with the variables defined herein:

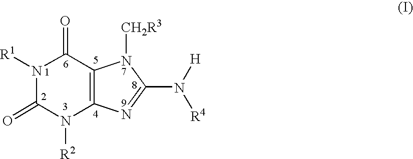

For example, a representative compound useful in the methods of the invention is:

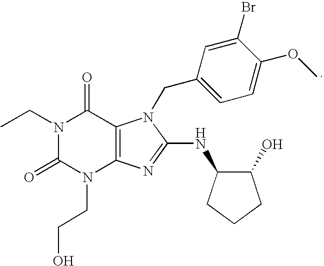

### CROSS-REFERENCE TO RELATED PATENT APPLICATION

The present application claims priority under 35 USC section 119(e) to U.S. Provisional application Ser. No. 60/629,030, filed Nov. 18, 2004, which is incorporated by reference herein as if fully set forth.

### BACKGROUND OF THE INVENTION

1. Field of the Invention

The present invention relates to novel methods for treating congestive heart failure (“CHF”) in mammals, especially humans, with a compound which inhibits phosphodiesterase type V (“PDE V”).

The present invention also relates to pharmaceutical compositions for the treatment of CHF comprising a compound which inhibits PDE type V.

2. Description of Related Art

CHF is a disorder in which the heart loses its ability to pump blood efficiently. The prevalence of CHF is about 1-2% of the general population. In the US, more than three million people have CHF, and more than 400,000 new patients present yearly. Approximately 30-40% of patients with CHF are hospitalized every year. CHF is the leading diagnosis-related group among hospitalized patients older than 65 years. The 5-year mortality rate after diagnosis was reported in 1971 as 60% in men and 45% in women. In 1991, data from the Framingham heart study showed the 5-year mortality rate for CHF essentially remaining unchanged, with a median survival of 3.2 years for males and 5.4 years for females. This may be secondary to an aging US population with declining mortality due to other diseases.

CHF may be caused by the occurrence of an index event such as a myocardial infarction (heart attack) or be secondary to other causes such as hypertension or cardiac malformations such as valvular disease. The index event or other causes result in an initial decline in the pumping capacity of the heart, for example by damaging the heart muscle. This decline in pumping capacity may not be immediately noticeable, due to the activation of one or more compensatory mechanisms. However, the progression of CHF has been found to be independent of the patient's hemodynamic status. Therefore, the damaging changes caused by the disease are present and ongoing even while the patient remains asymptomatic. In fact, the compensatory mechanisms which maintain normal cardiovascular function during the early phases of CHF may actually contribute to progression of the disease, for example by exerting deleterious effects on the heart and circulation.

Some of the more important pathophysiologic changes which occur in CHF are activation of the hypothalamic-pituitary-adrenal axis, systemic endothelial dysfunction and myocardial remodeling.

Therapies specifically directed at counteracting the activation of the hypothalamic-pituitary-adrenal axis include beta-adrenergic blocking agents (beta-blockers), angiotensin converting enzyme (ACE) inhibitors, certain calcium channel blockers, nitrates and endothelin-1 blocking agents. Calcium channel blockers and nitrates, while producing clinical improvement, have not been clearly shown to prolong survival, whereas beta-blockers and ACE inhibitors have been shown to significantly prolong life, as have aldosterone antagonists. Experimental studies using endothelin-1 blocking agents have shown a beneficial effect.

Current therapy for heart failure is insufficient. Although angiotensin converting enzyme (ACE) inhibitors have been shown to have beneficial effects in patients with heart failure, they appear consistently unable to relieve symptoms in more than 60% of heart failure patients. In addition, they reduce mortality of heart failure only by approximately 15-20%. Therefore, there is room for improvement in the therapy of heart failure.

The role of cGMP and PDE V inhibitors has recently been explored as potential treatment for CHF. Preclinical studies in a mice model of CHF (Takimoto, E. et al, Nat. Med. vol. 11, no. 2, 214-222, February 2005) have demonstrated that chronic inhibition of cGMP PDE V prevents and also reverses cardiac hypertrophy in mice. Acute administration of a PDE V inhibitor improved cardiac hemodynamics in the cardiomyopathic hamster model of heart failure (Inoue, H. et al, Eur. J. of Pharmacology, 443,179-184, 2002). Chronic treatment of these hamsters with PDE V inhibitors has been demonstrated to improve survival rates (Inoue et al, 2002). The data in the dog pacing induced model of heart failure is mixed with one study showing some benefit (Yamamoto, T. et al, Clin. Sci., Supp. 48, 258S-262S, 2002), and another showing none (Chen, Y., et al, Am. J. Physiol Heart Circ. Physiol., 284, H1513-H1520, 2003). Beneficial effects of PDE V inhibition on renal function have been reported in animal models of heart failure. The relevance of these animal models, especially in mice and rats, has been questionable. Studies in humans with coronary artery diseases and heart failure have demonstrated modest reductions in blood pressure, peripheral vasodilation, but no effects on cardiac contractility or cardiac output. However, no long term studies in humans have been reported. A recent study concludes that the increase in cGMP caused by sildenafil inhibits cardiac hypertrophy (Mendelsohn, M., Nat. Med., 11, 115-116, February 2002). The potential beneficial effects of PDE V inhibition in CHF could result from reduction in pre-load and after-load, improved renal function and possibly from cardiac remodeling. It is unlikely that PDE V inhibition would have direct effects on cardiac contractility. Any effects on cardiac function may be secondary to its effects on cardiac hypertrophy and remodeling.

PDE V inhibitor compounds and their use in treating a variety of physiological conditions are described in a number of patents (e.g., U.S. Pat. Nos. 5,409,934, 5,470,579, 5,939,419 and 5,393,755) and foreign publications (e.g., WO 93/23401, WO 92/05176, WO 92/05175, and WO 99/24433).

Specific PDE V inhibitors have been found useful for specific indications. For example, the use of PDE V inhibitors for treating impotence has met with commercial success with the introduction of sildenafil citrate, vardenafil, and tadalafil (i.e., Viagra®, Levitra®, and Cialis®, respectively). The chemistry and use of Viagra®, including its mechanism of action in treating erectile dysfunction, are taught in EP 0 702 555 B1.

Accordingly, it is an object of this invention to provide a method of using a PDE V inhibitor to treat a patient who has, or is at risk of, congestive heart failure, and/or other cardiovascular conditions.

### DEFINITIONS AND USAGE OF TERMS

The following definitions and terms are used herein or are otherwise known to a skilled artisan. Except where stated otherwise, the following definitions apply throughout the specification and claims. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “alkoxy,” etc.

The term “chemically-compatible,” as used herein, means that a substituent or variable in a structure, process or the like is selected to be capable of resulting in a stable compound.

The term “substituted” or the phrase “with . . . one or more substituents,” as used herein, means the replacement of one or more atoms or radicals, usually hydrogen atoms, in a given structure with a chemically-compatible atom(s) or radical(s) selected from a specified group. In the situations where more than one atom or radical may be replaced with substituents selected from the same specified group, the substituents may be, unless otherwise specified, either the same or different at every position. Radicals of specified groups, such as alkyl, cycloalkyl, alkenyl, cycloalkenyl, alkynyl, arylalkyl, alkylaryl, heterocycloalkyl, aryl and heteroaryl groups, independently of or together with one another, may be substituents for any substituted group, unless otherwise known, stated or shown to be to the contrary.

Representative substituents for alkyl, cycloalkyl, alkenyl, cycloalkenyl, alkynyl, arylalkyl, alkylaryl, aryl, heteroaryl and heterocycloalkyl groups include, but are not limited to, the following moieties: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo (e.g., —Cl and —Br), nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ and —N(R⁵²)CON(R⁵⁰R⁵¹), where:

R⁵⁰, R⁵¹ and R⁵² may be independently selected from the following: a hydrogen atom and a branched or straight-chain, C₁₋₆ alkyl, C₃₋₆ cycloalkyl, C₄₋₆ heterocycloalkyl, heteroaryl and aryl group, with or without substituents. When permissible, R⁵⁰ and R⁵¹ can be joined together to form a carbocyclic or heterocyclic ring system. R⁵⁰, R⁵¹ and R⁵² may also include:

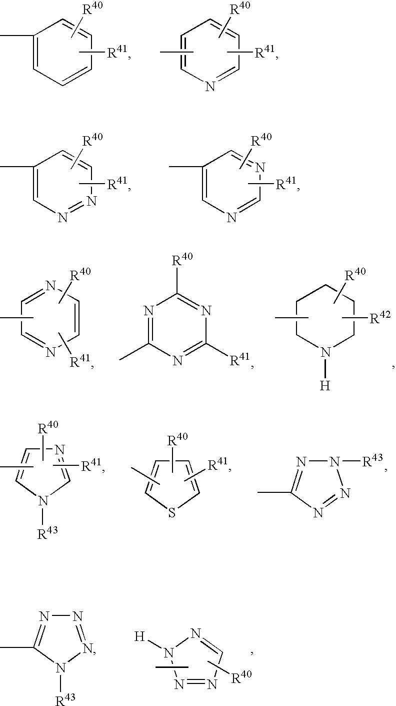

where,
R⁴⁰ and R⁴¹ are, independently of one another, each a hydrogen atom or a branched or straight-chain, optionally substituted, alkyl, cycloalkyl, heterocycloalkyl, halo, aryl, imidazolylalkyl, indolylalkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are as defined above;
R⁴² is a hydrogen atom or a branched or straight-chain, optionally substituted, alkyl, alkenyl, arylalkyl or acyl group; and
R⁴³ is a hydrogen atom or a branched or straight-chain, optionally substituted, alkyl or aryl group;
wherein, the optional substituents are defined the same as above for the one or more substituents.

Preferred substituents on aryl and heteroaryl groups include, but are not limited to, any of the moieties recited above in the definition for R⁴⁰ and R⁴¹.

The term “heteroatom,” as used herein, means a nitrogen, sulfur, or oxygen atom. Multiple heteroatoms in the same group may be the same or different.

The term “hydrocarbon,” as used herein, means a compound or radical consisting of only carbon and hydrogen atoms, including aliphatic, aromatic, normal, saturated and unsaturated hydrocarbons.

The term “alkyl,” as used herein, means an unsubstituted or substituted, straight or branched, hydrocarbon chain (i.e., comprising carbon and hydrogen atoms bonded together), having, preferably, from one to twenty-four carbon atoms, more preferably, from one to twelve carbon atoms, and most preferably, from one to eight carbon atoms.

The term “cycloalkyl” or “cycloalkane,” as used herein, means an unsubstituted or substituted, saturated, stable non-aromatic carbocyclic ring, having, preferably, from three to fifteen carbon atoms, more preferably, from three to eight carbon atoms. The carbon ring radical is saturated and may be fused, for example, benzofused, with one to three cycloalkyl, aromatic, heterocyclic or heteroaromatic rings. The cycloalkyl may be attached at any endocyclic carbon atom that results in a stable structure. Preferred carbocycles have from five to six carbons. Examples of carbocycle radicals include cyclopropyl, cyclobutyl, cyclopentyl, cyclohexyl, cycloheptyl and the like.

The term “alkenyl,” as used herein, means an unsubstituted or substituted, unsaturated, straight or branched, hydrocarbon chain having at least one double bond present and, preferably, from two to fifteen carbon atoms, more preferably, from two to twelve carbon atoms.

The term “cycloalkenyl,” as used herein, means an unsubstituted or substituted, unsaturated carbocyclic ring having at least one double bond present and, preferably, from three to fifteen carbon atoms, more preferably, from five to eight carbon atoms. A cycloalkenyl group is an unsaturated carbocyclic group. Examples of cycloalkenyl groups include cyclopentenyl and cyclohexenyl.

The term “alkynyl,” as used herein, means an unsubstituted or substituted, unsaturated, straight or branched, hydrocarbon chain having at least one triple bond present and, preferably, from two to twelve carbon atoms, more preferably, two to ten carbon atoms.

The term “bicycloalkyl,” as used herein, represents a saturated linearly fused or bridged carbocyclic ring having, preferably, from 5 to 12 carbon atoms.

The term “aryl,” as used herein, means a substituted or unsubstituted, aromatic, mono- or bicyclic carbocyclic ring system having from one to two aromatic rings. The aryl moiety will generally have from 6 to 14 carbon atoms is with all available substitutable carbon atoms of the aryl moiety being intended as possible points of attachment. Representative examples include phenyl, tolyl, xylyl, cumenyl, naphthyl, tetrahydronaphthyl, indanyl, indenyl and the like. If desired, the carbocyclic moiety can be substituted with from one to five, preferably, one to three moieties, such as mono- through pentahalo, alkyl, trifluoromethyl, phenyl, hydroxy, alkoxy, phenoxy, amino, monoalkylamino, dialkylamino and the like.

The term “heteroaryl,” as used herein, means a mono- or bicyclic ring system containing one or two aromatic rings and at least one nitrogen, oxygen or sulfur atom in an aromatic ring. Heteroaryl groups (including bicyclic heteroaryl groups) can be unsubstituted or substituted with a plurality of substituents, preferably, one to five substituents, more preferably, one, two or three substituents (e.g., mono- through pentahalo, alkyl, trifluoromethyl, phenyl, hydroxy, alkoxy, phenoxy, amino, monoalkylamino, dialkylamino and the like). Typically, a heteroaryl group represents a cyclic group of five or six atoms, or a bicyclic group of nine or ten atoms, at least one of which is carbon, and having at least one oxygen, sulfur or nitrogen atom interrupting a carbocyclic ring having a sufficient number of pi (π) electrons to provide aromatic character. Representative heteroaryl (heteroaromatic) groups are pyridinyl, pyrimidinyl, pyrazinyl, pyridazinyl, furanyl, benzofuranyl, thienyl, benzothienyl, thiazolyl, thiadiazolyl, imidazolyl, pyrazolyl, triazolyl, isothiazolyl, benzothiazolyl, benzoxazolyl, oxazolyl, pyrrolyl, isoxazolyl, 1,3,5-triazinyl and indolyl groups.

The term “arylalkyl,” as used herein, means an alkyl moiety substituted with an optionally substituted, aryl or heteroaryl group. Representative arylalkyl groups include a benzyl group and fused bicyclic systems which contain one aryl group.

The term “alkylaryl,” as used herein, means an aryl or heteroaryl moiety substituted with an optionally substituted, alkyl group. Representative alkylaryl groups include o-, m- and p-linked tolyl and xylyl groups.

Unless otherwise known, stated or shown to be to the contrary, the point of attachment for a multiple term substituent (multiple terms that are combined to identify a single moiety) to a subject structure is through the last named term of the multiple term. For example, an “arylalkyl” substituent attaches to a targeted structure through the “alkyl” portion of the substituent. Conversely, when the substituent is “alkylaryl”, it attaches to a targeted structure through the “aryl” portion of the substituent. Similarly, a cycloalkylalkyl substituent attaches to a targeted through the latter “alkyl” portion of the substituent (e.g., Structure-alkyl-cycloalkyl).

The term “heterocycloalkyl,” as used herein, means an unsubstituted or substituted, saturated cyclic ring system having from three to fifteen members, preferably, from three to eight members, and comprising carbon atoms and at least one heteroatom as part of the ring.

The term “heterocyclic ring” or “heterocycle,” as used herein, means an unsubstituted or substituted, saturated, unsaturated or aromatic ring, comprised of carbon atoms and one or more heteroatoms in the ring. Heterocyclic rings may be monocyclic or polycyclic. Monocyclic rings preferably contain from three to eight atoms, most preferably, five to seven atoms. Polycyclic ring systems consisting of two rings preferably contain from six to sixteen atoms, most preferably, ten to twelve atoms. Polycyclic ring systems consisting of three rings contain, preferably, from thirteen to seventeen atoms, most preferably, fourteen to fifteen atoms. Each heterocyclic ring has at least one hetero atom. Unless otherwise stated, the heteroatoms may be independently selected from the following: nitrogen, sulfur and oxygen atoms.

The term “carbocyclic ring” or “carbocycle,” as used herein, means an unsubstituted or substituted, saturated, unsaturated or aromatic (e.g., aryl), hydrocarbon ring, unless otherwise specifically identified. Carbocycles may be monocyclic or polycyclic. Monocyclic rings preferably contain from three to eight atoms, most preferably, five to seven atoms. Polycyclic rings having two rings preferably contain from six to sixteen atoms, most preferably, ten to twelve atoms, and those having three rings preferably contain from thirteen to seventeen atoms, most preferably, fourteen to fifteen atoms.

The term “alkoxy,” as used herein, means an oxygen atom bonded to a hydrocarbon chain, such as an alkyl or alkenyl group (e.g., —O-alkyl or —O-alkenyl). Representative alkoxy groups include methoxy, ethoxy, and isopropoxy groups.

The term “hydroxyalkyl,” as used herein, means a substituted hydrocarbon chain, preferably, an alkyl group, having at least one hydroxy substituent (i.e., —OH). Additional substituents to the alkyl group may also be present. Representative hydroxyalkyl groups include hydroxymethyl, hydroxyethyl and hydroxypropyl groups.

The term “carboxyalkyl,” as used herein, means a substituted hydrocarbon chain, preferably, a substituted alkyl group, which has a carboxyl substituent (e.g., —COOH) and may also have additional substituents (such as one of the representative substituents identified above for the term “substituted”). Representative carboxyalkyl groups include carboxymethyl (—CH₂CO₂H) and carboxyethyl (—CH₂CH₂CO₂H) groups, and derivatives thereof, such as the corresponding esters.

The term “aminoalkyl,” as used herein, means an alkyl group substituted with an amine moiety (e.g., -alkylNH₂), such as aminomethyl.

The term “alkylamino,” as used herein, means an amino moiety having from one or two alkyl substituents (e.g., —NH-alkyl), such as dimethylamino.

The term “alkenylamino,” as used herein, means an amino moiety having from one or two alkenyl substituents, where the nitrogen atom of the amino group is not attached to the alkene-forming carbon atom (e.g., —NH—CH₂-alkenyl), such as dibutenylamino.

The term “arylamino,” as used herein, means an amine moiety substituted with an aryl group (i.e., —NH-aryl).

The term “alkylimino,” as used herein, means an imino moiety having one alkenyl or two alkyl substituents (e.g., —C═N-alkyl).

The term “oximino,” as used herein, means compounds containing the —C═N—OR⁶⁹ radical, where R⁶⁹ is a hydrogen atom or an alkyl or aryl group.

The term “aroyl,” as used herein, means the radical R—CO—; where R is an aromatic group. Representative aroyls are benzoyl and naphthoyl.

The term “aryloxy,” as used herein, means an oxygen atom having an aryl substituent (e.g., —O-aryl).

The term “ester,” as used herein, means compounds containing a substituted carboxylic acid (e.g., —COO-aryl).

The term “acyl” or “carbonyl,” as used herein, means a carbon to oxygen double bond, (e.g., R—C(═O)—), which can be a radical of a carboxylic acid having the formula alkyl-CO—, aryl-CO—, arylalkyl-CO—, cycloalkyl-CO—, alkylcycloalkyl-CO— or heteroaryl-CO—. Representative acyl groups include acetyl, propionyl, butanoyl and benzoyl groups.

The term “acyloxy,” as used herein, means an oxygen atom having an acyl substituent (e.g., —O-acyl), for example, —O—C(═O)-alkyl.

The term “acylamino,” as used herein, means an amino moiety having an acyl substituent (e.g., —NH-acyl), for example, an amide with the formula —NH—(C═O)-alkyl, a urea with the formula —NH—(C═O)—NH-alkyl or a carbamate with the formula —NH—(C═O)—OR, where R is an alkyl, cycloalkyl, alkenyl, cycloalkenyl, alkynyl, arylalkyl or heterocycloalkyl group.

The term “halo,” “halogen” or “halide,” as used herein, means a chloro, bromo, fluoro or iodo atom radical. Chlorides, bromides and fluorides are preferred halides.

The term “lower hydrocarbon” (e.g., “lower alkyl”), as used herein, means a hydrocarbon chain comprised of from, unless otherwise stated, one to eight carbon atoms, preferably, one to six carbon atoms, and most preferably, one to four carbon atoms.

The term “polyhalo,” as used herein, represents substitution of at least two halo atoms to a group modified by the term “polyhalo.”

The term “aminosulfonyl,” as used herein, represents a group having the formula: —SO₂NR⁷⁹R⁸⁹, where R⁷⁹ and R⁸⁹ are, independently of one another, each a hydrogen atom or a lower alkyl (e.g., from 1 to 6 carbon atoms) or aryl group.

The term “sulfonyl,” as used herein, represents a group having the formula: —S(O)₂—.

When a variable appears more than once in a structural formula, for example, R⁵⁹ for where X is —C(OR⁵⁹)₂—, the identity of each variable appearing more than once may be independently selected from the definition for that variable.

The term “pharmaceutically-acceptable excipients,” as used herein, includes any physiologically inert, pharmacologically inactive material known to one skilled in the art, which is compatible with the physical and chemical characteristics of the particular active ingredient selected for use. Pharmaceutically-acceptable excipients include polymers, resins, plasticizers, fillers, binders, lubricants, glidants, disintegrates, solvents, co-solvents, buffer systems, surfactants, preservatives, sweetening agents, flavoring agents, pharmaceutical grade dyes or pigments, and viscosity agents.

The term “pharmaceutical composition,” as used herein, means a combination of at least one subject compound (e.g., PDE V inhibitor) and at least one pharmaceutically-acceptable excipient.

The term “pharmaceutically-acceptable salt,” as used herein, means a cationic salt formed at an acidic (e.g., carboxyl) group or an anionic salt formed at a basic (e.g., amino) group of the compound. Preferred cationic salts include the alkali-metal salts (e.g., sodium and potassium) and alkaline earth metal salts (e.g., magnesium and calcium). Preferred anionic salts include the halide (e.g., chloride), acetate and phosphate salts.

The phrase “effective amount,” as used herein, means an amount of a compound or composition which is sufficient to significantly and positively modify the symptoms and/or conditions to be treated (e.g., provide a positive clinical response). The phrase “safe and effective amount,” as used herein, means that an “effective amount” must also be safe, that is, an amount that is sufficient to provoke a positive response, yet is small enough to avoid serious side effects (at a reasonable benefit/risk ratio), within the scope of sound medical judgment. The effective amount of an active ingredient for use in a pharmaceutical composition will vary with the particular condition being treated, the severity of the condition, the duration of the treatment, the nature of concurrent therapy, the particular active ingredient being employed, the particular pharmaceutically-acceptable excipients utilized and like factors within the knowledge and expertise of the attending physician.

The phrase “administering [to a patient a safe and effective amount of the subject compound],” as used herein, refers to any mode of introducing any form (e.g., solid, liquid or gas) of the subject compounds in vivo to a patient (e.g., human or mammal). For example, introduction of the subject compound to a patient may be accomplished via oral ingestion (e.g., tablets, capsules, gels, solutions, etc.), adsorption, absorption (e.g., transmucosal sublingual or buccal administration), transdermal applications (e.g., topical applications via patches, lotions, etc.), suppositories, etc.

The term “oral dosage form,” as used herein, means any pharmaceutical composition intended to be systemically administered to an individual by delivering the composition to the gastrointestinal tract of an individual, via the mouth of the individual. For purposes of the invention, the delivered form can be a tablet (coated or non-coated), solution, suspension or capsule (coated or non-coated).

The term “injection,” as used herein, means any pharmaceutical composition intended to be systemically administered to a human or other mammal, via delivery of a solution or emulsion containing the active ingredient, by puncturing the skin of said individual, in order to deliver the solution or emulsion to the circulatory system of the individual either by intravenous, intramuscular, intraperitoneal or subcutaneous injection.

The terms “treating” and “treatment” are understood to include preventing, lowering, stopping, or reversing the progression or severity of the condition or symptoms being treated. As such, the terms “treating” and “treatment” include both medical therapeutic administration in the presence of an existing condition and/or prophylactic administration intended for the prevention of such condition, as appropriate.

Other than as shown in the operating examples or where is otherwise indicated, all numbers used in the specification and claims expressing quantities of ingredients, reaction conditions, and so forth, are understood as being modified in all instances by the term “about.”

### SUMMARY OF THE INVENTION

In one aspect, the invention is directed to a method of treating congestive heart failure comprising administering to a patient in need of such treatment an effective amount of a PDE V inhibitor compound, wherein said compound is a compound of Formula (I), an enantiomer, stereoisomer, rotomer, tautomer or a pharmaceutically acceptable salt thereof:

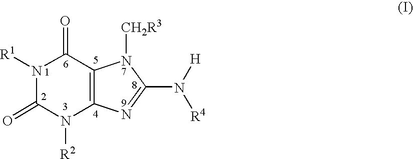

wherein the variables are as defined herein.

In another aspect, the invention is directed to a method of treating congestive heart failure comprising administering to a patient in need of such treatment an effective amount of a PDE V inhibitor compound, wherein said compound is selected from the group consisting of:

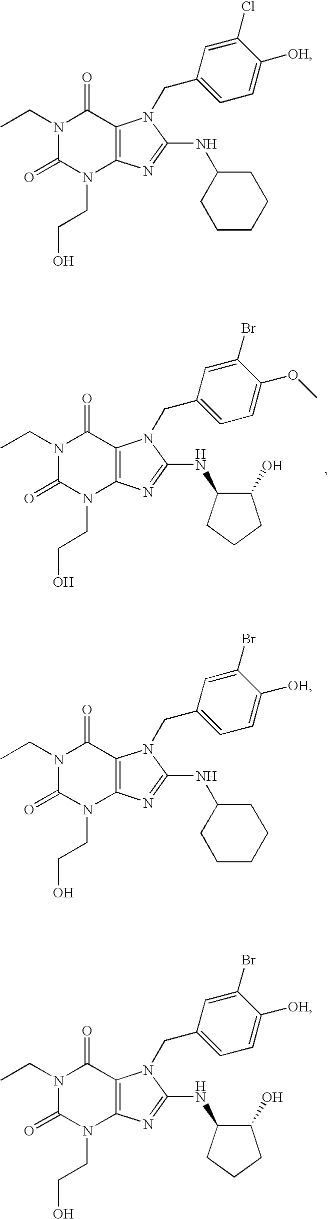

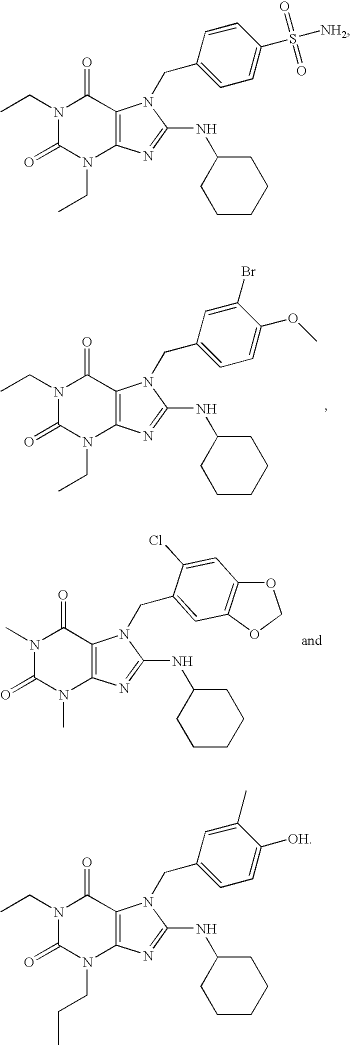

In another aspect, the invention is directed to a method of treating congestive heart failure comprising administering to a patient in need of such treatment an effective amount of a PDE V inhibitor compound, wherein said compound is a compound of the following structure:

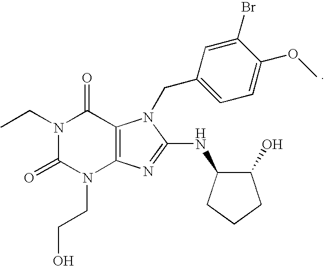

In some embodiments, this method further comprises administering to the patient an effective amount of at least one therapeutic agent selected from the group consisting of prostanoids, α-adrenergic receptor, dopamine receptor agonists, melanocortin receptor agonists, endothelin receptor antagonists, endothelin converting enzyme inhibitors, angiotensin II receptor antagonists, angiotensin converting enzyme inhibitors, neutral metalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5. In some embodiments, the method further comprises administering to the patient an effective amount of at least one ET_{A} receptor antagonist selected from the group consisting of bosentan, atrasentan, ambrisentan, darusentan, sitaxsentan, ABT-627, TBC-3711, CI-1034, SPP-301, SB-234551, ZD-4054, BQ-123 and BE-18257B. In some embodiments, this method further comprises administering to the patient an effective amount of sitaxsentan.

In other embodiments, the invention is directed to a pharmaceutical composition comprising a PDE V inhibitor compound, an ET_{A} receptor antagonist, and a pharmaceutically acceptable carrier. In some embodiments, the PDE V inhibitor compound is selected from the group consisting of those compounds listed in Tables I and II. In some embodiments, the PDE V inhibitor compound is selected from the group consisting of:

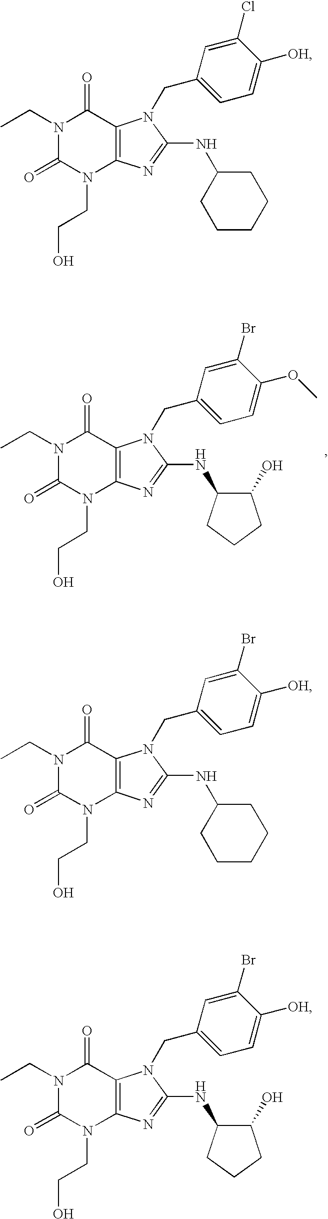

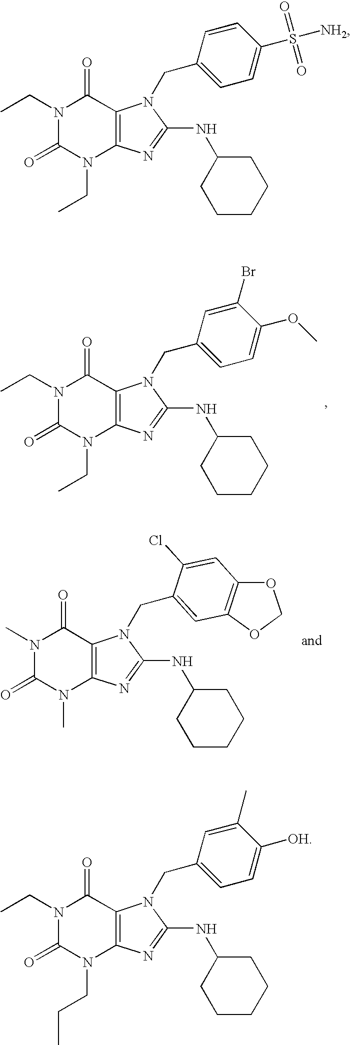

In some embodiments, the PDE V inhibitor compound is

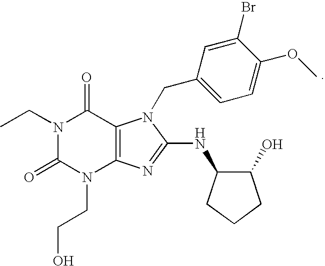

In some embodiments, the ET_{A} receptor antagonist is sitaxsentan.

A further understanding of the invention will be had from the following detailed description of the invention.

### DETAILED DESCRIPTION OF THE INVENTION

Systemic endothelial dysfunction is a well-recognized feature of CHF and is clearly present by the time signs of left ventricular dysfunction are present. Endothelial dysfunction is important with respect to the intimate relationship of the myocardial microcirculation with cardiac myocytes. The evidence suggests that microvascular dysfunction contributes significantly to myocyte dysfunction and the morphological changes which lead to progressive myocardial failure.

Endothelial dysfunction is associated with impairment of aerobic capacity in patients with heart failure. Impaired endothelium-dependent vasodilation in patients with heart failure can be attributed to decreased bioavailability of nitric oxide and attenuated responses to nitric oxide in vascular smooth muscle. Impaired vasodilation in response to nitric oxide derived from vascular endothelium or organic nitrates in vascular smooth muscle may be related in part to increased degradation of the second messenger cyclic guanosine monophosphate by type V phosphodiesterase. Sildenafil, a specific type V phosphodiesterase inhibitor currently approved for the treatment of erectile dysfunction, has been shown to acutely enhance endothelium dependent vasodilation in patients with heart failure. Tadalafil, vardenafil, and sildenafil citrate, which have been similarly approved for the treatment of erectile dysfunction, may also enhance endothelium dependent vasodilation in patients with heart failure. Thus, the use of any PDE V inhibitor (including those of formulas I and II and of Tables I and II, as well as tadalafil, vardenafil, and sildenafil citrate) for the treatment of CHF and/or other cardiovascular conditions is within the scope of the present invention.

The compounds described in U.S. Pub. No. 2002/0169174 (which is herein incorporated in its entirety by reference) are potent PDE V inhibitors, thought to be useful in the treatment of a variety of cardiovascular conditions, including congestive heart failure. The subject compounds having the formula (I) are substituted at the 8-position on the chemical structure with an amino group that itself is substituted with one of the following groups: an unsaturated or saturated carbocyclic group and a saturated heterocyclic group. The substituted xanthines exhibited unexpectedly enhanced properties with respect to enzyme activity and enzyme selectivity. It is believed that the substitution at the 8-position of the subject PDE V inhibitor compounds with these specific groups, helped produce unexpectedly highly potent and selective xanthines, which exhibited increased isozyme selectivity when compared to conventional xanthines. Pharmaceutical compositions comprising the subject compounds possess unexpectedly superior therapeutic properties.

Referring above to the xanthine PDE V inhibitor compounds having the formula (I), the 8-position on the chemical structure is substituted with a —NHR⁴ group, where R⁴ represents a carbocyclic or heterocyclic system defined as follows: a C₃₋₁₅ cycloalkyl group, a C₃₋₁₅ cycloalkenyl group or a heterocycloalkyl group of 3 to 15 members. All of the cyclic systems are optionally substituted. Preferred substituents on the cyclic systems include a C₃₋₆ cycloalkyl group, a C₁₋₆ alkoxy C₁₋₆ alkyl group, a C₁₋₆ alkyl group, an amino C₁₋₆ alkyl group, a C₁₋₆ dialkylamino C₁₋₆ alkyl group, a C₃₋₆ dicycloalkylamino C₁₋₆ alkyl group, a hydroxy group, an alkoxy group, an oximino group, —COR⁶, —SO₂R⁶, —COOR⁶, —CONR⁶R⁷, —SO₂NR⁶R⁷, —N(R⁸)SO₂R⁶ and —NR⁶R⁷, where:
R⁶ is a hydrogen atom or an optionally substituted, C₁₋₆ alkyl, C₃₋₆ cycloalkyl, C₃₋₆ heterocycloalkyl, aryl or heteroaryl group;
R⁷ is a hydrogen atom or an optionally substituted, C₁₋₆ alkyl, C₃₋₆ cycloalkyl, C₃₋₆ heterocycloalkyl, aryl or heteroaryl group; or
R⁶ and R⁷, when applicable, may be joined together to form a heterocyclic ring system; and
R⁸ is a hydrogen atom or an optionally substituted, C₁₋₆ alkyl, C₃₋₆ cycloalkyl, C₃₋₆ heterocycloalkyl, aryl or heteroaryl group.

Furthermore, R⁴ may also be substituted with -ZR⁷⁰Z′—, where R⁷⁰, together with Z and Z′, form a spiro-fused 5- to 7-membered ring or a linearly fused 4- to 7-membered ring system, and Z and Z′, independently of one another, are each an oxygen, sulfur or nitrogen atom. For example, when Z=Z′=O, R⁴ may be substituted by the following structure having the formula (VIII):

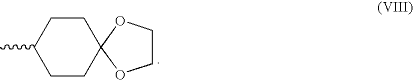

Preferred substituents are defined above for the groups. Other substituents may also be used, such as ketones, oximes, cyclic systems, including lineraly fused and bridged, mono-, bi- and tricyclic rings, spiro-cyclic systems, including ketals and thioketals directly attached to R⁴, halogens and sulfonamides. One skilled in the art can determine other possible substituents depending on the conditions employed and the desired properties.

A preferred structure is represented by formula (II):

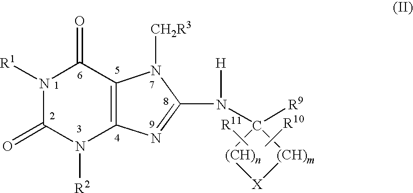

where,
R¹, R² and R³ are defined the same as above for the compound of formula (I);
R⁹ is one of the following atoms or groups:
   (a) a hydrogen atom;
   (b) an oximino group;
   (c) a carboxyalkyl group;
   (d) a C₁₋₆ alkoxy C₁₋₆ alkyl group;
   (e) an aryloxy C₁₋₆ alkyl group;
   (f) a C₃₋₆ cycloalkoxy C₁₋₆ alkyl group;
   (g) a heteroaryloxy C₁₋₆ alkyl group;
   (h) a —COOH group;
   (i) an ester group;
   (j) a C₁₋₆ alkyl group;
   (k) a C₃₋₆ cycloalkyl group;
   (l) a C₃₋₆ heterocyclic group;
   (m) a hydroxy C₁₋₆ alkyl group;
   (n) an aryl group; or
   (o) a heteroaryl group;

wherein, all of the above groups are optionally substituted;
R¹⁰ and R¹¹ are substituents on the same or different carbon atoms of the ring and, independently of one another, are each defined the same as above for R⁹ and, additionally, may each be one of the following groups:
   (a) a hydroxy group;
   (b) an ester group derived from a hydroxy group with a:
      (i) C₁₋₆ carboxylic acid;
      (ii) C₃₋₆ cycloalkyl C₁₋₆ carboxylic acid;
      (iii) aryl C₁₋₆ carboxylic acid; or
      (iv) heteroaryl C₁₋₆ carboxylic acid group;
   (c) a C₁₋₆ alkoxy group;
   (b) an amino group;
   (c) a C₁₋₆ mono- or dialkylamino group;
   (d) a C₁₋₆ alkylacylamino group;
   (e) a C₁₋₆ alkylsulfonylamino group; or
   (f) a —NHCON(R¹⁴)₂ group, where R¹⁴ is a hydrogen
   atom or an optionally substituted, alkyl or aryl group; or
R¹⁰ and R¹¹, taken together with each other and, optionally, with one or more carbon and/or hetero atoms of the ring, form an optionally substituted, spiro-fused, linearly fused, bi- or tri-cyclic ring system of from 8 to 12 members, including from 0 to 4 hetero atoms, where, all of the above R¹⁰, R¹¹ and R¹⁴ groups are optionally substituted;
m and n are, independently of one another, each from 1 to 3; and
X is a chemcially-compatible group, which is —C(R¹⁰R¹¹)—, —S(O)_{y}, —O—, —N(R⁶⁰)—, where:
   R¹⁰ and R¹¹ are, independently of one another, each defined the same as previously;
   y is from 0 to 2;
   R⁶⁰ is a hydrogen atom or a C₁₋₈ alkyl, C₁₋₈ alkynyl, C₁₋₈ alkenyl, C₃₋₈ cycloalkyl, aryl, heteroaryl, C₄₋₈ heterocycloalkyl, COR⁶¹, SO₂R⁶¹, COOR⁶¹, CONR⁶¹R⁶² or SO₂NR⁶¹R⁶² group, with or without substituents, where:
   R⁶¹ is a hydrogen atom or a C₁₋₈ alkyl, C₁₋₈ alkynyl, C₁₋₈ alkenyl, C₃₋₈ cycloalkyl, aryl, heteroaryl or C₄₋₈ heterocyclic group, with or without substituents;
   R⁶² is a hydrogen atom or a C₁₋₈ alkyl, C₁₋₈ alkynyl, C₁₋₈ alkenyl, C₃₋₈ cycloalkyl, aryl, heteroaryl or C₄₋₈ heterocyclic group, with or without substituents; and
   when R⁶¹ and R⁶² are (the same or different) alkyl groups, they can, if desired, be joined together to form a carbocyclic or heterocyclic ring system;
wherein, the optional substituents and the one or more substituents are defined the same as for the one or more substituents of formula (I) above.

In the compound of formula (II), the different carbon atoms to which R¹⁰ and R¹¹ may be connected can be adjacent or non-adjacent. Preferably, R⁹, R¹⁰ and R¹¹ are all hydrogen atoms. In another embodiment of the invention, one of R¹⁰ or R¹¹ is, advantageously, a hydroxy group.

In the compounds of formulas (I) and (II), R¹ is, preferably, an alkyl group or an arylalkyl group, particularly, a benzyl group. More preferably, R¹ is a lower alkyl group of from 1 to 4 carbon atoms, and most preferably, a methyl or ethyl group.

R², in the compounds of formulas (I) and (II), is, preferably, an alkyl group, particularly, an alkyl group substituted with a hydroxy group. More preferably, R² is a lower alkyl group of from 1 to 3 carbon atoms or a hydroxyalkyl group, and most preferably, R² is a methyl, ethyl, iso-butyl or hydroxyethyl group.

In the compounds of formulas (I) and (II), R³ is, preferably, an aryl group, particularly, an aryl group substituted with a hydroxy-, alkoxy- or amino-sulfonyl group, which may be, advantageously, substituted with 1 or 2 halogen atoms. When R³ is a heteroaryl group in the compounds of formulas (I) and (II), it is generally preferable to utilize heteroaryl groups other than furan. Most preferably, R³ is a methoxyaryl group substituted on its aryl ring with at least one halogen atom, for example, a substitution with 1 or 2 halogen atoms, such as chlorine or bromine. For instance, R³ can be 4-hydroxyphenyl, 3-chloro-4-hydroxyphenyl, 3-bromo-4-hydroxyphenyl, 4-methoxyphenyl, 3-chloro-4-methoxyphenyl, 3-bromo-4-methoxyphenyl, 4-aminosulfonylphenyl group, 3-chloro-4-aminosulfonylphenyl group or 3-bromo-4-aminosulfonyl-phenyl.

R⁴, in the compound of formula (I), is, preferably, a cycloalkyl or heterocycloalkyl group, particularly, a cycloalkyl group substituted with a hydroxy group. More preferably, R⁴ is a cyclohexyl, hydroxycyclopentyl or tetrahydropyranyl group. Most preferably, R⁴ is a hydroxycyclopentyl group. For instance, R⁴ can be a 2(R)-hydroxy-1(R)-cyclopentyl group. All of the preferred embodiments may be unsubstituted or substituted.

The following compounds listed in Tables I and II (from U.S. Ser. No. 08/940,760) are illustrative of those compounds used in the inventive methods of treating cardiovascular conditions that include congestive heart failure.

TABLE I

| Compound | |
|---|---|
| No. | Structure |
| 10 | 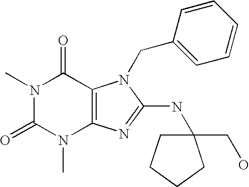 |
| 11 | 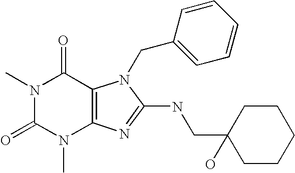 |
| 12 | 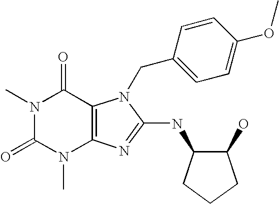 |
| 13 | 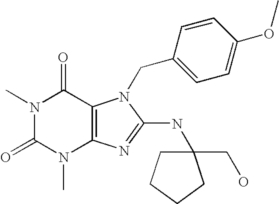 |
| 14 | 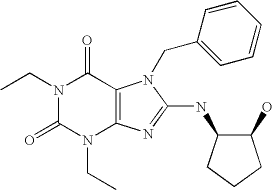 |
| 15 | 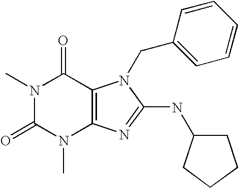 |
| 16 | 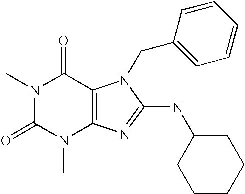 |
| 17 | 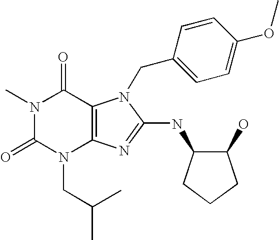 |
| 18 | 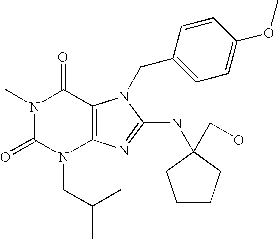 |
| 19 | 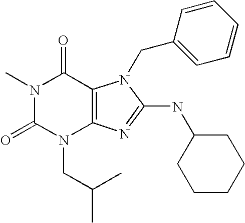 |
| 20 | 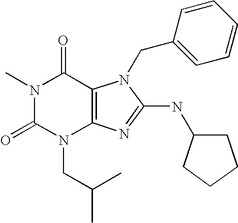 |
| 21 | 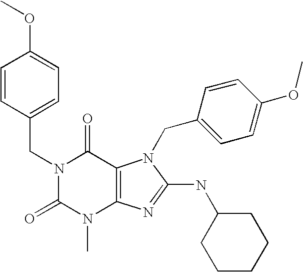 |
| 22 | 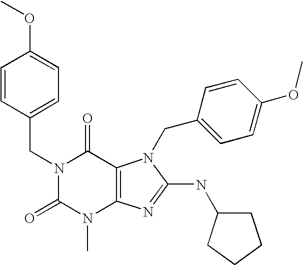 |
| 23 | 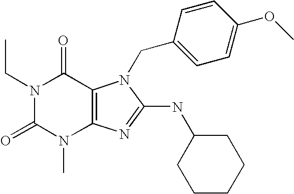 |
| 24 | 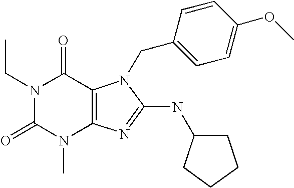 |
| 25 | 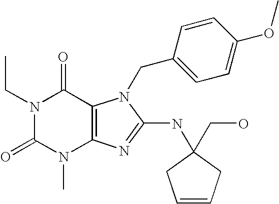 |
| 28 | 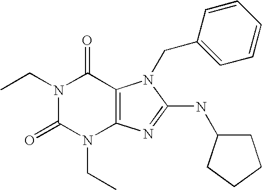 |
| 29 | 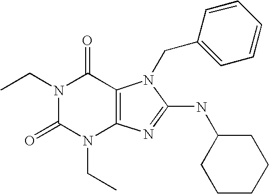 |
| 30 | 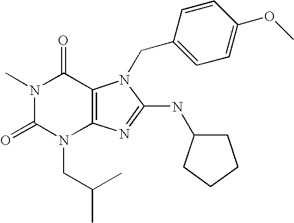 |
| 31 | 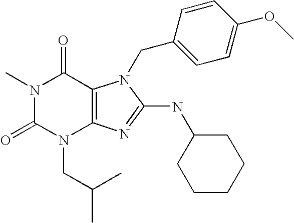 |
| 32 | 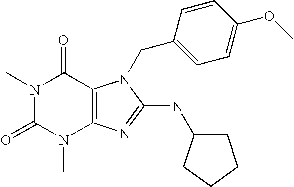 |
| 33 | 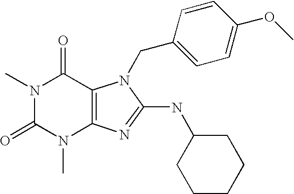 |
| 34 | 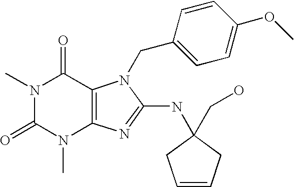 |
| 35 | 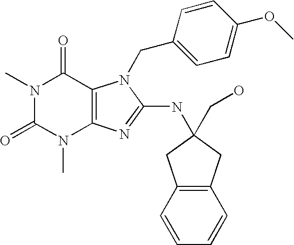 |
| 36 | 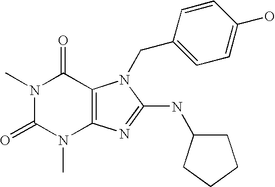 |
| 37 | 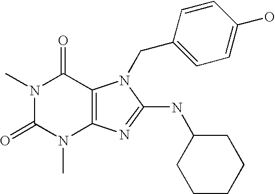 |
| 38 | 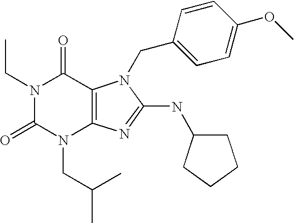 |
| 39 | 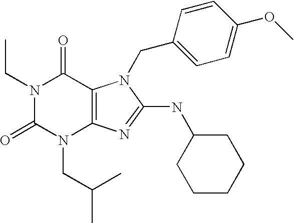 |
| 40 | 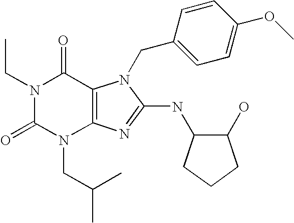 |
| 41 | 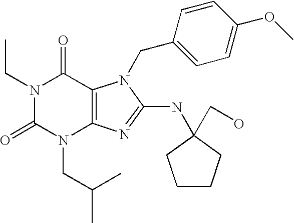 |
| 42 | 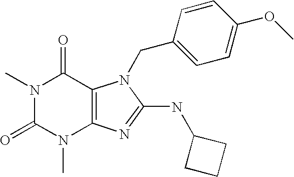 |
| 43 | 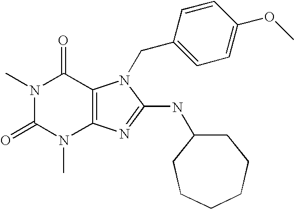 |
| 44 | 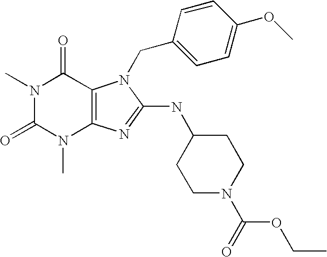 |
| 47 | 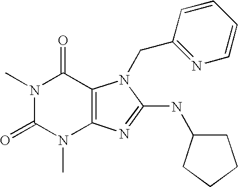 |
| 48 | 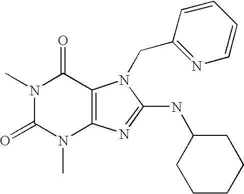 |
| 49 | 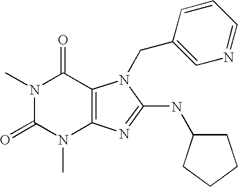 |
| 50 | 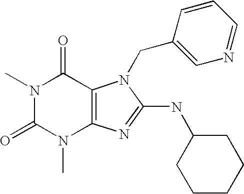 |
| 51 | 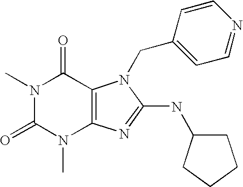 |
| 52 | 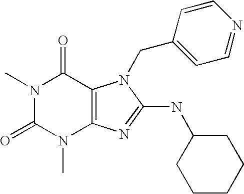 |
| 53 | 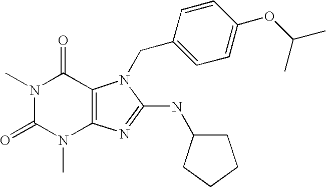 |
| 54 | 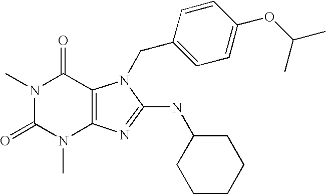 |
| 55 | 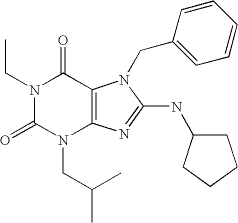 |
| 56 | 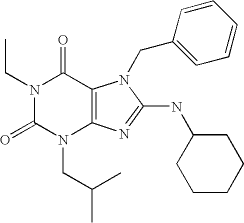 |
| 57 | 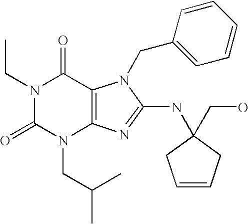 |
| 58 | 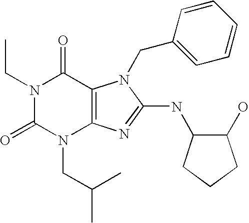 |
| 59 | 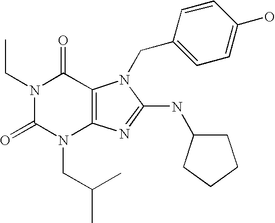 |
| 60 | 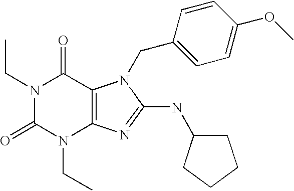 |
| 61 | 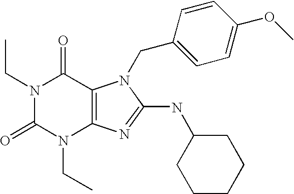 |
| 62 | 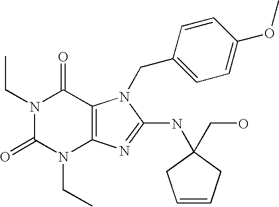 |
| 63 | 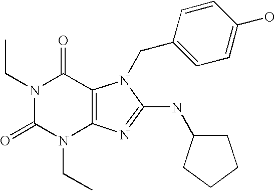 |
| 64 | 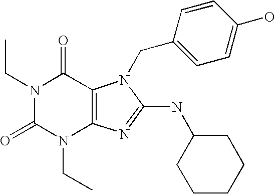 |
| 65 | 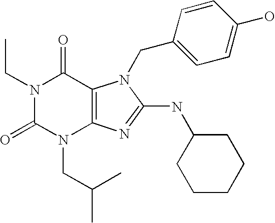 |
| 66 | 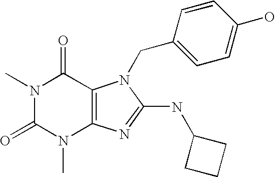 |
| 67 | 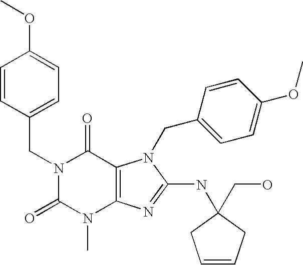 |
| 68 | 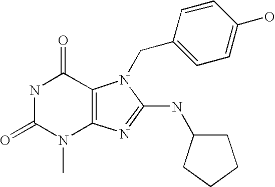 |
| 69 | 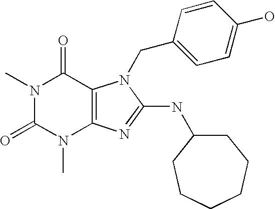 |
| 70 | 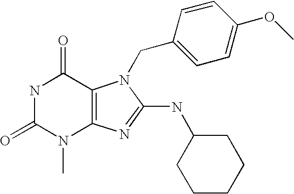 |
| 71 | 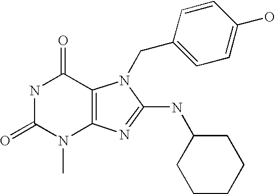 |
| 72 | 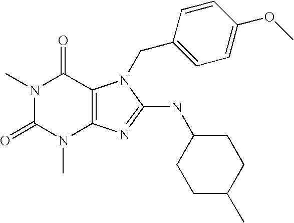 |
| 73 | 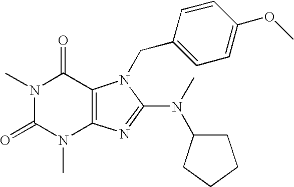 |
| 74 | 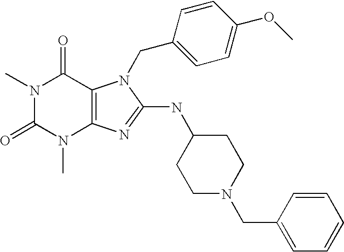 |
| 75 | 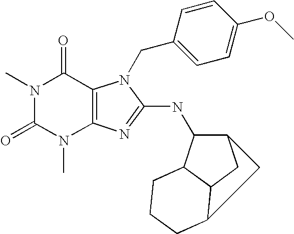 |
| 76 | 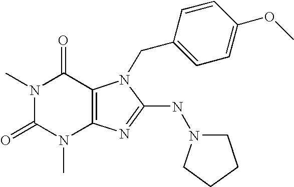 |
| 77 | 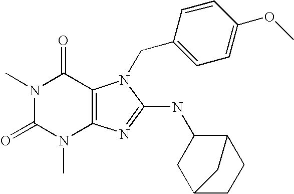 |
| 78 | 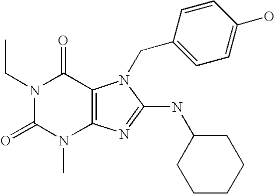 |
| 79 | 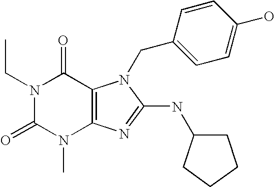 |
| 80 | 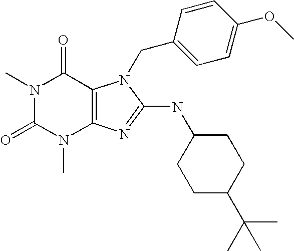 |
| 81 | 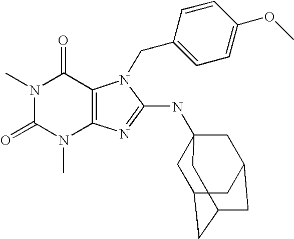 |
| 82 | 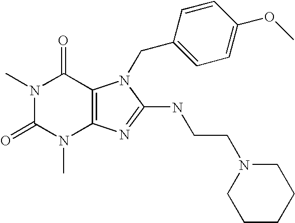 |
| 83 | 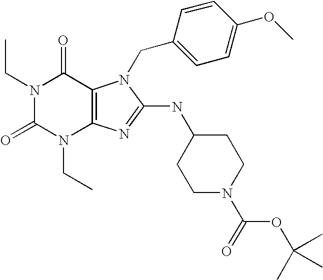 |
| 84 | 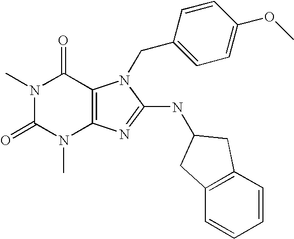 |
| 85 | 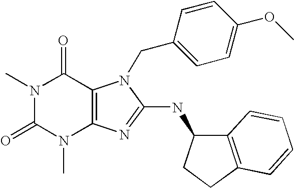 |
| 86 | 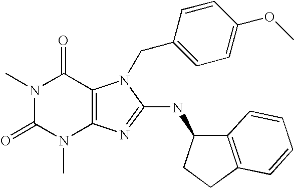 |
| 87 | 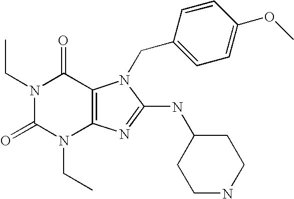 |
| 88 | 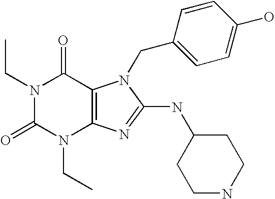 |
| 89 | 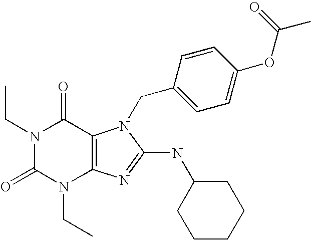 |
| 90 | 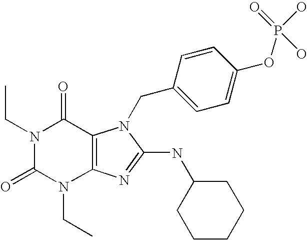 |
| 91 | 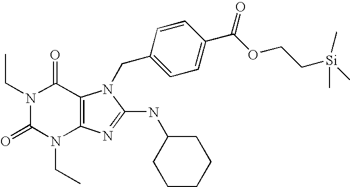 |
| 92 | 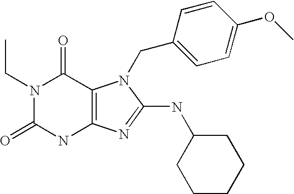 |
| 93 | 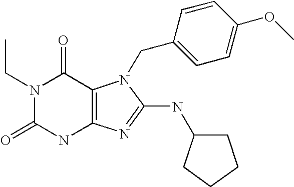 |
| 94 | 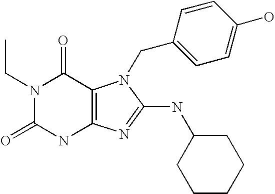 |
| 95 | 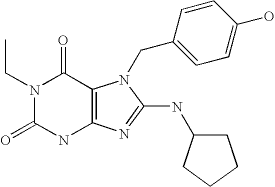 |
| 96 | 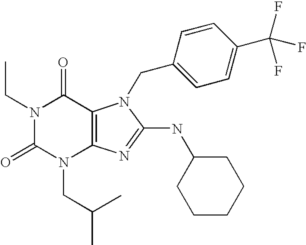 |
| 97 | 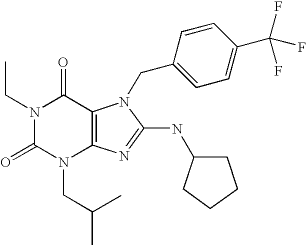 |
| 98 | 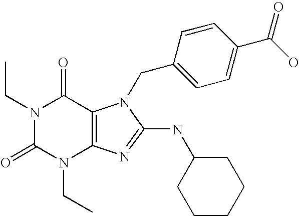 |
| 99 | 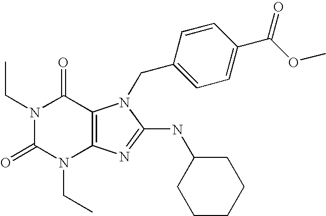 |

TABLE II

| Compound | | HRMS | HRMS | M, |
|---|---|---|---|---|
| No. | STRUCTURE | Calc. | Found | M + 1 |
| 100 | 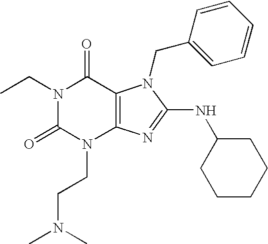 | 439.2821 | 439.2821 | (M + 1) |
| 101 | 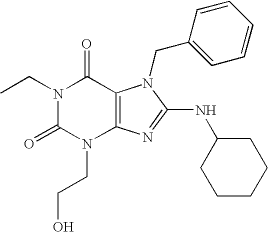 | 412.2349 | 412.2346 | (M + 1) |
| 102 | 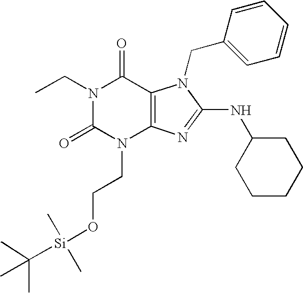 | 526.3213 | 526.3203 | (M+) |
| 103 | 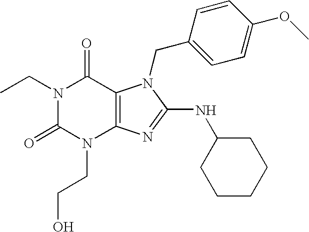 | 442.2454 | 442.2451 | (M + 1) |
| 104 | 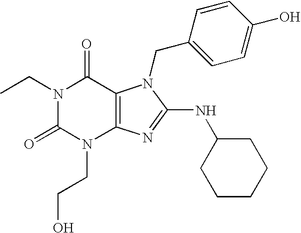 | 428.2298 | 428.2294 | (M + 1) |
| 105 | 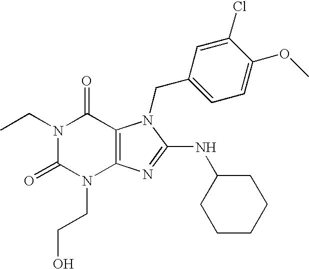 | 476.2065 | 476.2057 | (M + 1) |
| 106 | 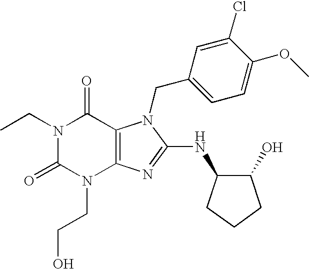 | 478.1857 | 478.1851 | (M + 1) |
| 107 | 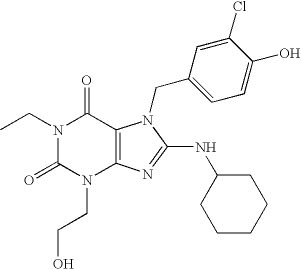 | 462.1908 | 462.191 | (M + 1) |
| 108 | 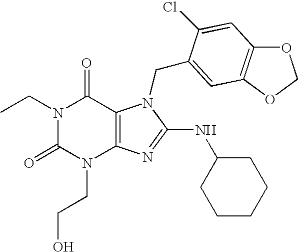 | 490.1857 | 490.1853 | (M + 1) |
| 109 | 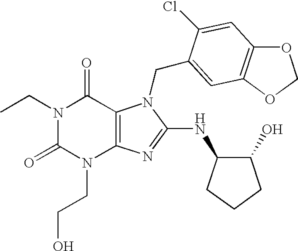 | 492.1650 | 492.1641 | (M + 1) |
| 110 | 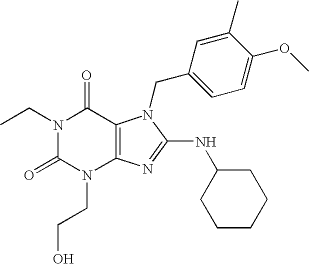 | 455.2533 | 455.2518 | (M+) |
| 111 | 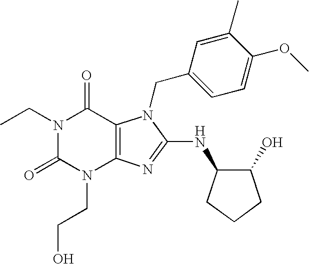 | 458.2403 | 458.2395 | (M + 1) |
| 112 | 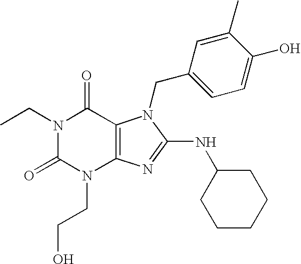 | 442.2454 | 442.2448 | (M + 1) |
| 113 | 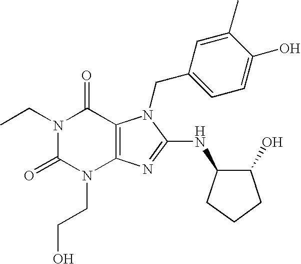 | 444.2247 | 444.2252 | (M + 1) |
| 114 | 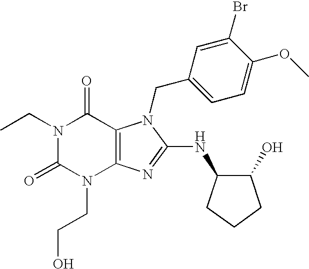 | 522.1352 | 522.1346 | (M + 1) |
| 115 | 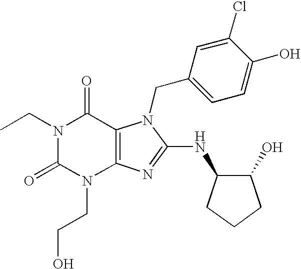 | 464.1701 | 464.1696 | (M + 1) |
| 116 | 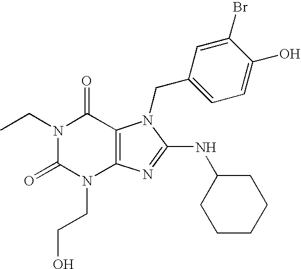 | 506.1403 | 506.141 | (M + 1) |
| 117 | 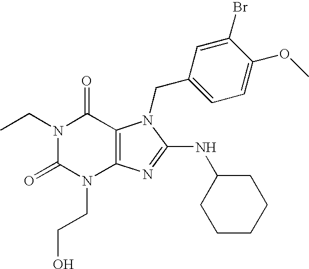 | 520.1559 | 520.1568 | (M + 1) |
| 118 | 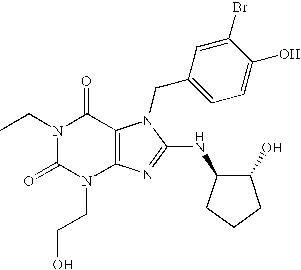 | 508.1196 | 508.119 | (M + 1) |
| 119 | 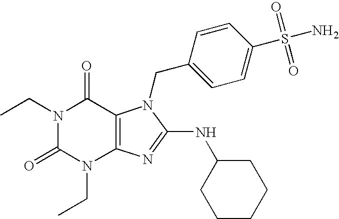 | 475.2128 | 475.2134 | (M + 1) |
| 120 | 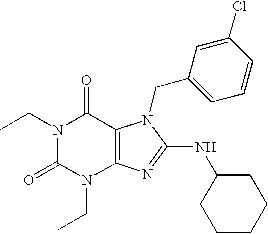 | 429.1932 | 429.1931 | (M+) |
| 121 | 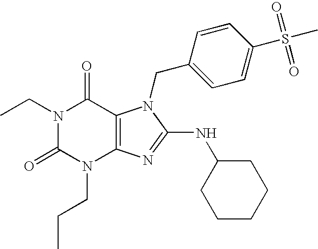 | 488.2332 | 488.2333 | (M + 1) |
| 122 | 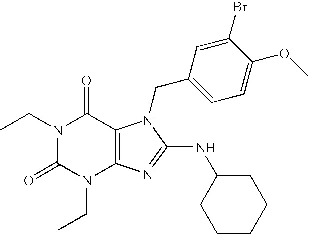 | 504.1610 | 504.1605 | (M + 1) |
| 123 | 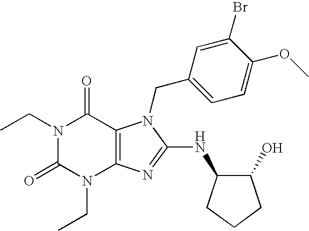 | 506.1403 | 506.1395 | (M + 1) |
| 124 | 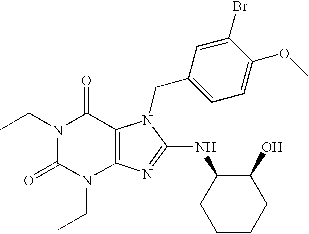 | 522.1542 | 522.1542 | (M + 1) |
| 125 | 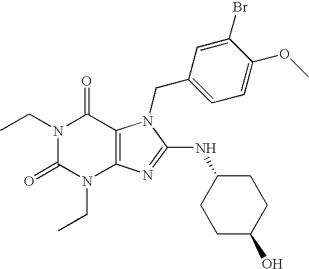 | 520.1559 | 520.1552 | (M + 1) |
| 126 | 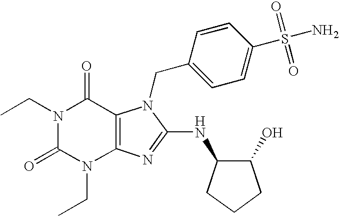 | 477.1920 | 477.1919 | (M + 1) |
| 127 | 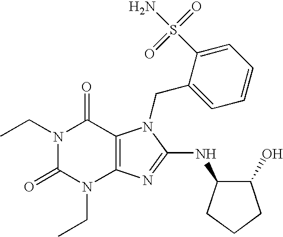 | 477.1920 | 477.1914 | (M + 1) |
| 128 | 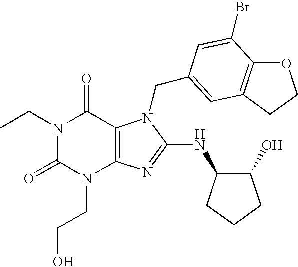 | 536.1335 | 536.1335 | (M + 1) |
| 129 | 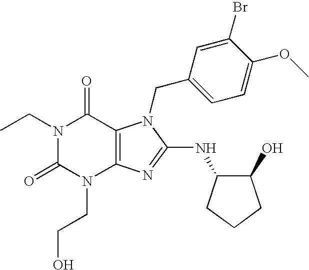 | 522.1352 | 522.136 | (M + 1) |
| 130 | no structure | n/a | n/a | n/a |
| 131 | 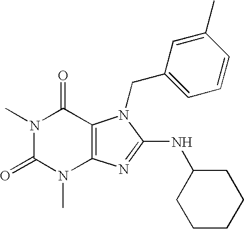 | 382.2243 | 382.2242 | (M + 1) |
| 132 | 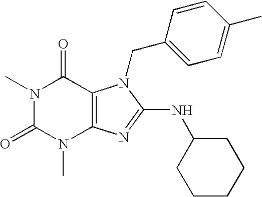 | 382.2243 | 382.2238 | (M + 1) |
| 133 | 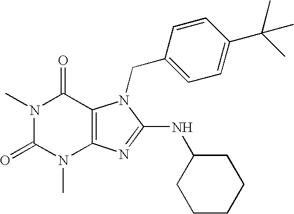 | 424.2713 | 424.2717 | (M + 1) |
| 134 | 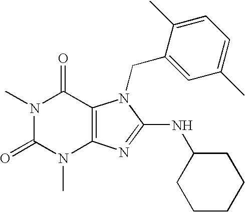 | 396.2400 | 396.2396 | (M + 1) |
| 135 | 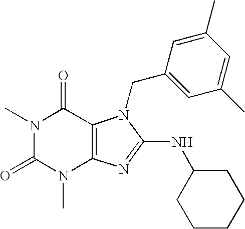 | 396.2400 | 396.2393 | (M + 1) |
| 136 | 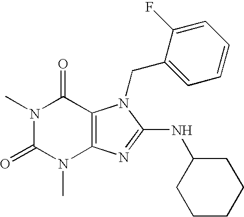 | 386.1992 | 386.1988 | (M + 1) |
| 137 | 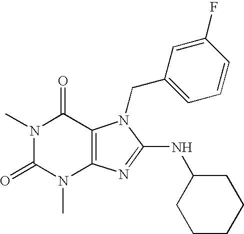 | 386.1992 | 386.1988 | (M + 1) |
| 138 | 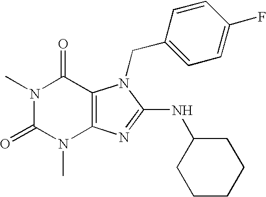 | 386.1992 | 386.1985 | (M + 1) |
| 139 | 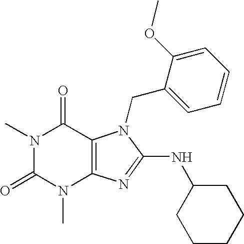 | 398.2192 | 398.2196 | (M + 1) |
| 140 | 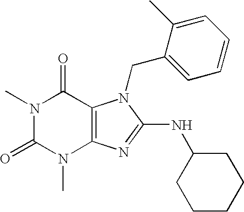 | 382.2243 | 382.2238 | (M + 1) |
| 141 | 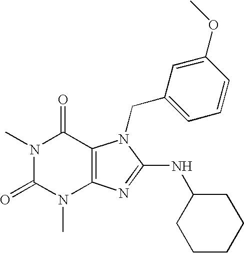 | 398.2192 | 398.2192 | (M + 1) |
| 142 | 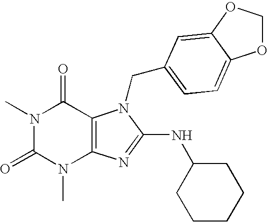 | 412.1985 | 412.1982 | (M + 1) |
| 143 | 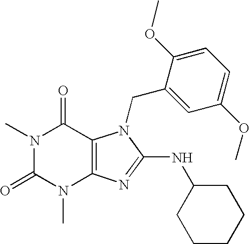 | 428.2298 | 428.2294 | (M + 1) |
| 144 | 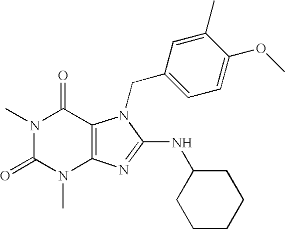 | 412.2349 | 412.2346 | (M + 1) |
| 145 | 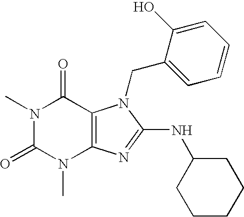 | 384.2036 | 384.2041 | (M + 1) |
| 146 | 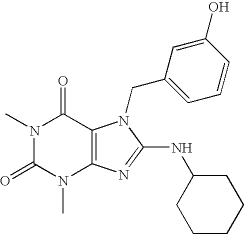 | 384.2036 | 384.2033 | (M + 1) |
| 147 | 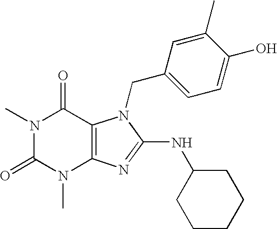 | 398.2192 | 398.2184 | (M + 1) |
| 148 | 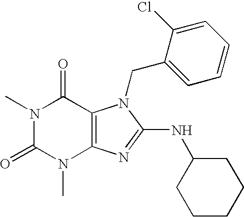 | 402.1697 | 402.1691 | (M + 1) |
| 149 | 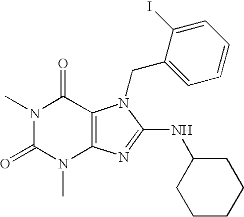 | 493.0975 | 493.098 | (M+) |
| 150 | 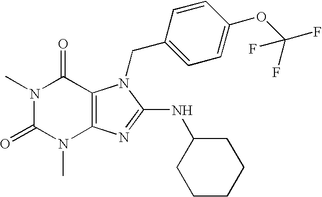 | 451.1831 | 451.1819 | (M+) |
| 151 | 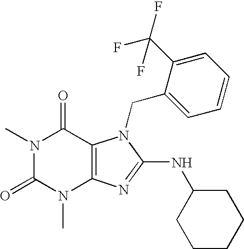 | 435.1882 | 435.1879 | (M+) |
| 152 | 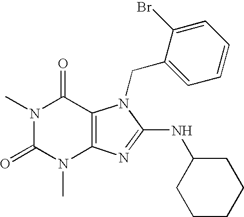 | 446.1192 | 446.1187 | (M + 1) |
| 153 | 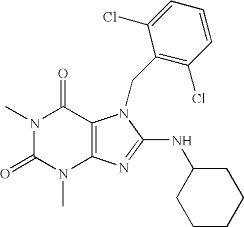 | 435.1229 | 435.1219 | (M+) |
| 154 | 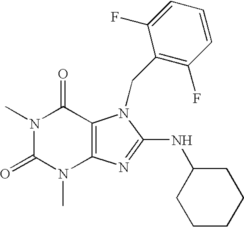 | 404.1898 | 404.1895 | (M + 1) |
| 155 | 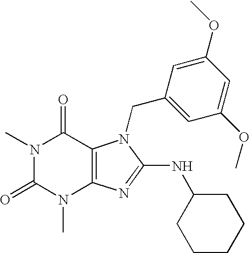 | 428.2298 | 428.2292 | (M + 1) |
| 156 | 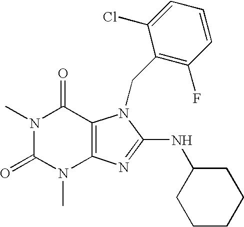 | 420.1603 | 420.1603 | (M + 1) |
| 157 | 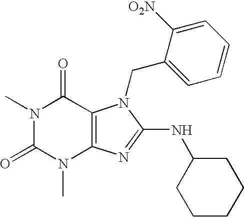 | 413.1937 | 413.1932 | (M + 1) |
| 158 | 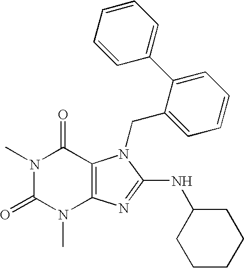 | 444.2400 | 444.2394 | (M + 1) |
| 159 | 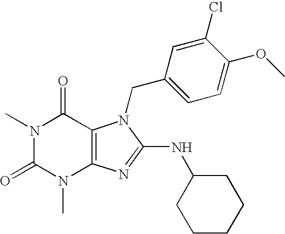 | 431.1724 | 431.173 | (M+) |
| 160 | 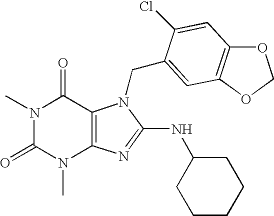 | 446.1595 | 446.1588 | (M + 1) |
| 161 | 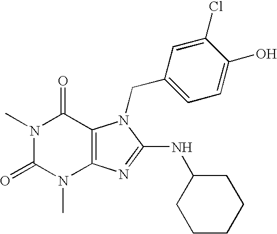 | 418.1646 | 418.164 | (M + 1) |
| 162 | 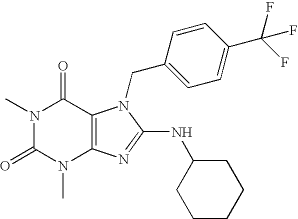 | 436.1960 | 436.1962 | (M + 1) |
| 163 | 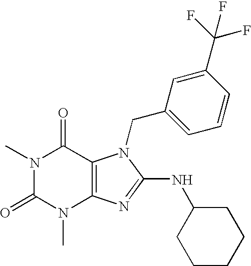 | 436.1960 | 436.1957 | (M + 1) |
| 164 | 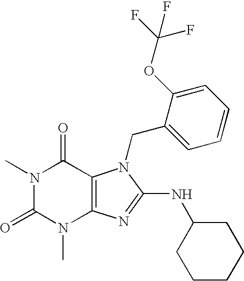 | 452.1909 | 452.1919 | (M + 1) |
| 165 | 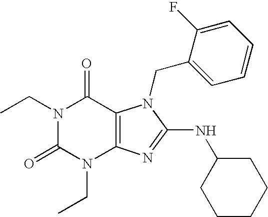 | 414.2305 | 414.2303 | (M + 1) |
| 166 | 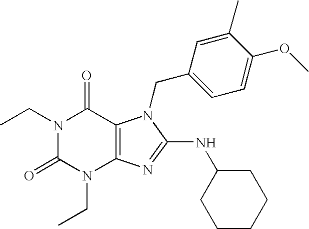 | 440.2662 | 440.2657 | (M + 1) |
| 167 | 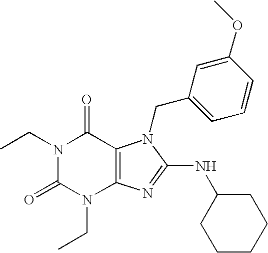 | 426.2505 | 426.2509 | (M + 1) |
| 168 | 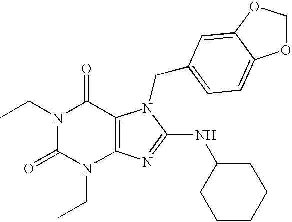 | 440.2298 | 440.2295 | (M + 1) |
| 169 | 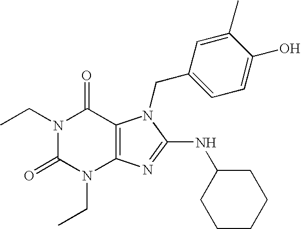 | 426.2505 | 426.2498 | (M + 1) |
| 170 | 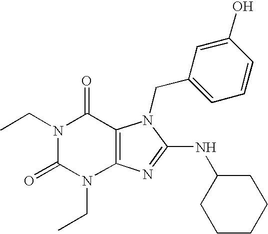 | 412.2349 | 412.2345 | (M + 1) |
| 171 | 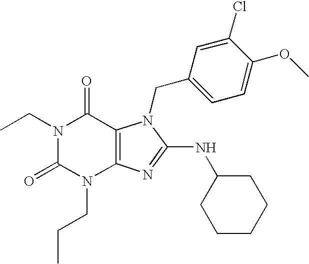 | 474.2272 | 474.2277 | (M + 1) |
| 172 | 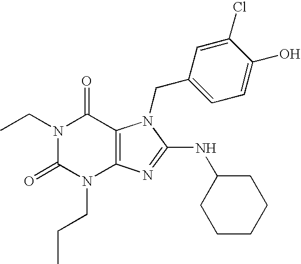 | 459.2037 | 459.2055 | (M+) |
| 173 | 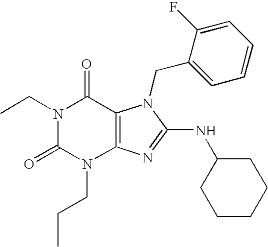 | 428.2462 | 428.2457 | (M + 1) |
| 174 | 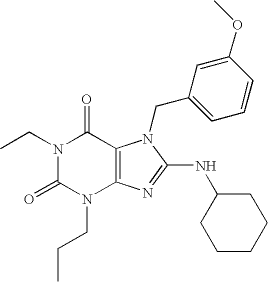 | 440.2662 | 440.2657 | (M + 1) |
| 175 | 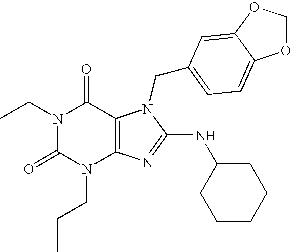 | 454.2454 | 454.2449 | (M + 1) |
| 176 | 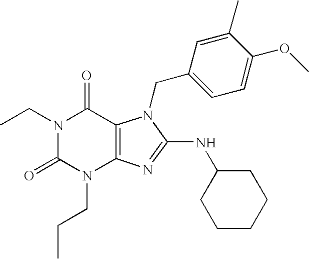 | 454.2818 | 454.2812 | (M + 1) |
| 177 | 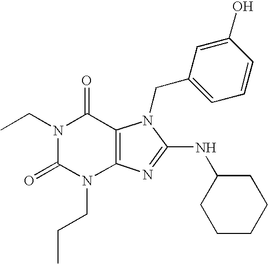 | 426.2505 | 426.2503 | (M + 1) |
| 178 | 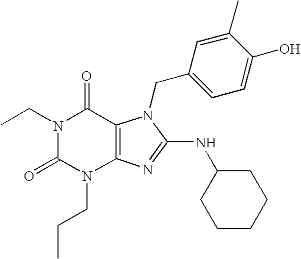 | 440.2662 | 440.2666 | (M + 1) |
| 179 | 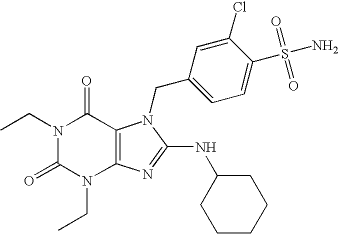 | 509.1738 | 509.1729 | (M + 1) |
| 180 | 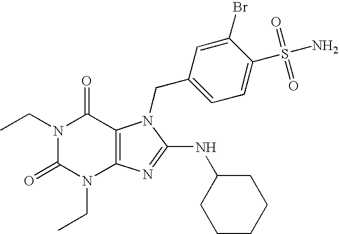 | 555.1233 | 555.123 | (M + 1) |
| 181 | 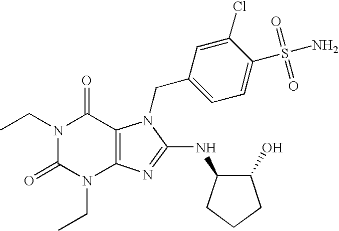 | 511.153 | 511.1524 | (M + 1) |
| 182 | 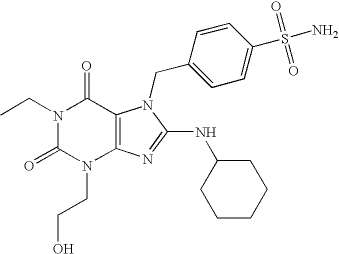 | 491.2077 | 491.2087 | (M + 1) |
| 183 | 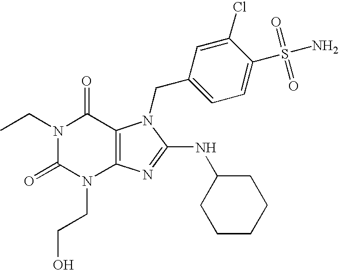 | 525.1687 | 525.1697 | (M + 1) |
| 184 | 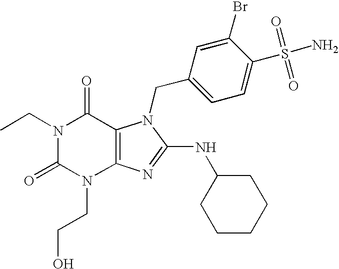 | 571.1164 | 571.1138 | (M + 1) |
| 185 | 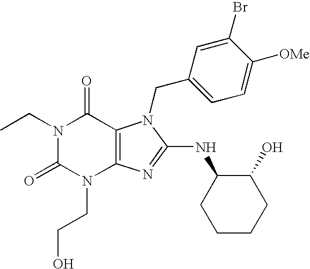 | 538.1492 | 538.1498 | (M + 1) |
| 186 | 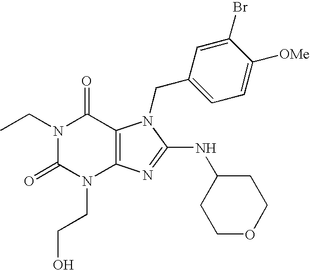 | 524.1335 | 524.1344 | (M + 1) |
| 187 | 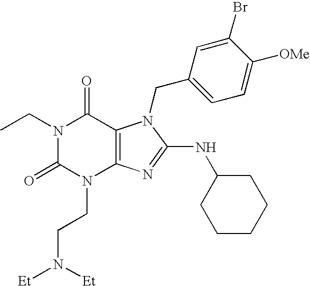 | 575 | 575 | (M) LRMS |
| 188 | 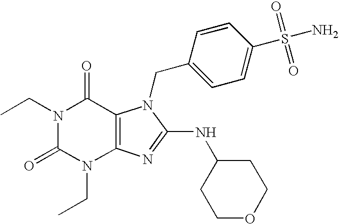 | 477.192 | 477.1919 | (M + 1) |
| 189 | 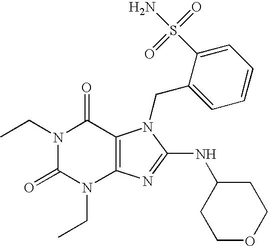 | 477.192 | 477.1919 | (M + 1) |
| 190 | 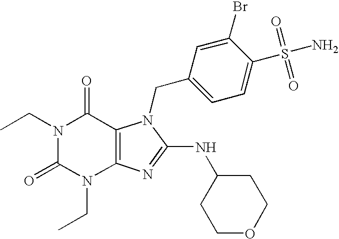 | 557.1007 | 557.0997 | (M + 1) |
| 191 | 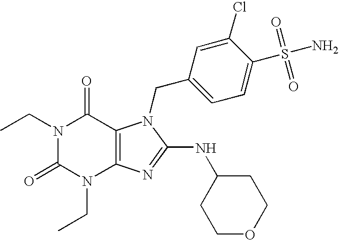 | 511.153 | 511.1519 | (M + 1) |
| 192 | 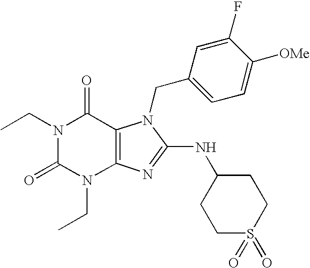 | 494.1637 | 494.1636 | (M + 1) |
| 193 | 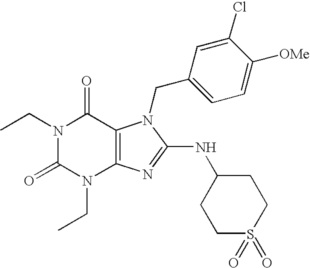 | 510.1578 | 510.1574 | (M + 1) |
| 194 | 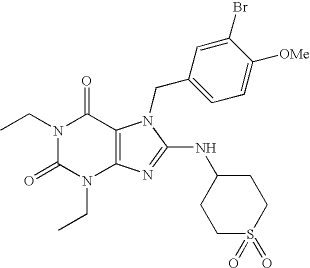 | 554.1073 | 554.1066 | (M + 1) |
| 195 | 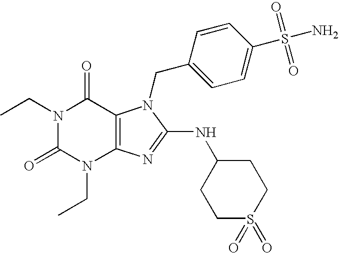 | 525.159 | 525.1582 | (M + 1) |
| 196 | 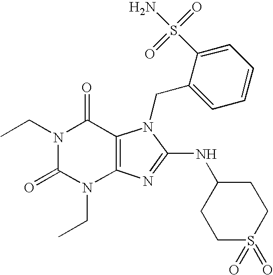 | 525.159 | 525.1597 | (M + 1) |
| 197 | 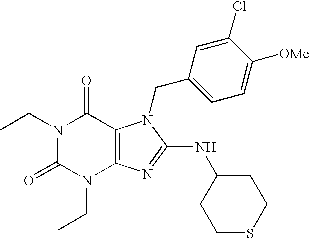 | 478.168 | 478.1683 | (M + 1) |
| 198 | 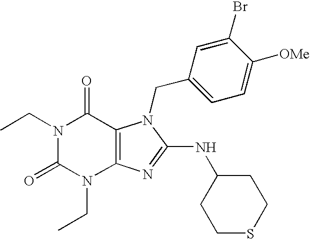 | 522.1174 | 522.1169 | (M + 1) |
| 199 | 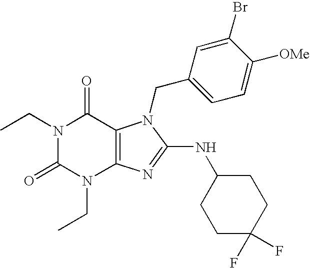 | 542.1405 | 542.143 | (M + 1) |

These compounds are useful for inhibiting PDE V receptors. Their receptor activities and receptor selectivities can be evaluated in a number of ways. In particular, receptor activity can be measured by the PDE V IC₅₀ value, which is the concentration (in nM) of the compound required to provide 50% inhibition of PDE V. The lower the value of IC₅₀, the more active is the compound. Measurements on the compounds in Tables I and II gave the following data (all numbers are modified by the word “about”):
A. all compounds had a PDE V IC₅₀ within the range of from <1 nM to >100 nM;
B. compound nos. 13-18, 25, 30-32, 38, 41-43, 55-58, 69-71, 77, 85, 92, 96, 98, 101, 113, 120, 121, 126, 128, 131, 137, 138, 141, 146-48, 165, 166, 173, 176, 181, 182, 184, 185, 193 and 194 had a PDE V IC₅₀ within the range of from >15 to 100 nM;
C. compound nos. 23, 24, 29, 33, 34, 39, 40, 93, 94, 108, 111, 112, 125, 136, 144, 160 and 161 had a PDE V IC₅₀ within the range of from >10 to 15 nM.
D. compound nos. 21, 22, 28, 36, 37, 59, 66, 68, 78, 79, 89, 95, 99, 110, 115, 132, 159, 171, 172, 175, 180, 183, 190 and 199 had a PDE V IC₅₀ within the range of from >5 to 10 nM; and
E. compound nos. 60-65, 67, 103-07, 114, 116-19, 122-24, 142, 168-70, 177, 178, 179, 186-88, 191, 197 and 198 had a PDE V IC₅₀ within the range of up to 5 nM.

In addition, another type of measurement that can be made is the ratio of PDE VI IC₅₀/PDE V IC₅₀ (identified as “PDE VI/PDE V”), which is an indicator of enzyme selectivity—the higher the ratio, the more selective is the compound to inhibiting PDE V enzyme relative to PDE VI enzyme. Measurements on the compounds (except for compound nos. 189, 192, 195 and 196) in Table II gave the following data (all numbers are modified by the word “about”):
F. compound nos. 1-188, 190, 191, 193, 194 and 197-99 had a PDE VI/PDE V ratio of >0;
G. compound nos. 165 and 193 had a PDE VI/PDE V ratio within the range of from >0 to 10;
H. compound nos. 101, 108, 136, 141, 146, 148, 168, 173 and 194 had a PDE VI/PDE V ratio within the range of from >10 to 25;
I. compound nos. 104, 125, 131-32, 137-38, 142, 144, 170, 175, 177, 185 and 199 had a PDE VI/PDE V ratio within the range of from >25 to 50;
J. compound nos. 103, 110, 111, 117, 159, 166, 182 and 187 had a PDE VI/PDE V ratio within the range of from >50 to 75;
K. compound nos. 105, 106, 147 and 171 had a PDE VI/PDE V ratio within the range of from >75 to 100;
L. compound nos. 112, 113, 123, 124, 126, 169, 172 and 184 had a PDE VI/PDE V ratio within the range of from >100 to 140; and
M. compound nos. 107, 114-16, 118-22, 128, 160-61, 176, 178-81, 183, 186, 188, 190, 191, 197 and 198 had a PDE VI/PDE V ratio of from >140.

Preferred compounds of U.S. Pub. No. 2002/0169174 include those found in classes E and/or M: compound nos. 60-65, 67, 103-07, 114-24, 128, 142, 160-61, 168-70, 176-78, 179, 186, 188, 191, 197 and 198. More preferred are compound nos. 107, 114, 116, 118, 119, 122, 160, 178 and 186 of Table II.

Another preferred compound of the invention would have the following chemical structure:

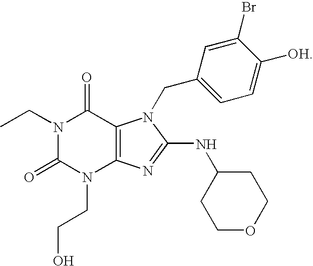

Specific and general procedures for producing three preferred compounds are disclosed in U.S. Ser. No. 08/940,760. Obvious modifications to these procedures may be undertaken by one of ordinary skill in the art. Other compounds of the invention may be produced using similar synthesis schemes.

### Pharmaceutically-Acceptable Dosage Forms

The compounds of the present invention may be administered to humans or other mammals by a variety of routes, including oral dosage forms and injections (intravenous, intramuscular, intraperitoneal, subcutaneous, and the like). Numerous other dosage forms containing the compounds of the present invention can be readily formulated by one skilled in the art, utilizing the suitable pharmaceutical excipients as defined below. For considerations of patient compliance, oral dosage forms are generally most preferred.

The rate of systemic delivery can be satisfactorily controlled by one skilled in the art, by manipulating any one or more of the following:
(a) the active ingredient proper;
(b) the pharmaceutically-acceptable excipient(s), so long as the variants do not interfere in the activity of the particular active ingredient selected;
(c) the type of excipient(s), and the concomitant desirable thickness and permeability (swelling properties) of the excipient(s);
(d) the time-dependent conditions of the excipient(s);
(e) the particle size of the granulated active ingredient; and
(f) the pH-dependent conditions of the excipient(s).

Pharmaceutically-acceptable excipients include flavoring agents, pharmaceutical-grade dyes or pigments, solvents, co-solvents, buffer systems, surfactants, preservatives, sweetener agents, viscosity agents, fillers, lubricants, glidants, disintegrants, binders and resins.

Conventional flavoring agents may be used, such as those described in Remington's Pharmaceutical Sciences, 18^{th} Ed., Mack Publishing Co., pp. 1288-1300 (1990), which is incorporated in its entirety by reference herein. The pharmaceutical compositions of the invention generally contain from about 0 to about 2% of flavoring agents.

Conventional dyes and/or pigments may also be used, such as those described in the Handbook of Pharmaceutical Excipients, by the American Pharmaceutical Association & the Pharmaceutical Society of Great Britain, pp. 81-90 (1986), which is incorporated in its entirety by reference herein. The pharmaceutical compositions of the invention generally contain from about 0 to about 2% of dyes and/or pigments.

The pharmaceutical compositions of the invention generally contain from about 0.1 to about 99.9% of solvent(s). A preferred solvent is water. Preferred co-solvents include ethanol, glycerin, propylene glycol, polyethylene glycol, and the like. The pharmaceutical compositions of the invention may include from about 0 to about 50% of co-solvents.

Preferred buffer systems include acetic, boric, carbonic, phosphoric, succinic, malaic, tartaric, citric, acetic, benzoic, lactic, glyceric, gluconic, glutaric and glutamic acids and their sodium, potassium and ammonium salts. Particularly preferred buffers are phosphoric, tartaric, citric and acetic acids and salts thereof. The pharmaceutical compositions of the invention generally contain from about 0 to about 5% of a buffer.

Preferred surfactants include polyoxyethylene sorbitan fatty acid esters, polyoxyethylene monoalkyl ethers, sucrose monoesters and lanolin esters and ethers, alkyl sulfate salts and sodium, potassium and ammonium salts of fatty acids. The pharmaceutical compositions of the invention generally contain from about 0 to about 2% of surfactants.

Preferred preservatives include phenol, alkyl esters of parahydroxybenzoic acid, o-phenylphenol benzoic acid and salts thereof, boric acid and salts thereof, sorbic acid and salts thereof, chlorobutanol, benzyl alcohol, thimerosal, phenylmercuric acetate and nitrate, nitromersol, benzalkonium chloride, cetylpyridinium chloride, methyl paraben and propyl paraben. Particularly preferred preservatives are the salts of benzoic acid, cetylpyridinium chloride, methyl paraben and propyl paraben. The pharmaceutical compositions of the invention generally include from about 0 to about 2% of preservatives.

Preferred sweeteners include sucrose, glucose, saccharin, sorbitol, mannitol and aspartame. Particularly preferred sweeteners are sucrose and saccharin. Pharmaceutical compositions of the invention generally include from about 0 to about 5% of sweeteners.

Preferred viscosity agents include methylcellulose, sodium carboxymethylcellu lose, hydroxypropyl-methylcellulose, hydroxypropylcellulose, sodium alginate, carbomer, povidone, acacia, guar gum, xanthan gum and tragacanth. Particularly preferred viscosity agents are methylcellulose, carbomer, xanthan gum, guar gum, povidone, sodium carboxymethylcellulose, and magnesium aluminum silicate. Pharmaceutical compositions of the invention generally include from about 0 to about 5% of viscosity agents.

Preferred fillers include lactose, mannitol, sorbitol, tribasic calcium phosphate, diabasic calcium phosphate, compressible sugar, starch, calcium sulfate, dextro and microcrystalline cellulose. Pharmaceutical compositions of the invention generally contain from about 0 to about 75% of fillers.

Preferred lubricants/glidants include magnesium stearate, stearic acid and talc. Pharmaceutical compositions of the invention generally include from about 0 to about 7%, preferably, about 1 to about 5% of lubricants/glidants.

Preferred disintegrants include starch, sodium starch glycolate, crospovidone and croscarmelose sodium and microcrystalline cellulose. Pharmaceutical compositions of the invention generally include from about 0 to about 20%, preferably, about 4 to about 15% of disintegrants.

Preferred binders include acacia, tragacanth, hydroxypropylcellulose, pregelatinized starch, gelatin, povidone, hydroxypropylcellulose, hydroxypropylmethylcellulose, methylcellulose, sugar solutions, such as sucrose and sorbitol, and ethylcellulose. Pharmaceutical compositions of the invention generally include from about 0 to about 12%, preferably, about 1 to about 10% of binders.

Additional agents known to a skilled formulator may be combined with the compounds of the invention to create a single dosage form. Alternatively, additional agents may be separately administered to a mammal as part of a multiple dosage form.

For preparing pharmaceutical compositions containing the subject compounds, inert, pharmaceutically acceptable carriers can be either solid or liquid. Solid form preparations include powders, tablets, dispersible granules, capsules, cachets and suppositories. The powders and tablets may be comprised of from about 5 to about 95 weight percent of active ingredient. Suitable solid carriers are known in the art, for example, magnesium carbonate, magnesium stearate, talc, sugar and lactose. Tablets, powders, cachets and capsules can be used as solid dosage forms suitable for oral administration. Examples of pharmaceutically-acceptable carriers and methods of manufacture for various compositions may be found in Remington's Pharmaceutical Sciences, 18^{th} Ed., Mack Publishing Co. (1990), which is incorporated in its entirety by reference herein.

In one solid dosage form embodiment, the PDE V inhibitor drug product is in the form of a film-coated, immediate release tablet, whose core contains mannitol as a diluent, microcrystalline cellulose as a binder, croscarmelose sodium as a disintegrant, and magnesium stearate as a lubricant. This core is coated using an aqueous suspension of a film-coating agent (Opadry® II White Y-30-18037), which is comprised of lactose monohydrate, hypromellose, titanium dioxide, and triacetin.

Liquid form preparations include solutions, suspensions and emulsions. Common liquid form preparations include water and water-propylene glycol solutions for parenteral injection or addition of sweeteners and opacifiers for oral solutions, suspensions and emulsions. Liquid form preparations may also include solutions for intranasal administration.

Aerosol preparations suitable for inhalation include solutions and solids in powder form, which may be combined with a pharmaceutically acceptable carrier, such as an inert compressed gas (e.g., nitrogen).

Also included are solid form preparations that may be converted, shortly before use, to liquid form preparations for either oral or parenteral administration. Such liquid forms include solutions, suspensions and emulsions.

The compounds of the invention may also be delivered transdermally. The transdermal compositions can take the form of creams, lotions, aerosols and emulsions and may be included in a transdermal patch of a matrix or reservoir type as is conventional in the art for this purpose.

The preferred mode of administering the compounds of the invention is oral. Preferably, the pharmaceutical preparation is in a unit dosage form. In such a form, the preparation is subdivided into suitable sized unit doses containing appropriate quantities of the active component, for example, an effective amount to achieve the desired purpose.

The quantity of active ingredient (compound) in a unit dose of preparation may be varied or adjusted from about 0.01 to about 4,000 mg, preferably, from about 0.02 to about 1,000 mg, more preferably, from about 0.3 to about 500 mg, and most preferably, from about 0.04 to about 250 mg, according to the particular application. A typical recommended daily dosage regimen for oral administration can range from about 0.02 to about 2,000 mg/day, in two to four divided doses. For convenience, the total daily dosage may be divided and administered in portions during the day as required. Typically, pharmaceutical compositions of the invention will be administered from about 1 to about 5 times per day, or alternatively, as a continuous infusion. Such administration can be used as a chronic or acute therapy. The amount of active ingredient that may be combined with carrier materials to produce a single dosage form will vary depending upon the host treated and the particular mode of administration. A typical preparation will contain from about 5 to about 95% of active compound (w/w). Preferably, such preparations will contain from about 20 to about 80 wt. % of active compound.

A preferred daily dosage regimen for oral administration is about 5 to about 75 mg/day, in a single dose, or in two to four divided doses. Dosages of about 50 to about 75 mg/day may be more preferred.

The pharmaceutically-acceptable carriers employed in conjunction with the compounds of the present invention are used at a concentration sufficient to provide a practical size to dosage relationship. The pharmaceutically-acceptable carriers, in total, may comprise from about 0.1 to about 99.9% by weight of the pharmaceutical compositions of the invention, preferably, from about 20 to about 80% by weight.

Upon improvement of a patient's condition, a maintenance dose of a compound, composition or combination of the invention may be administered, if necessary. Subsequently, the dosage or frequency of administration, or both, may be reduced, as a function of the symptoms, to a level at which the improved condition is retained. When the symptoms have been alleviated to the desired level, treatment should cease. Patients may, however, require intermittent treatment on a long-term basis upon any recurrence of disease symptoms.

Specific dosage and treatment regimens for any particular patient may be varied and will depend upon a variety of factors, including the activity of the specific compound employed, the age, body weight, general health status, sex and diet of the patient, the time of administration, the rate of excretion, the specific drug combination, the severity and course of the symptoms being treated, the patient's disposition to the condition being treated and the judgment of the treating physician. Determination of the proper dosage regimen for a particular situation is within the skill of the art. The amount and frequency of the administration of compounds of the invention or their pharmaceutically acceptable salts may be regulated according to the judgment of the attending clinician, based on the factors recited above. As a skilled artisan will appreciate, lower or higher doses than those recited above may be required.

For example, it is often the case that a proper dosage level is based on the weight of the patient. For instance, dosage levels of between about 0.01 and about 100 mg/kg of body weight per day, preferably, between about 0.5 and about 75 mg/kg of body weight per day, and more preferably, between about 1 and about 50 mg/kg of body weight per day, of the subject compounds, compositions and salts thereof described herein, are therapeutically useful for the treatment of a variety of biological disorders, particularly, male and female sexual dysfunction. Between two patients of differing weights, a higher dosage will be used for the heavier patient, all other things being equal.

The subject compounds can exist in unsolvated as well as solvated forms, including hydrated forms. In general, the solvated forms, with pharmaceutically-acceptable solvents, such as water, ethanol and the like, are equivalent to the unsolvated forms for purposes of this invention.

The subject compounds may form pharmaceutically-acceptable salts with organic and inorganic acids. Examples of suitable acids for salt formation are hydrochloric, sulfuric, phosphoric, acetic, citric, malonic, salicylic, malic, fumaric, succinic, ascorbic, maleic, methanesulfonic and other mineral and carboxylic acids well known to those skilled in the art. The salts are prepared by contacting the free base forms with a sufficient amount of the desired acid to produce a salt in a conventional manner. The free base forms may be regenerated by treating the salt with a suitable dilute aqueous base solution, such as dilute aqueous sodium hydroxide, potassium carbonate, ammonia or sodium bicarbonate. The free base forms may differ somewhat from their respective salt forms in certain physical properties, such as solubility in polar solvents, but the salts are otherwise equivalent to their respective free base forms for purposes of the invention.

The PDE V inhibitor may be employed alone or in combination with other classes of therapeutic agents, particularly, prostanoids, α-adrenergic receptor, dopamine receptor agonists, melanocortin receptor agonists, endothelin receptor antagonists including ET_{A} receptor antagonists, endothelin converting enzyme inhibitors, angiotensin II receptor antagonists, angiotensin converting enzyme inhibitors, neutral metalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5.

Non-limiting examples of specific therapeutic agents that may be used in combination with compounds of the invention include the following: prostanoids, such as prostaglandin E₁; α-adrenergic agonists, such as phentolamine mesylate; dopamine receptor agonists, such as apomorphine; ET_{A} receptor antagonists, such as bosentan, atrasentan, ambrisentan, darusentan, sitaxsentan, ABT-627, TBC-3711, CI-1034, SPP-301, SB-234551, ZD-4054, BQ-123 and BE-18257B; thromboxane A2 biosynthesis inhibitors such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Combinations with ET_{A} receptor antagonists are preferred, based on the dual mechanism of action that would be brought to patients. Among the ET_{A} receptor antagonists, sitaxsentan is particularly selective over ET_{B}, and demonstrates pharmacokinetics best suited to once a day dosing. For these reasons, combinations with sitaxsentan are preferred.

When the invention comprises a combination of a PDE V inhibitor and one or more other therapeutic agents, the two or more active components may be co-administered simultaneously or sequentially, or in a single pharmaceutical composition comprising a PDE V inhibitor compound and the other therapeutic agent(s) in a pharmaceutically acceptable carrier. The components of the combination can be administered individually or together in any conventional dosage form such as capsule, tablet, powder, cachet, suspension, solution, suppository, nasal spray, etc. The dosage of the other therapeutic active agent(s) can be determined from published material, and may range from 1 to about 1000 mg per dose.

In addition to congestive heart failure, other physiological disorders, symptoms and diseases can also be treated by cGMP-PDE V inhibition. More specifically, PDE V inhibitors may be used to treat atherosclerosis, acute coronary syndrome, arrhythmia, heart disease, myocardial infarction, thrombotic or thromboembolytic stroke, a deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, or renal vascular homeostasis. PDE V inhibitor compounds can also be used in combinations with other therapeutic agents as described above to treat these physiological disorders.

Another aspect of this invention is to provide a kit comprising separate containers in a single package, wherein the subject pharmaceutical compounds, compositions and/or salts thereof are used in combination with pharmaceutically-acceptable carriers to treat disorders, symptoms and diseases where cGMP-PDE V inhibition plays a role.

The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

What is claimed is:

1. A method of treating congestive heart failure comprising administering to a patient in need of such treatment an effective amount of a PDE V inhibitor compound, wherein said compound is a compound of Formula (I), an enantiomer, stereoisomer, rotomer, tautomer or a pharmaceutically acceptable salt thereof: 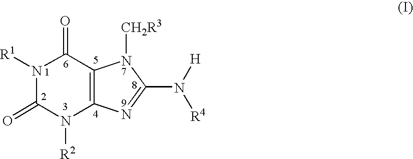
wherein:
(a) R¹ and R² are, independently of one another, each a C₁₋₁₅ alkyl group, branched or straight chain, unsubstituted or substituted with one or more substituents, a C₂₋₁₅ alkenyl group, branched or straight chain, unsubstituted or substituted with one or more substituents, a C₂₋₁₅ alkynyl group, branched or straight chain, unsubstituted or substituted with one or more substituents, or one of R¹ and R² is a hydrogen atom and the other one of R¹ and R² is defined the same as above;
(b) R³ is an aryl group, unsubstituted or substituted with one or more substituents, a heteroaryl group, unsubstituted or substituted with one or more substituents, or a heterocyclic group having 1 to 3 heteroatoms fused to a 5- or 6-membered aryl ring, unsubstituted or substituted with one or more substituents, with the proviso that R³ is not an aryl group substituted at its para position with a —Y-aryl group, where, Y is a carbon-carbon single bond, —C(O)—, —O—, —S—, —N(R²¹)—, —C(O)N(R²²)—, —N(R²²)C(O)—, —OCH₂—, —CH₂O—, —SCH₂—, —CH₂S—, —N(H)C(R²³)(R²⁴)—, —N(R²³)S(O₂)—, —S(O₂)N(R²³)—,
(c) —(R²³)(R²⁴)N(H)—, —CH═CH—, —CF═CF—, —CH═CF—, —CF═CH—, —CH₂CH₂—, —CF₂CF₂—, 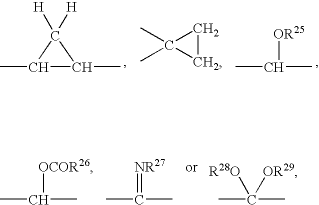
where,
R²¹ is a hydrogen atom or a —CO(C₁₋₄ alkyl), C₁₋₆ alkyl, allyl, C₃₋₆ cycloalkyl, phenyl or benzyl group;
R²² is a hydrogen atom or a C₁₋₆ alkyl group;
R²³ is a hydrogen atom or a C₁₋₅ alkyl, aryl or —CH₂-aryl group;
R²⁴ is a hydrogen atom or a C₁₋₄ alkyl group;
R²⁵ is a hydrogen atom or a C₁₋₈ alkyl, C₁₋₈ perfluoroalkyl, C₃₋₆ cycloalkyl, phenyl or benzyl group;
To R²⁶ is a hydrogen atom or a C₁₋₆ alkyl, C₃₋₆ cycloalkyl, phenyl or benzyl group;
R²⁷ is —NR²³R²⁴, —R²⁴, —NHCONH₂, —NHCSNH₂, 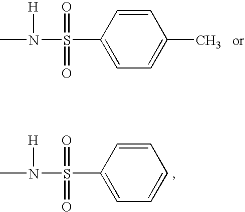
and
R²⁸ and R²⁹ are, independently of one another, each a C₁₋₄ alkyl group or, taken together with each other, a —(CH₂)_{q} group, where q is 2 or 3; and
(d) R⁴ is a C₃₋₁₅ cycloalkyl group, unsubstituted or substituted with one or more substituents, or a C₃₋₁₅ cycloalkenyl group, unsubstituted or substituted with one or more substituents;
wherein, the one or more substituents for all the groups are chemically-compatible and are, independently of one another, each an: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo, nitro, oximino, —COOR⁵⁰—COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ or —N(R⁵²)CON(R⁵⁰R⁵¹) group, where:
R⁵⁰, R⁵¹ and R⁵² are, independently of one another, each a hydrogen atom or a branched or straight-chain, optionally substituted, C₁₋₆ alkyl, C₃₋₆ cycloalkyl, C₄₋₆ heterocycloalkyl, heteroaryl or aryl group, or R⁵⁰ and R⁵¹ are joined together to form a carbocyclic or heterocyclic ring system, or R⁵⁰, R⁵¹ and R⁵² are, independently of one another, each: 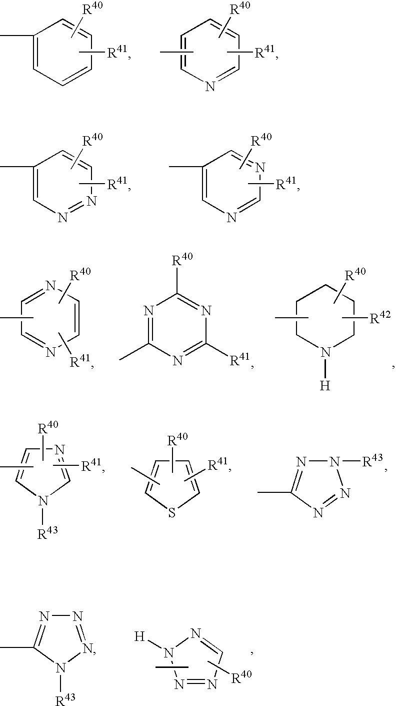
where,
R⁴⁰ and R⁴¹ are, independently of one another, each a hydrogen atom or a branched or straight-chain, optionally substituted, alkyl, cycloalkyl, heterocycloalkyl, halo, aryl, imidazolylalkyl, indolylalkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are defined the same as above;
R⁴² is a hydrogen atom or a branched or straight-chain, optionally substituted, alkyl, alkenyl, arylalkyl or acyl group; and
R⁴³ is a hydrogen atom or a branched or straight-chain, optionally substituted, alkyl or aryl group;
wherein, the optional substituents are defined the same as above for the one or more substituents.

2. The method according to claim 1, wherein R¹ is a methyl or ethyl group, with or without the one or more substituents.

3. The method according to claim 1, wherein R² is a methyl, ethyl, iso-butyl or hydroxyethyl group, with or without the one or more substituents.

4. The method according to claim 1, wherein R³ is a phenyl group, with or without the one or more substituents.

5. The method according to claim 4, wherein the phenyl group for R³ is substituted with at least one halogen atom.

6. The method according to claim 1, wherein R⁴ is a cyclohexyl, hydroxycyclopentyl or tetrahydropyranyl group, with or without the one or more substituents.

7. The method according to claim 1, wherein said compound is selected from the group consisting of those compounds listed in Tables I and II:
TABLE I

| Compound | |
|---|---|
| No. | Structure |
| 10 | 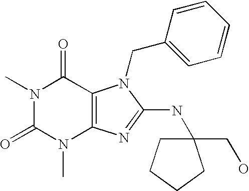 |
| 11 | 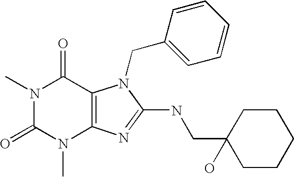 |
| 12 | 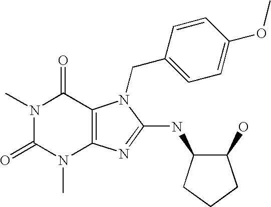 |
| 13 | 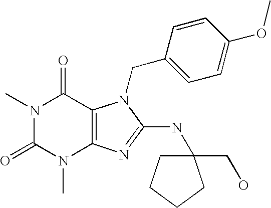 |
| 14 | 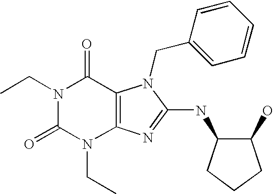 |
| 15 | 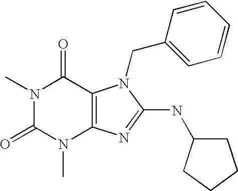 |
| 16 | 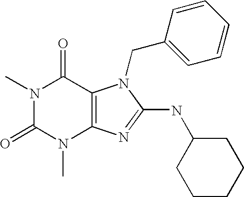 |
| 17 | 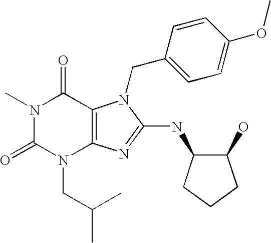 |
| 18 | 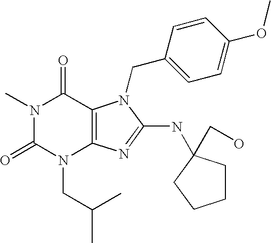 |
| 19 | 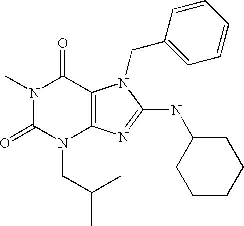 |
| 20 | 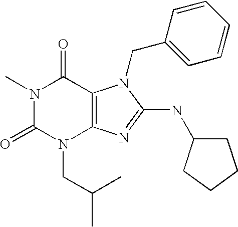 |
| 21 | 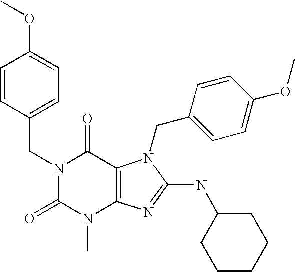 |
| 22 | 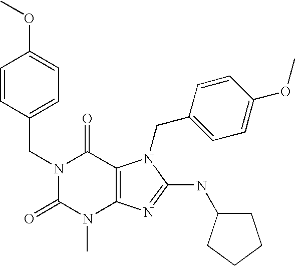 |
| 23 | 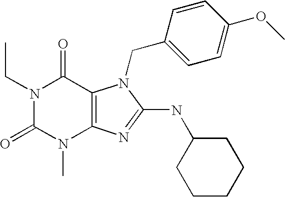 |
| 24 | 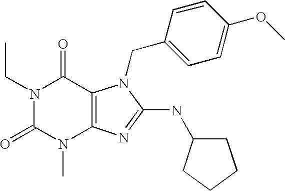 |
| 25 | 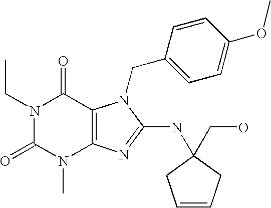 |
| 28 | 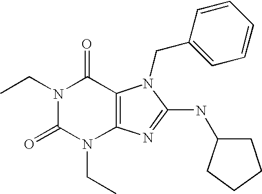 |
| 29 | 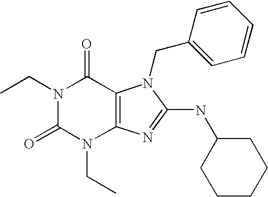 |
| 30 | 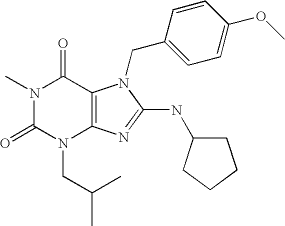 |
| 31 | 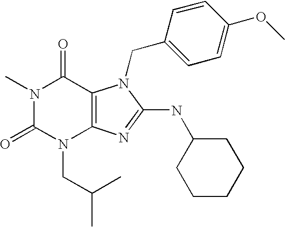 |
| 32 | 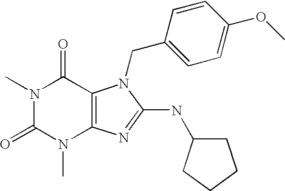 |
| 33 | 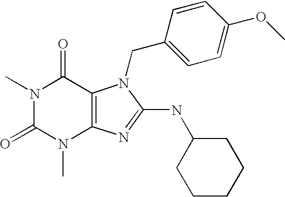 |
| 34 | 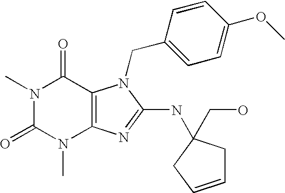 |
| 35 | 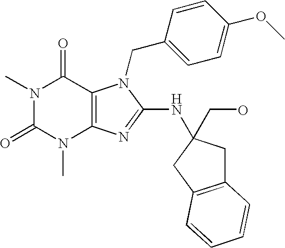 |
| 36 | 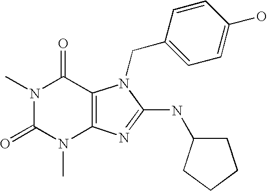 |
| 37 | 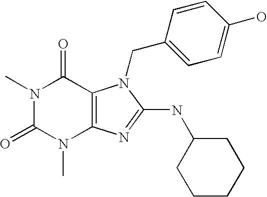 |
| 38 | 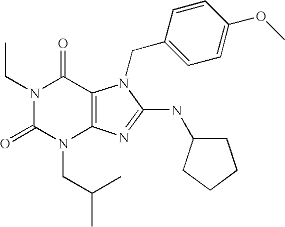 |
| 39 | 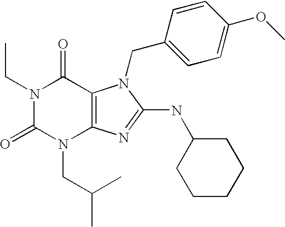 |
| 40 | 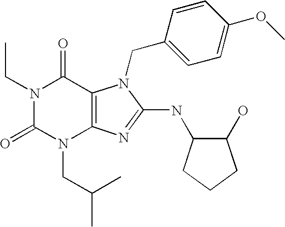 |
| 41 | 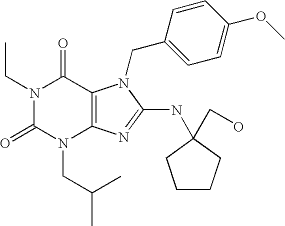 |
| 42 | 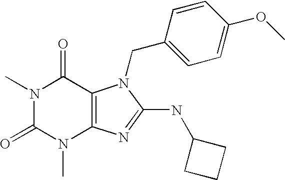 |
| 43 | 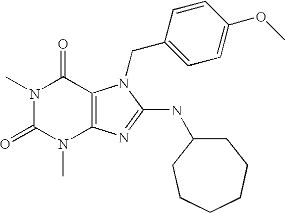 |
| 44 | 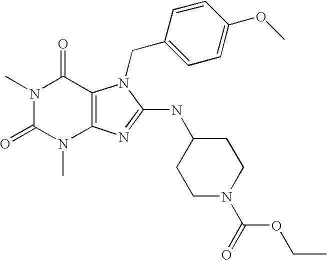 |
| 47 | 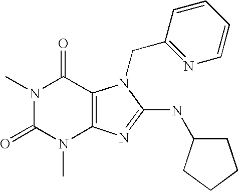 |
| 48 | 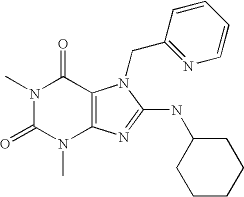 |
| 49 | 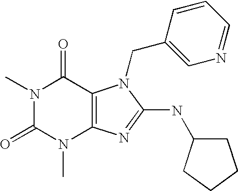 |
| 50 | 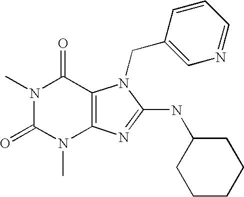 |
| 51 | 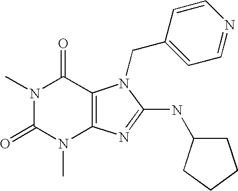 |
| 52 | 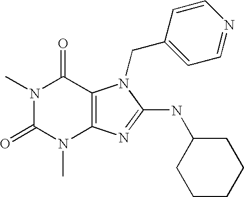 |
| 53 | 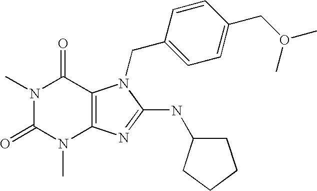 |
| 54 | 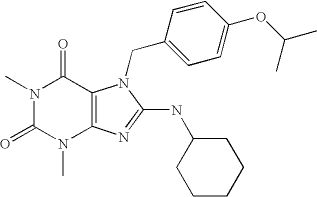 |
| 55 | 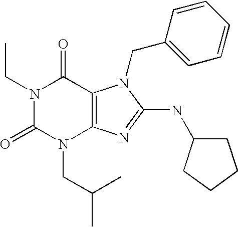 |
| 56 | 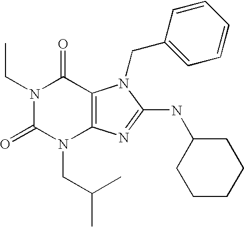 |
| 57 | 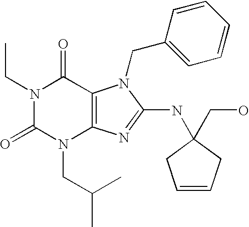 |
| 58 | 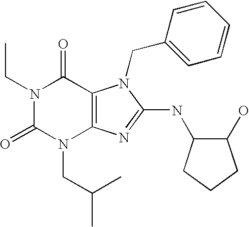 |
| 59 | 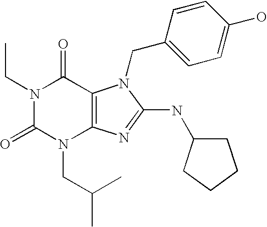 |
| 60 | 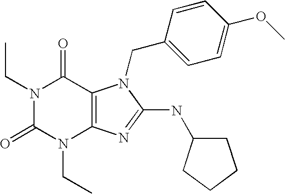 |
| 61 | 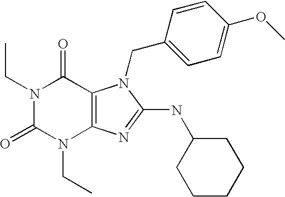 |
| 62 | 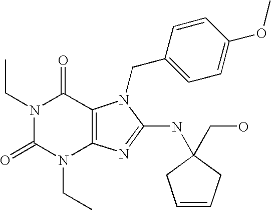 |
| 63 | 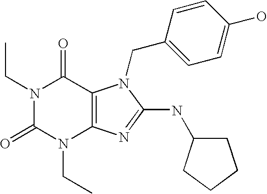 |
| 64 | 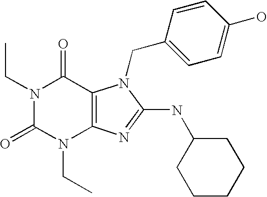 |
| 65 | 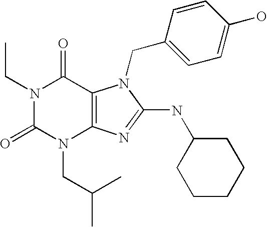 |
| 66 | 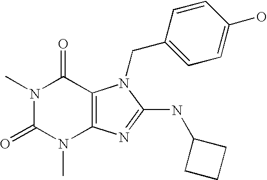 |
| 67 | 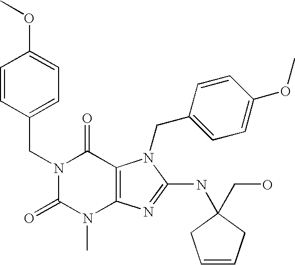 |
| 68 | 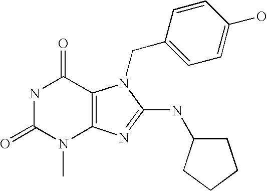 |
| 69 | 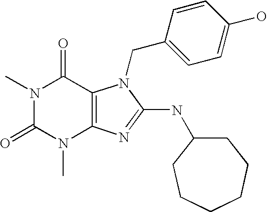 |
| 70 | 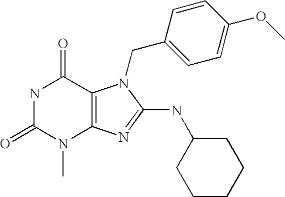 |
| 71 | 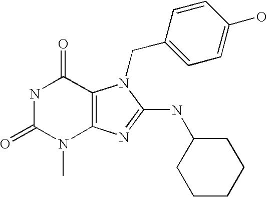 |
| 72 | 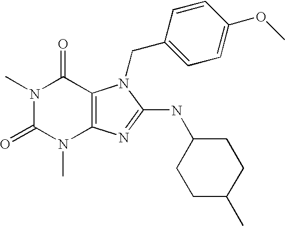 |
| 73 | 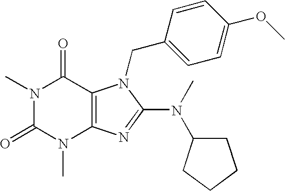 |
| 74 | 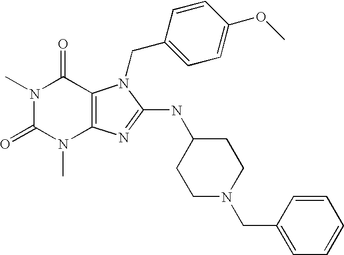 |
| 75 | 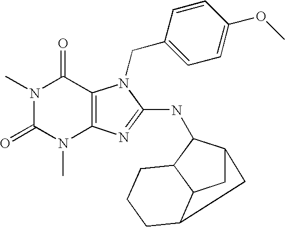 |
| 76 | 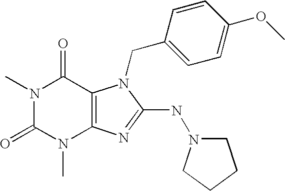 |
| 77 | 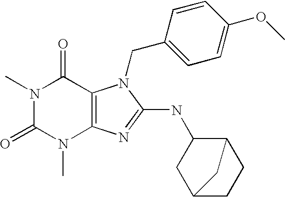 |
| 78 | 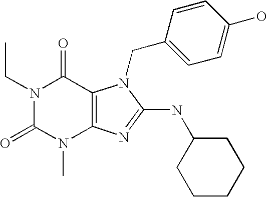 |
| 79 | 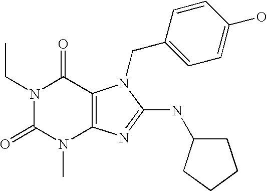 |
| 80 | 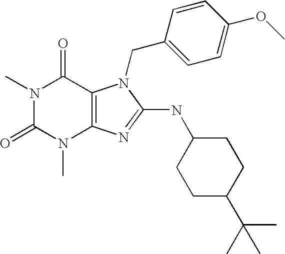 |
| 81 | 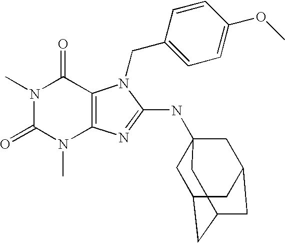 |
| 82 | 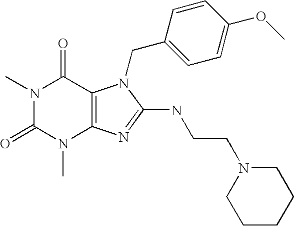 |
| 83 | 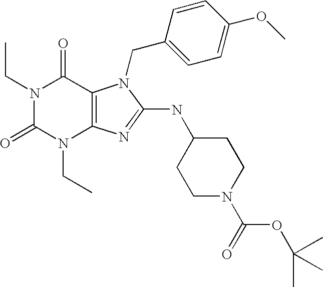 |
| 84 | 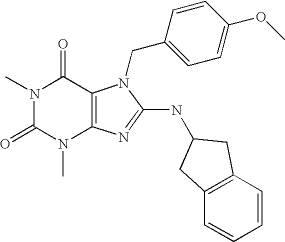 |
| 85 | 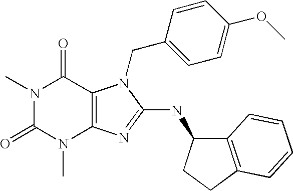 |
| 86 | 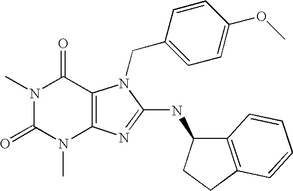 |
| 87 | 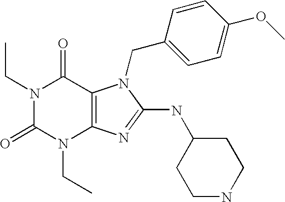 |
| 88 | 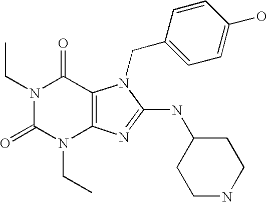 |
| 89 | 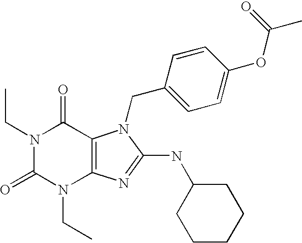 |
| 90 | 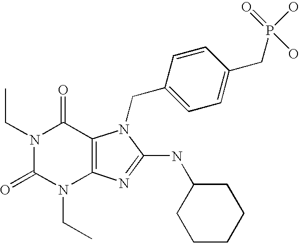 |
| 91 | 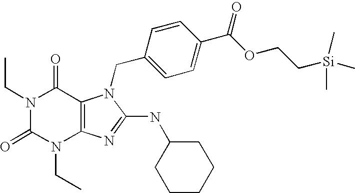 |
| 92 | 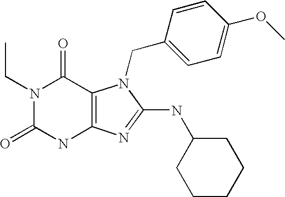 |
| 93 | 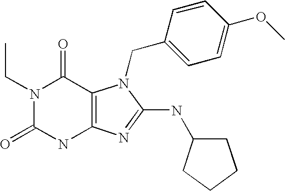 |
| 94 | 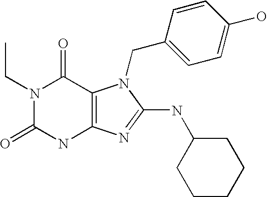 |
| 95 | 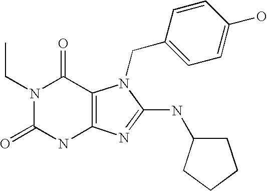 |
| 96 | 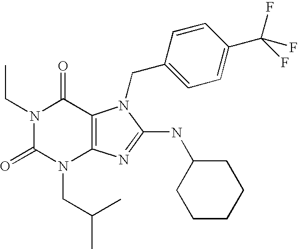 |
| 97 | 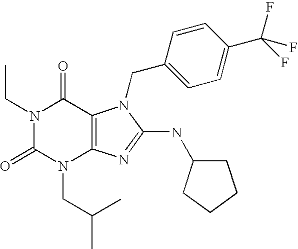 |
| 98 | 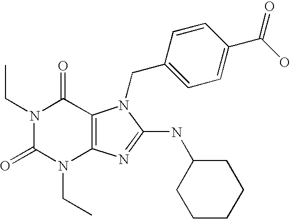 |
| 99 | 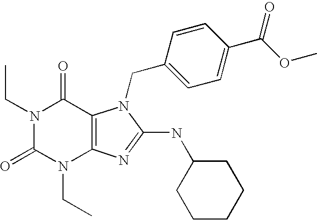 |
| 100 | 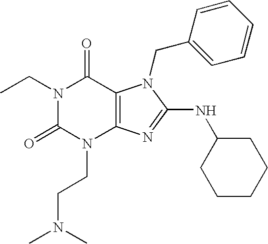 |
| 101 | 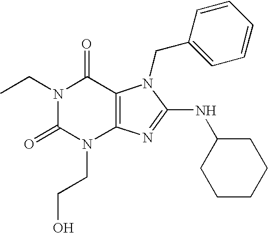 |
| 102 | 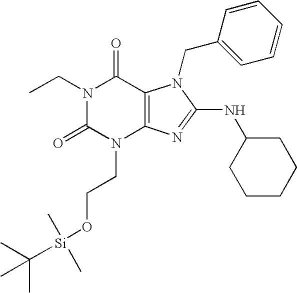 |
| 103 | 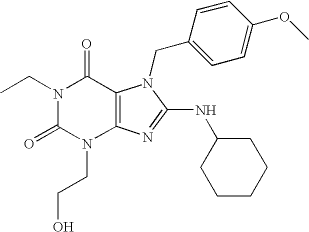 |
| 104 | 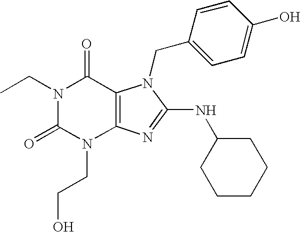 |
| 105 | 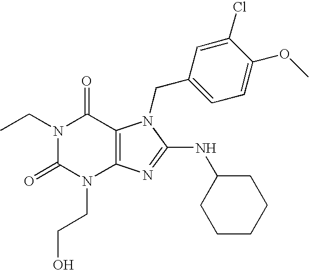 |
| 106 | 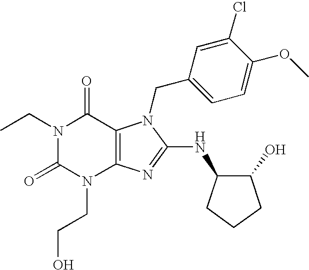 |
| 107 | 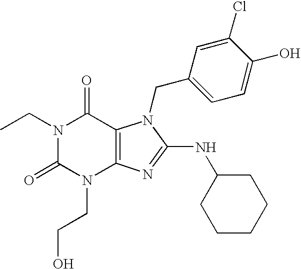 |
| 108 | 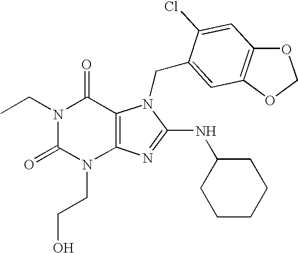 |
| 109 | 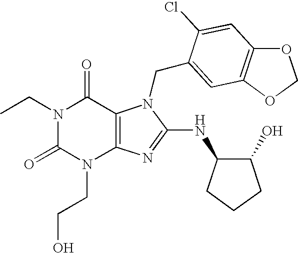 |
| 110 | 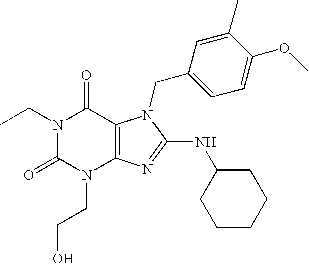 |
| 111 | 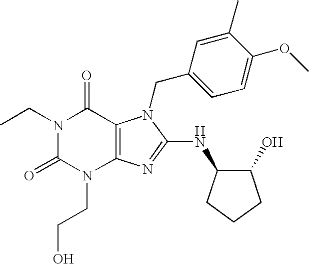 |
| 112 | 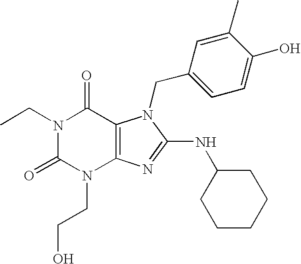 |
| 113 | 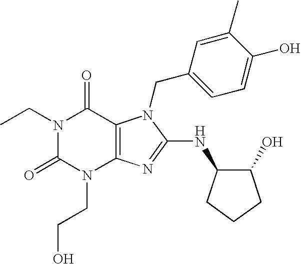 |
| 114 | 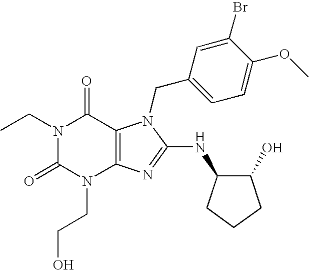 |
| 115 | 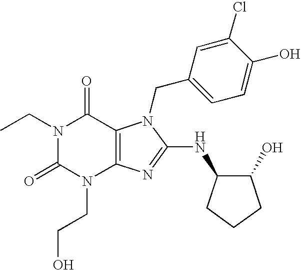 |
| 116 | 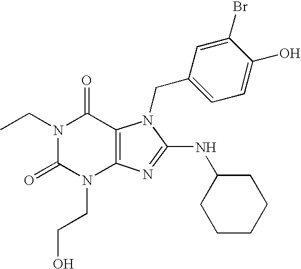 |
| 117 | 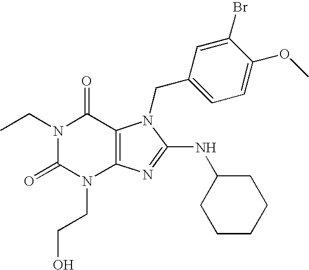 |
| 118 | 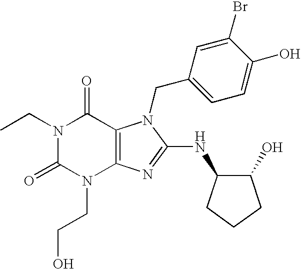 |
| 119 | 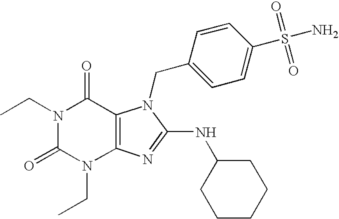 |
| 120 | 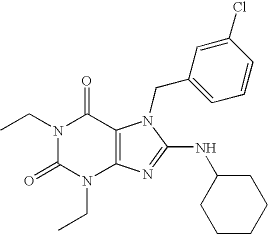 |
| 121 | 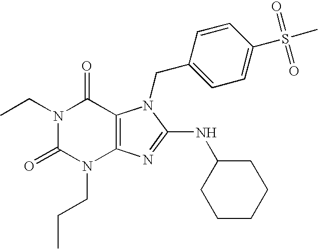 |
| 122 | 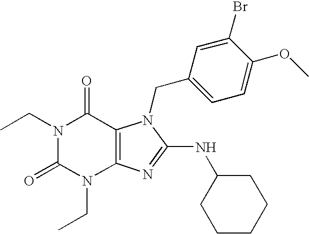 |
| 123 | 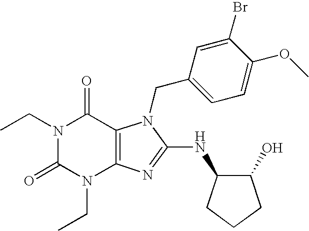 |
| 124 | 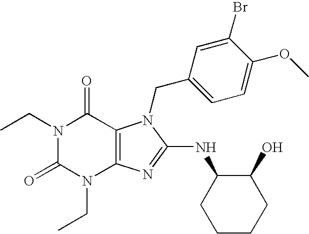 |
| 125 | 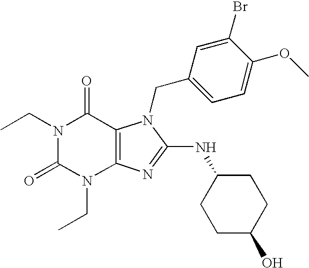 |
| 126 | 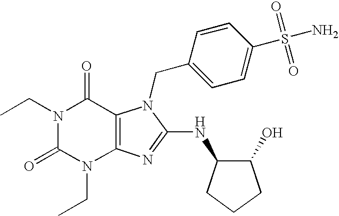 |
| 127 | 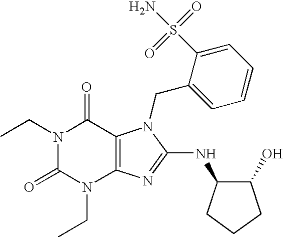 |
| 128 | 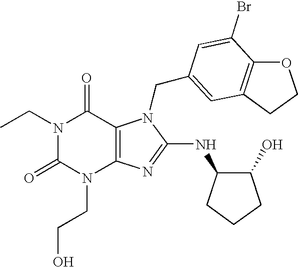 |
| 129 | 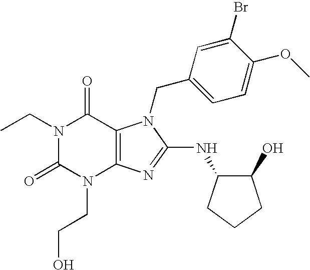 |
| 130 | no structure |
| 131 | 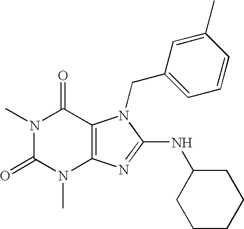 |
| 132 | 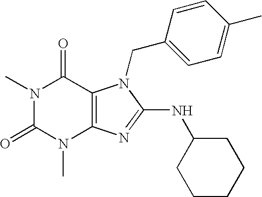 |
| 133 | 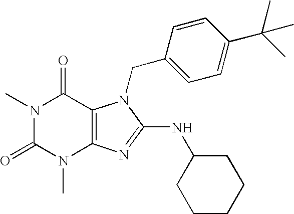 |
| 134 | 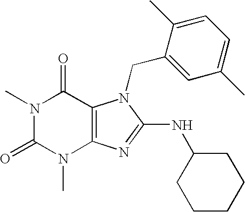 |
| 135 | 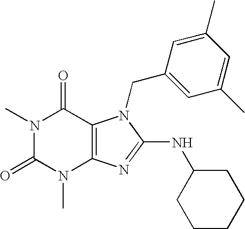 |
| 136 | 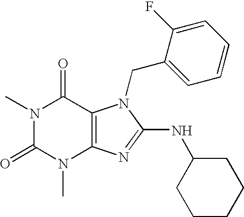 |
| 137 | 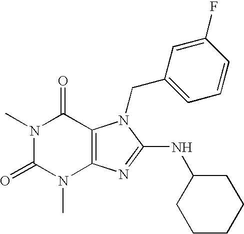 |
| 138 | 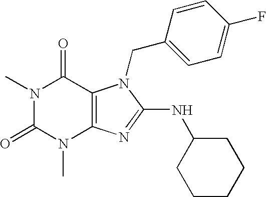 |
| 139 | 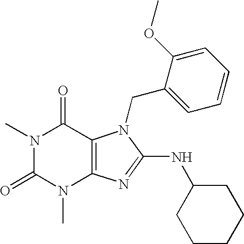 |
| 140 | 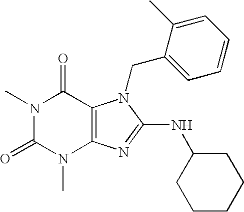 |
| 141 | 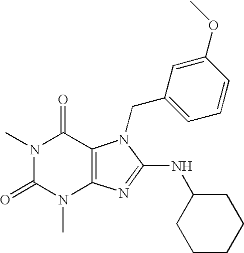 |
| 142 | 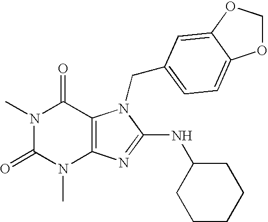 |
| 143 | 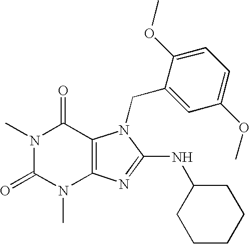 |
| 144 | 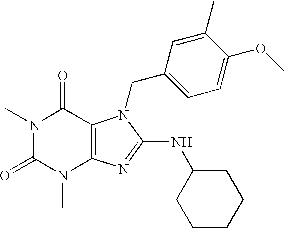 |
| 145 | 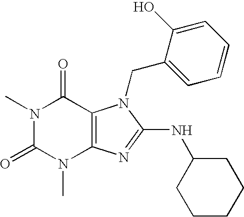 |
| 146 | 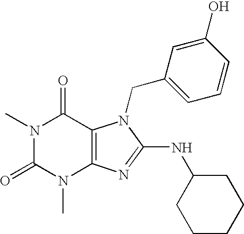 |
| 147 | 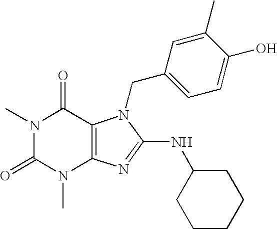 |
| 148 | 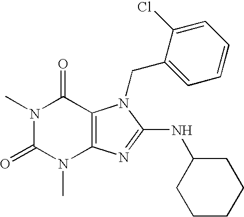 |
| 149 | 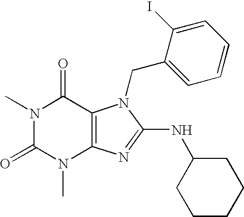 |
| 150 | 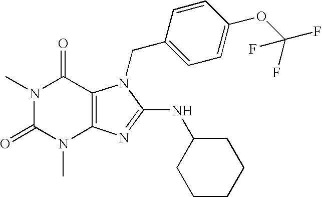 |
| 151 | 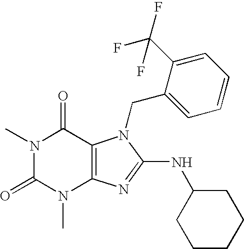 |
| 152 | 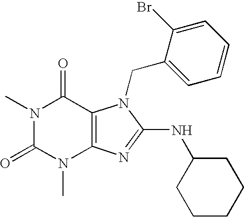 |
| 153 | 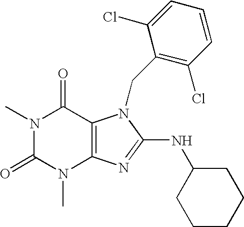 |
| 154 | 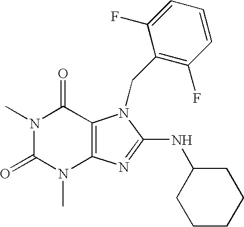 |
| 155 | 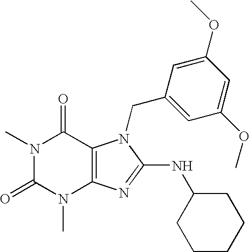 |
| 156 | 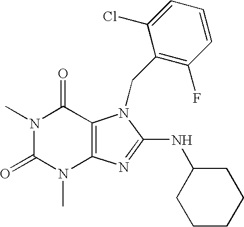 |
| 157 | 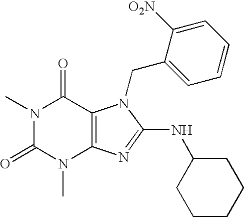 |
| 158 | 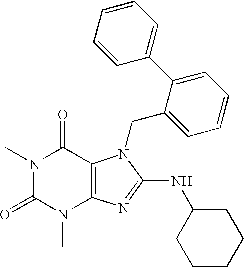 |
| 159 | 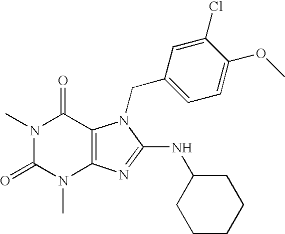 |
| 160 | 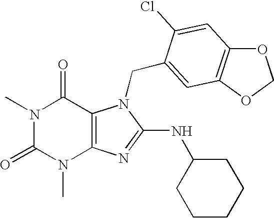 |
| 161 | 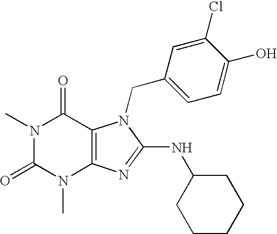 |
| 162 | 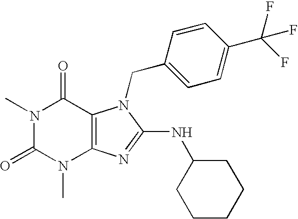 |
| 163 | 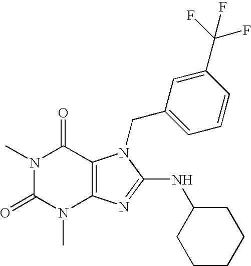 |
| 164 | 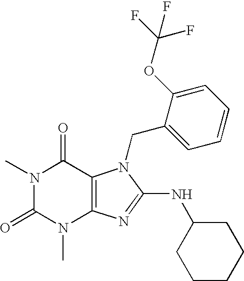 |
| 165 | 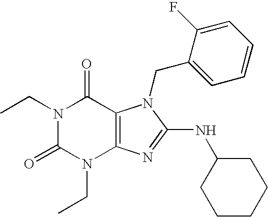 |
| 166 | 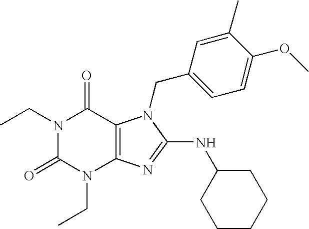 |
| 167 | 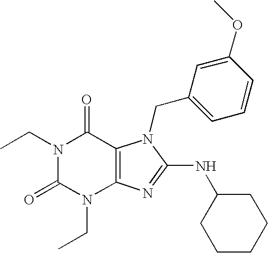 |
| 168 | 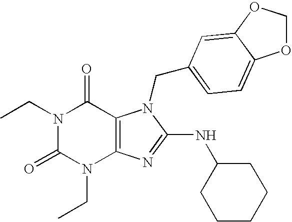 |
| 169 | 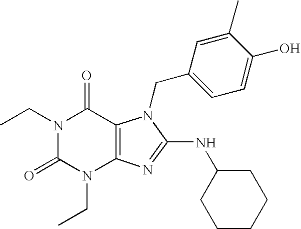 |
| 170 | 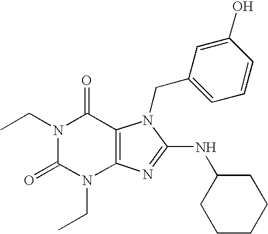 |
| 171 | 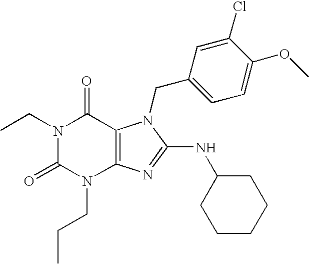 |
| 172 | 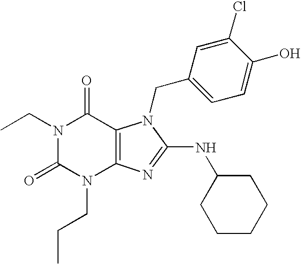 |
| 173 | 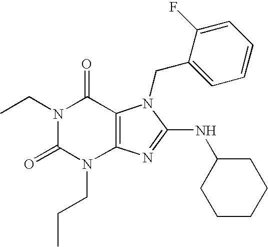 |
| 174 | 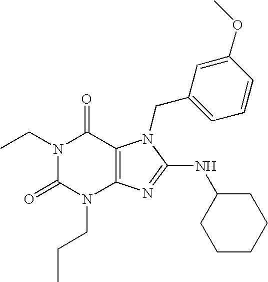 |
| 175 | 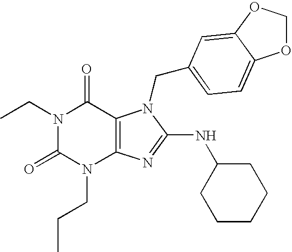 |
| 176 | 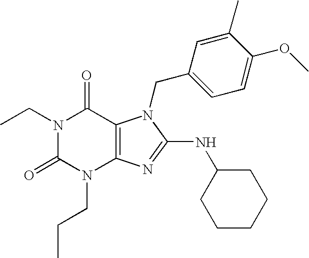 |
| 177 | 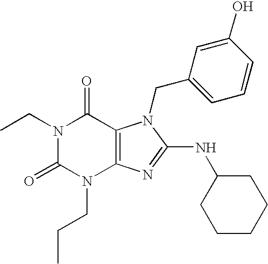 |
| 178 | 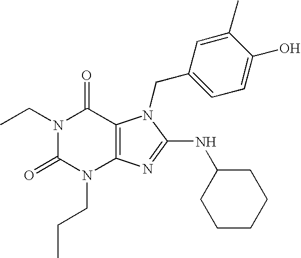 |
| 179 | 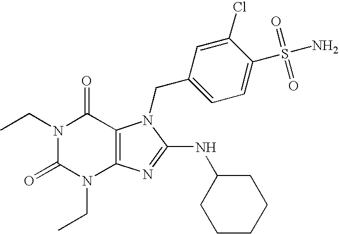 |
| 180 | 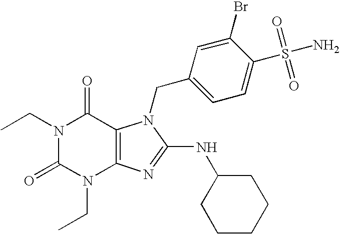 |
| 181 | 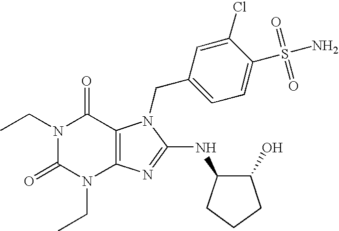 |
| 182 | 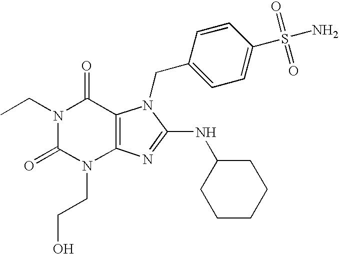 |
| 183 | 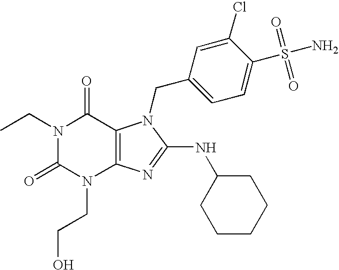 |
| 184 | 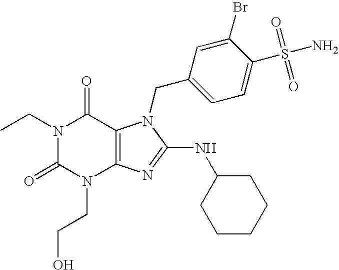 |
| 185 | 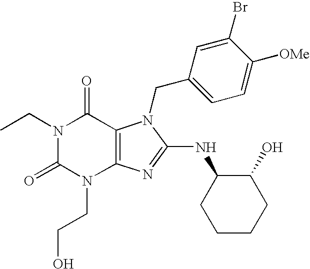 |
| 186 | 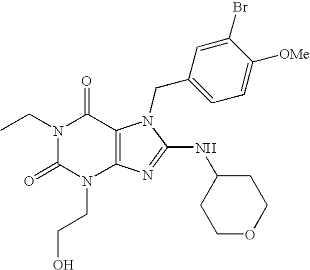 |
| 187 | 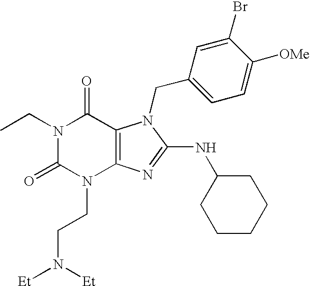 |
| 188 | 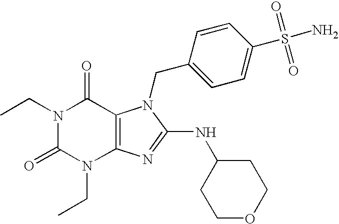 |
| 189 | 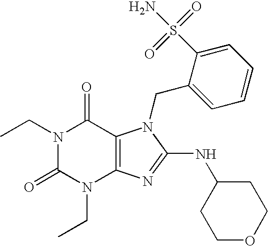 |
| 190 | 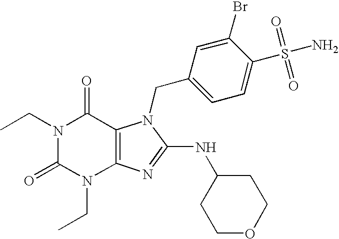 |
| 191 | 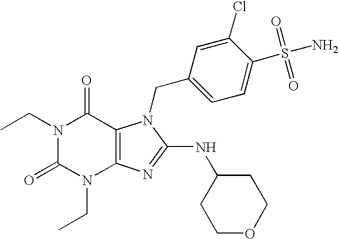 |
| 192 | 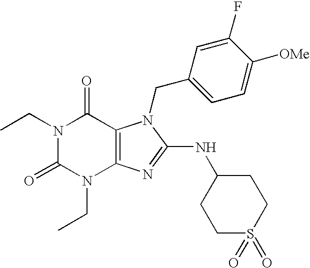 |
| 193 | 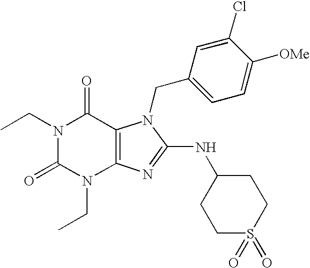 |
| 194 | 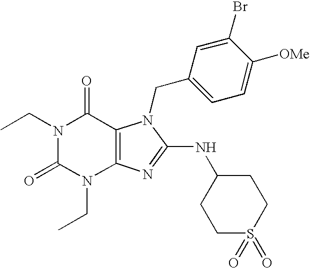 |
| 195 | 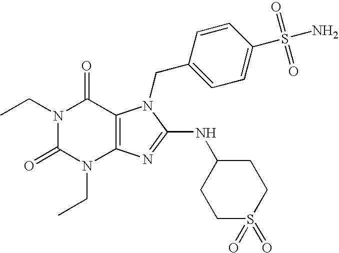 |
| 196 | 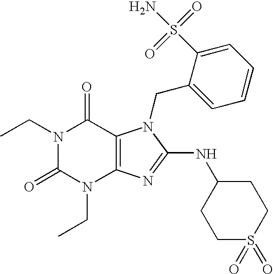 |
| 197 | 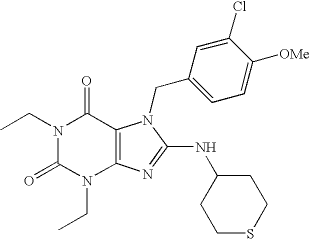 |
| 198 | 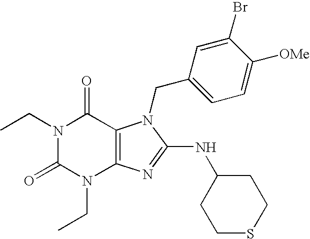 |
| 199 | 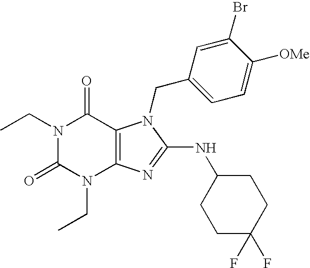 |

8. The method according to claim 1, wherein said compound is selected from the group consisting of:

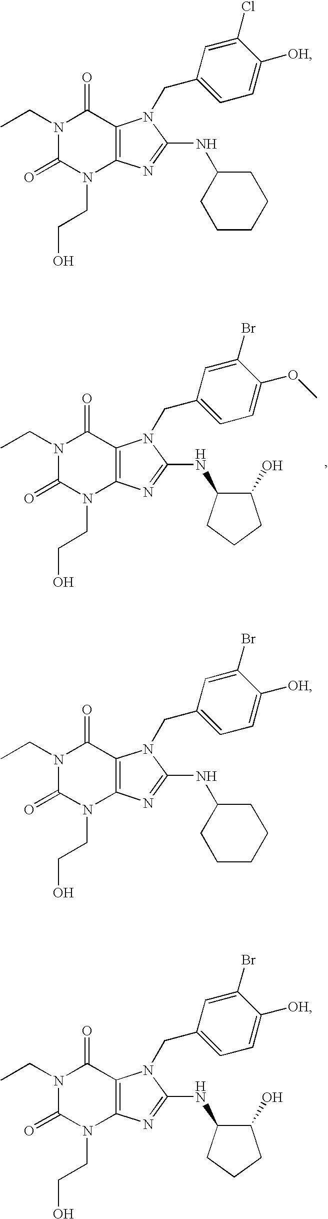

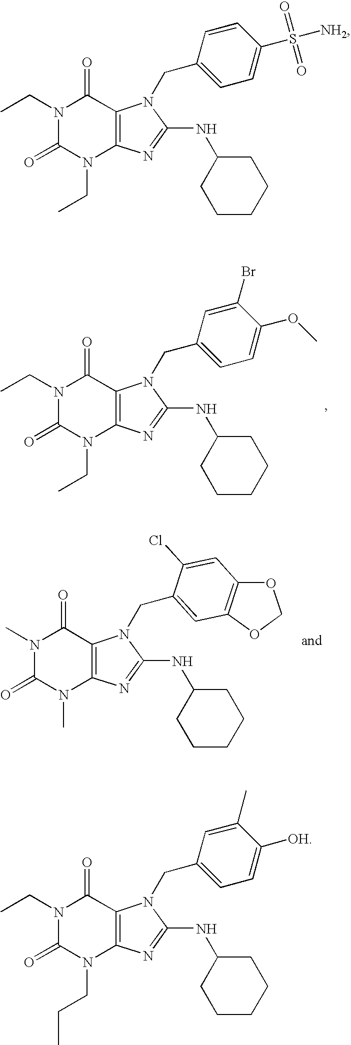

9. The method according to claim 1, wherein said compound is:

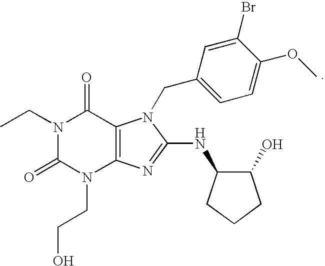

10. The method according to claim 9 further comprising administering to the patient an effective amount of at least one therapeutic agent selected from the group consisting of prostanoids, α-adrenergic receptor, dopamine receptor agonists, melanocortin receptor agonists, endothelin receptor antagonists, endothelin converting enzyme inhibitors, angiotensin II receptor antagonists, angiotensin converting enzyme inhibitors, neutral metalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5.

11. The method according to claim 9 further comprising administering to the patient an effective amount of at least one ET_{A} receptor antagonist selected from the group consisting of bosentan, atrasentan, ambrisentan, darusentan, sitaxsentan, ABT-627, TBC-3711, CI-1034, SPP-301, SB-234551, ZD-4054, BQ-123 and BE-18257B.

12. The method according to claim 9 further comprising administering to the patient an effective amount of sitaxsentan.

13. A pharmaceutical composition comprising a PDE V inhibitor compound, an ET_{A} receptor antagonist, and a pharmaceutically acceptable carrier.

14. The pharmaceutical composition according to claim 13, wherein said PDE V inhibitor compound is selected from the group consisting of those compounds listed in Tables I and II:
TABLE I

| Compound | |
|---|---|
| No. | Structure |
| 10 | 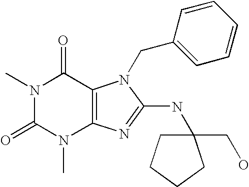 |
| 11 | 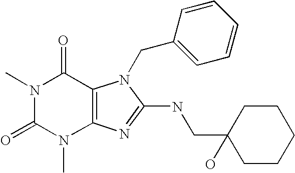 |
| 12 | 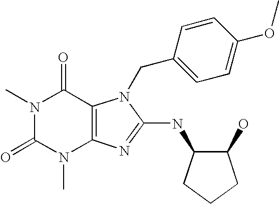 |
| 13 | 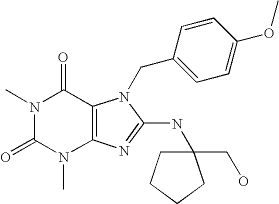 |
| 14 | 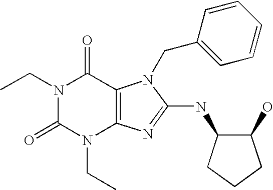 |
| 15 | 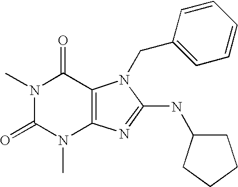 |
| 16 | 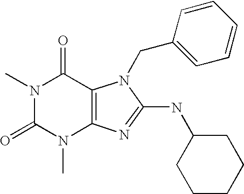 |
| 17 | 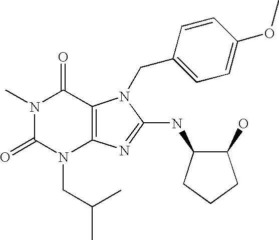 |
| 18 | 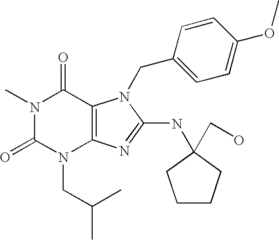 |
| 19 | 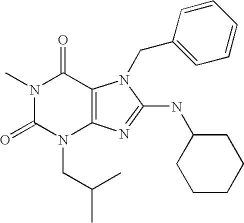 |
| 20 | 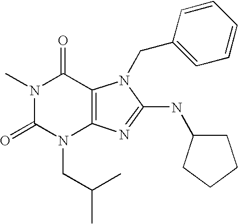 |
| 21 | 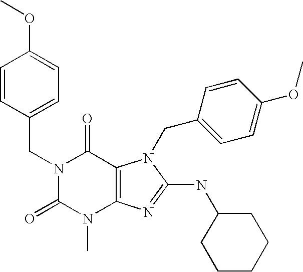 |
| 22 | 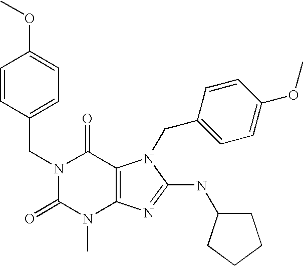 |
| 23 | 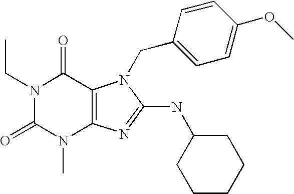 |
| 24 | 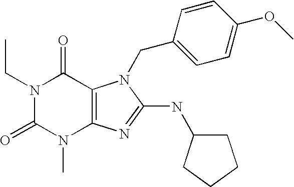 |
| 25 | 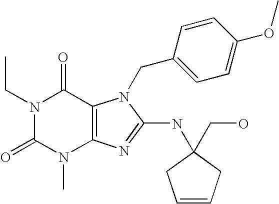 |
| 28 | 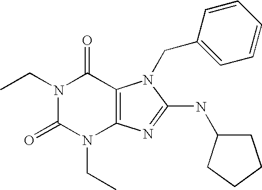 |
| 29 | 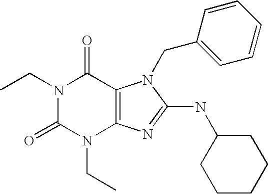 |
| 30 | 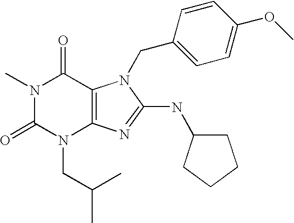 |
| 31 | 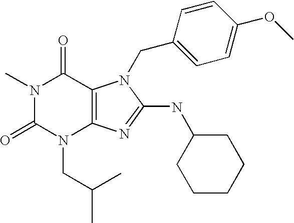 |
| 32 | 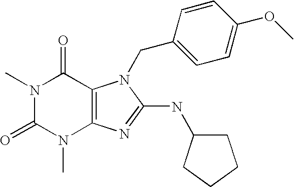 |
| 33 | 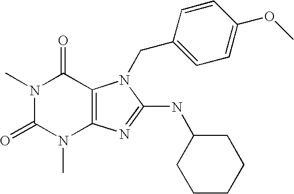 |
| 34 | 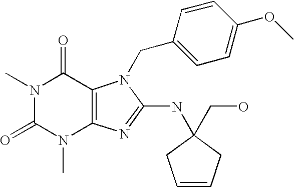 |
| 35 | 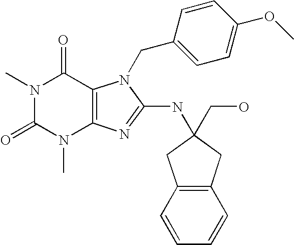 |
| 36 | 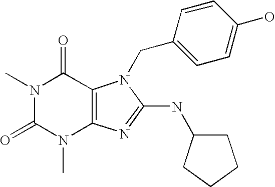 |
| 37 | 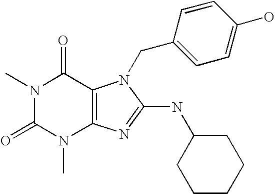 |
| 38 | 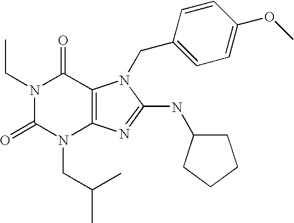 |
| 39 | 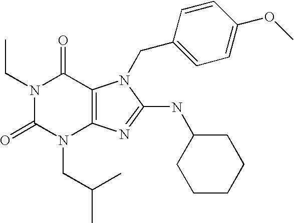 |
| 40 | 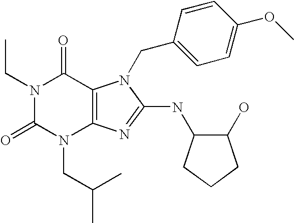 |
| 41 | 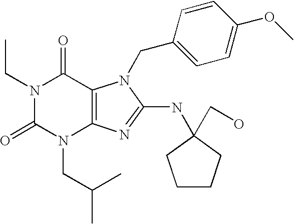 |
| 42 | 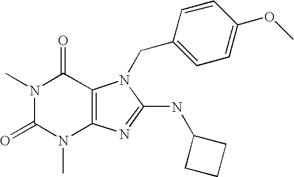 |
| 43 | 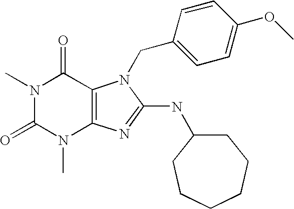 |
| 44 | 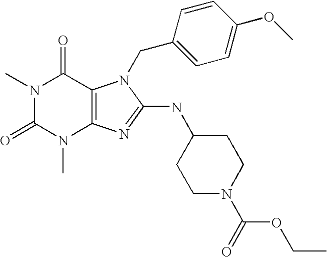 |
| 47 | 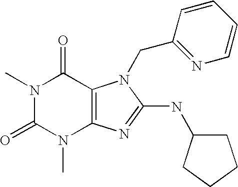 |
| 48 | 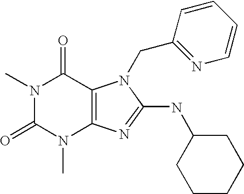 |
| 49 | 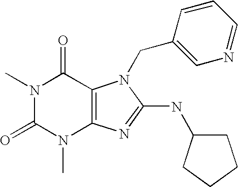 |
| 50 | 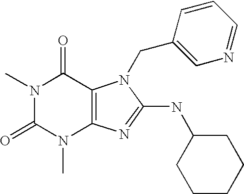 |
| 51 | 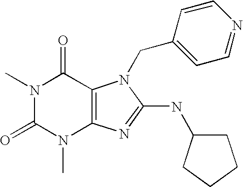 |
| 52 | 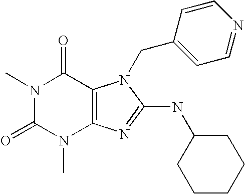 |
| 53 | 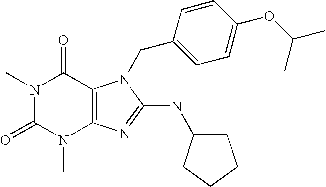 |
| 54 | 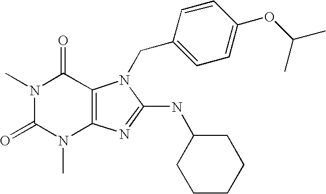 |
| 55 | 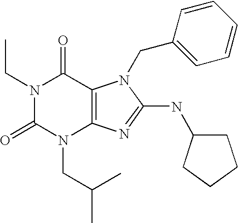 |
| 56 | 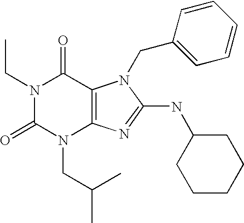 |
| 57 | 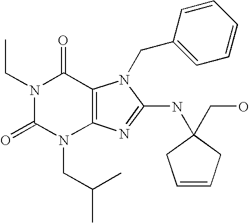 |
| 58 | 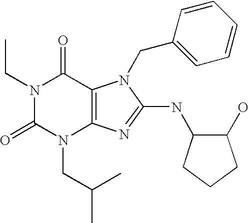 |
| 59 | 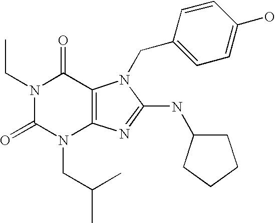 |
| 60 | 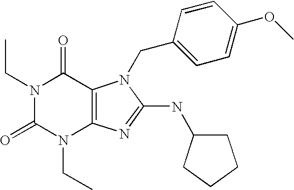 |
| 61 | 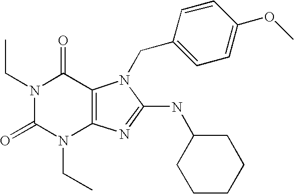 |
| 62 | 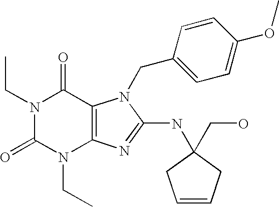 |
| 63 | 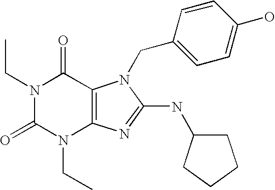 |
| 64 | 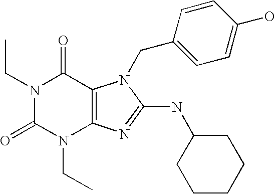 |
| 65 | 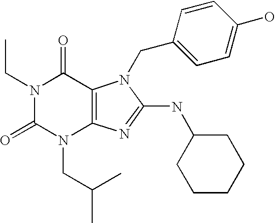 |
| 66 | 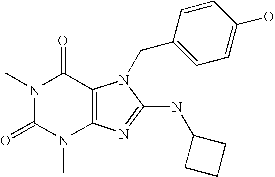 |
| 67 | 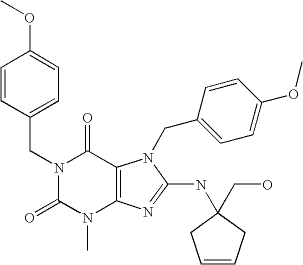 |
| 68 | 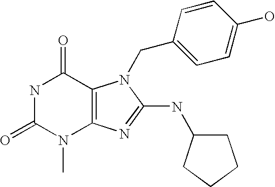 |
| 69 | 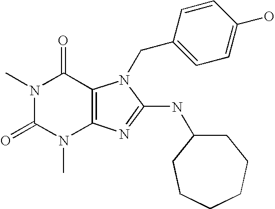 |
| 70 | 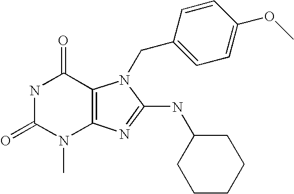 |
| 71 | 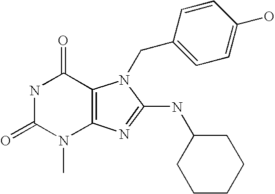 |
| 72 | 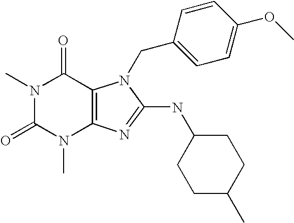 |
| 73 | 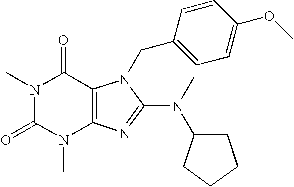 |
| 74 | 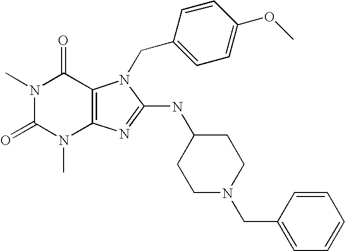 |
| 75 | 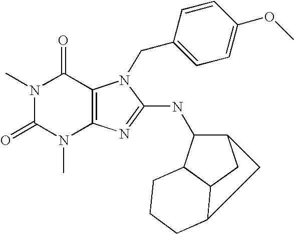 |
| 76 | 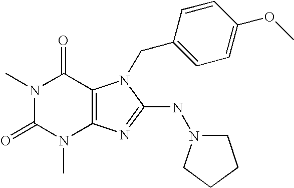 |
| 77 | 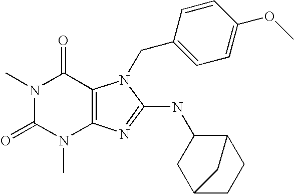 |
| 78 | 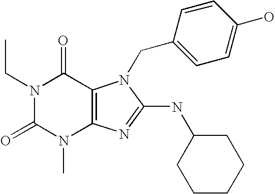 |
| 79 | 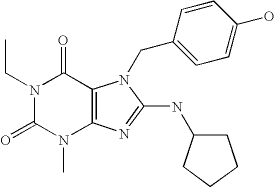 |
| 80 | 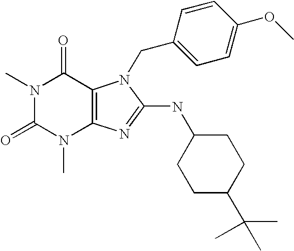 |
| 81 | 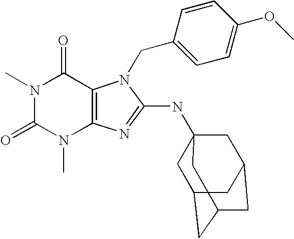 |
| 82 | 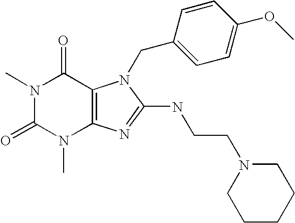 |
| 83 | 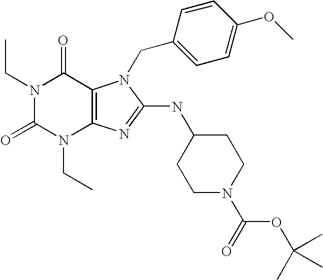 |
| 84 | 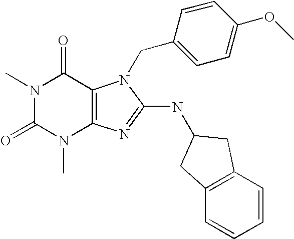 |
| 85 | 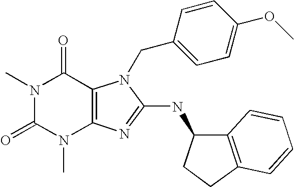 |
| 86 | 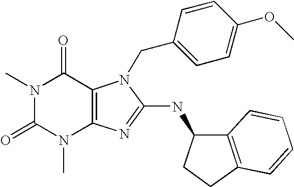 |
| 87 | 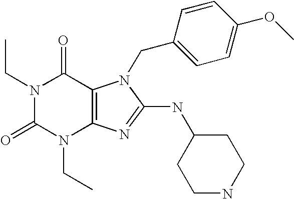 |
| 88 | 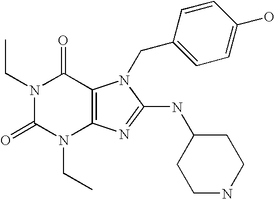 |
| 89 | 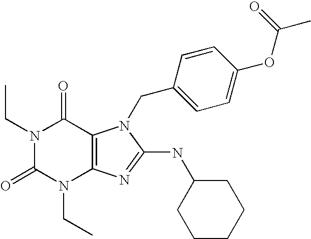 |
| 90 | 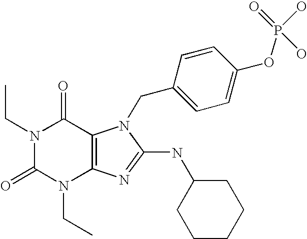 |
| 91 | 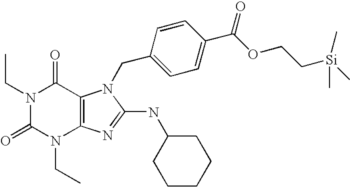 |
| 92 | 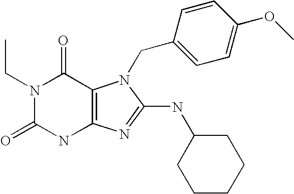 |
| 93 | 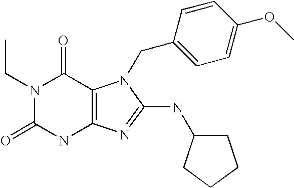 |
| 94 | 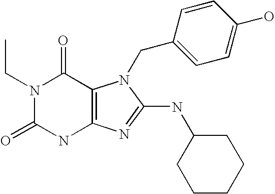 |
| 95 | 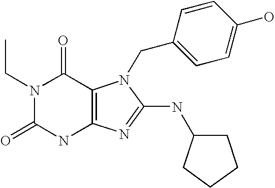 |
| 96 | 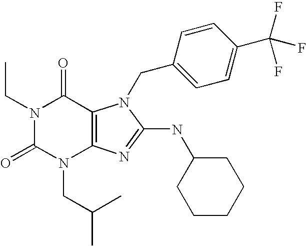 |
| 97 | 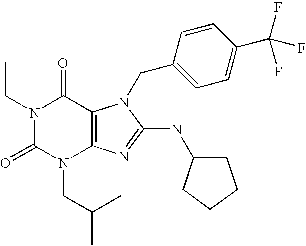 |
| 98 | 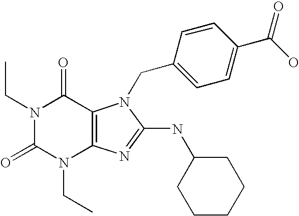 |
| 99 | 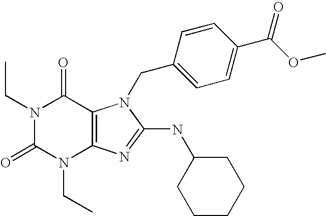 |
| 100 | 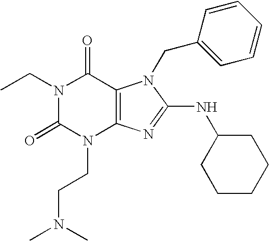 |
| 101 | 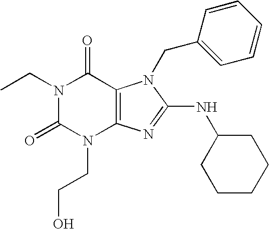 |
| 102 | 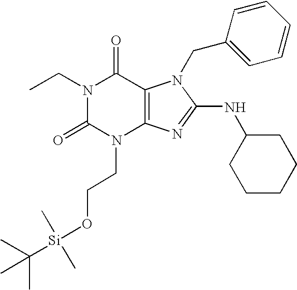 |
| 103 | 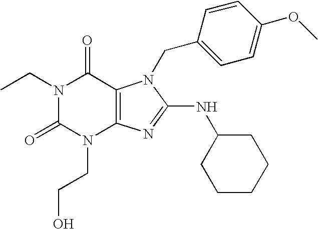 |
| 104 | 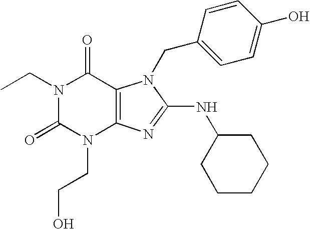 |
| 105 | 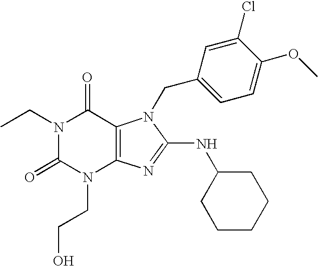 |
| 106 | 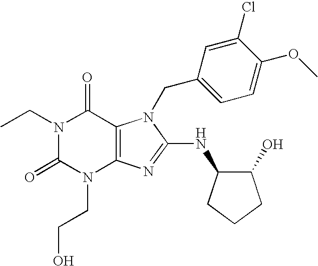 |
| 107 | 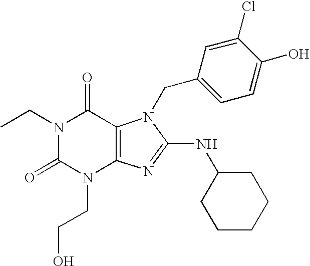 |
| 108 | 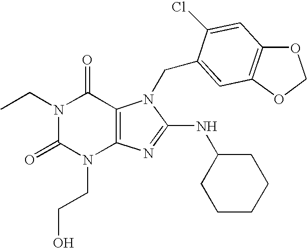 |
| 109 | 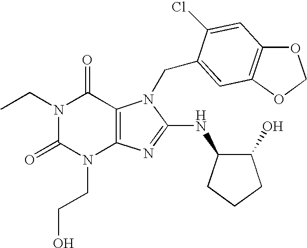 |
| 110 | 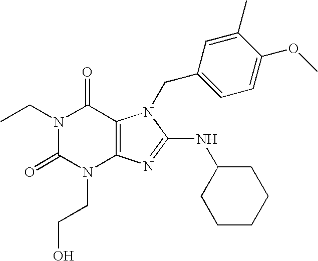 |
| 111 | 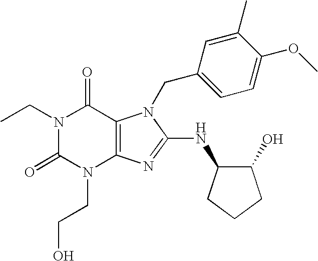 |
| 112 | 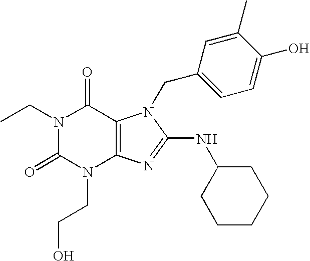 |
| 113 | 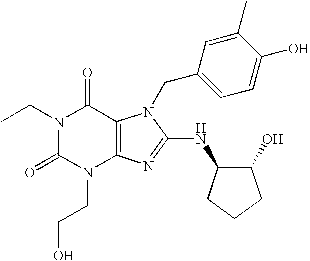 |
| 114 | 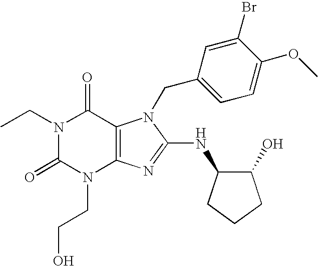 |
| 115 | 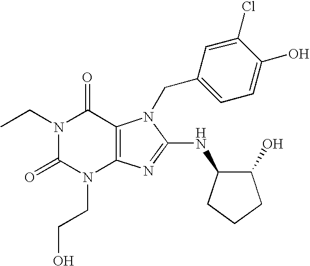 |
| 116 | 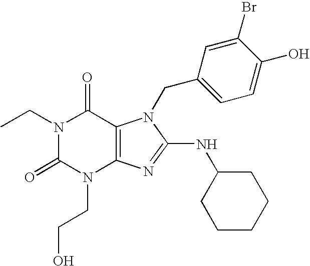 |
| 117 | 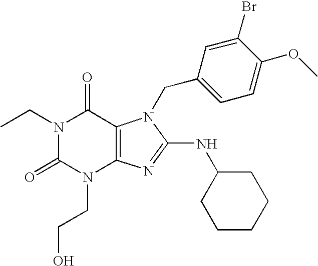 |
| 118 | 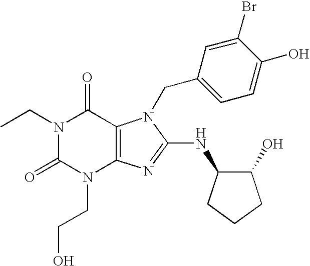 |
| 119 | 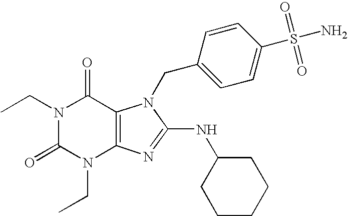 |
| 120 | 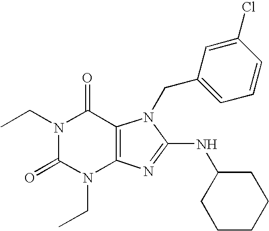 |
| 121 | 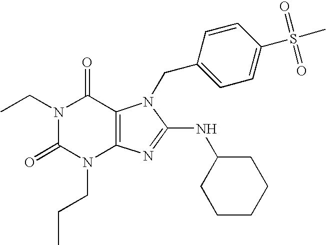 |
| 122 | 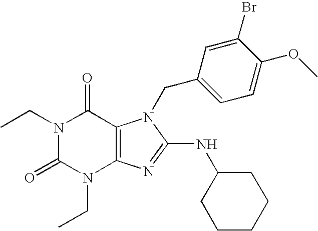 |
| 123 | 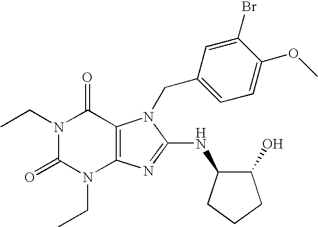 |
| 124 | 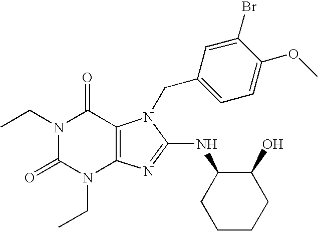 |
| 125 | 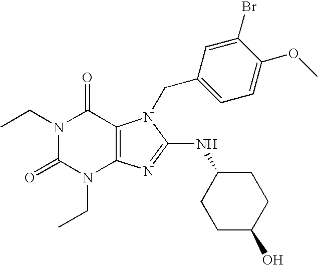 |
| 126 | 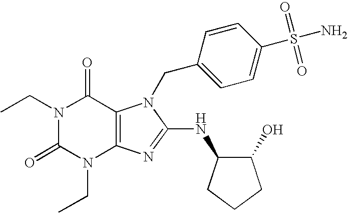 |
| 127 | 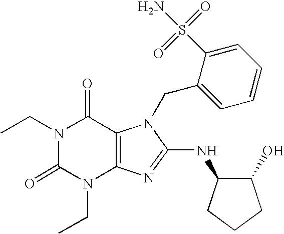 |
| 128 | 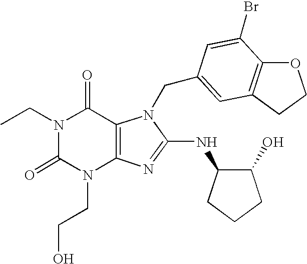 |
| 129 | 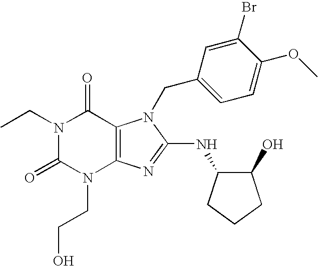 |
| 130 | no structure |
| 131 | 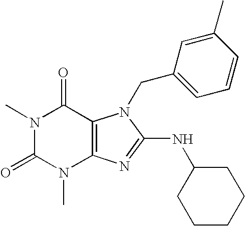 |
| 132 | 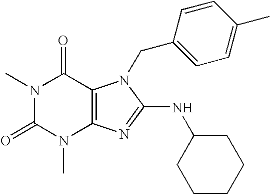 |
| 133 | 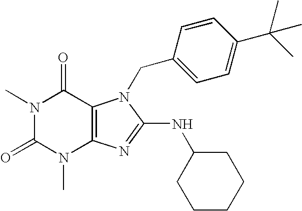 |
| 134 | 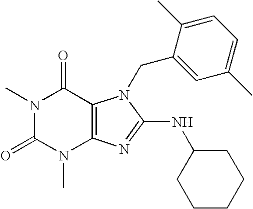 |
| 135 | 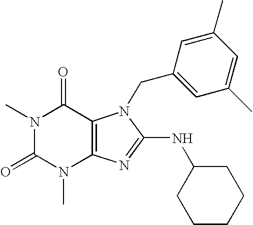 |
| 136 | 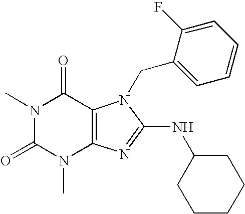 |
| 137 | 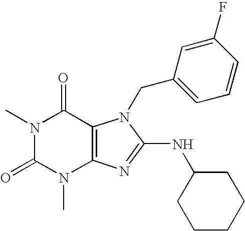 |
| 138 | 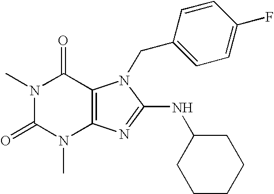 |
| 139 | 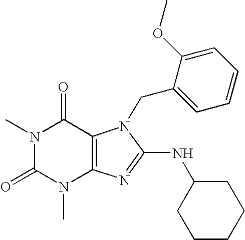 |
| 140 | 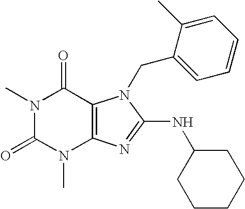 |
| 141 | 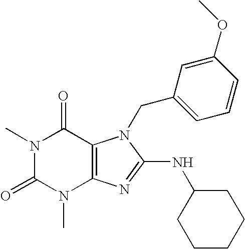 |
| 142 | 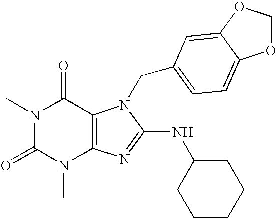 |
| 143 | 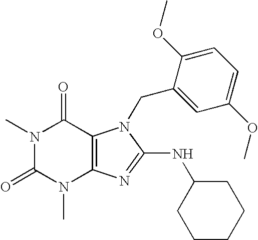 |
| 144 | 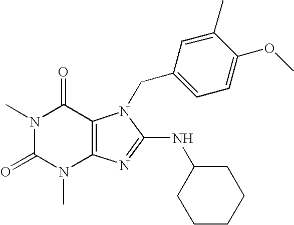 |
| 145 | 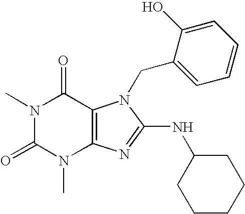 |
| 146 | 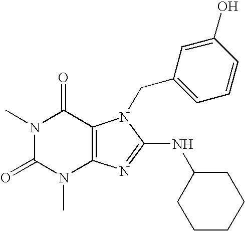 |
| 147 | 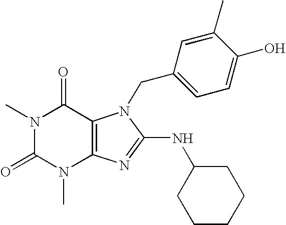 |
| 148 | 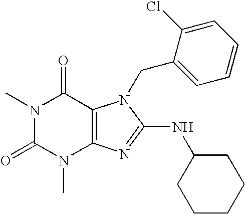 |
| 149 | 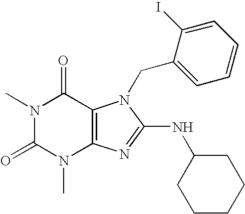 |
| 150 | 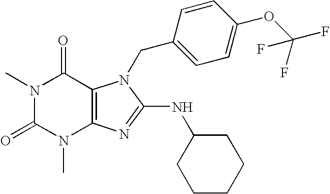 |
| 151 | 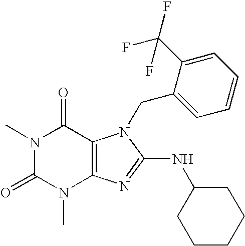 |
| 152 | 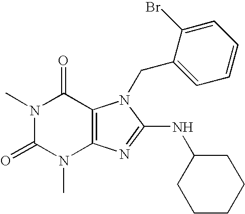 |
| 153 | 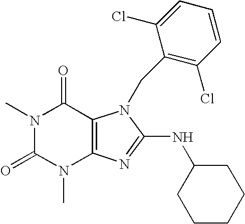 |
| 154 | 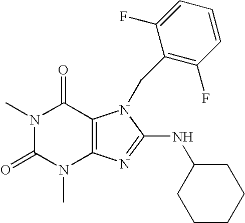 |
| 155 | 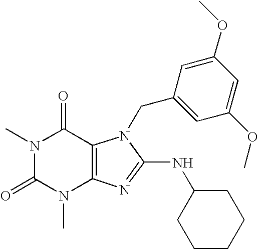 |
| 156 | 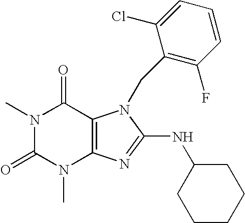 |
| 157 | 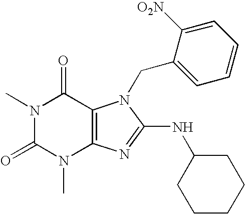 |
| 158 | 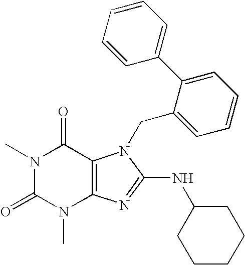 |
| 159 | 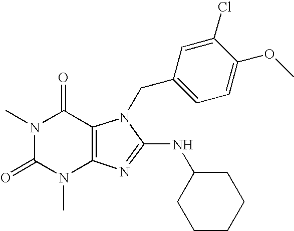 |
| 160 | 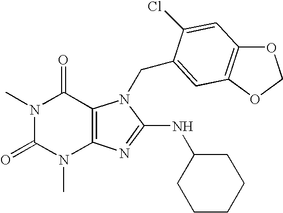 |
| 161 | 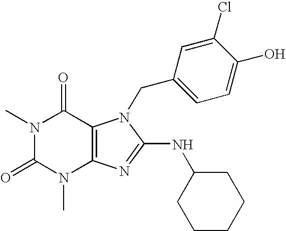 |
| 162 | 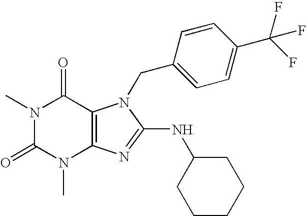 |
| 163 | 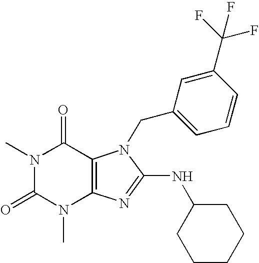 |
| 164 | 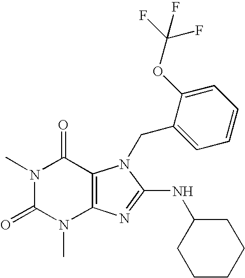 |
| 165 | 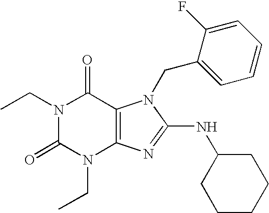 |
| 166 | 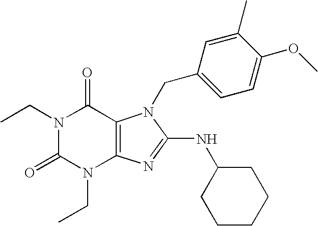 |
| 167 | 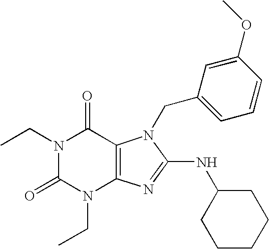 |
| 168 | 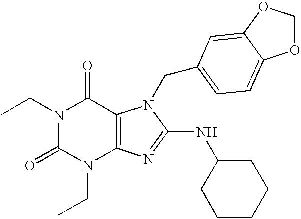 |
| 169 | 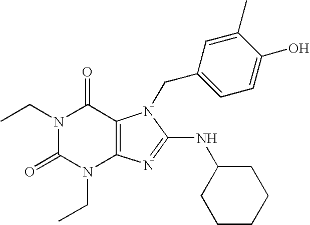 |
| 170 | 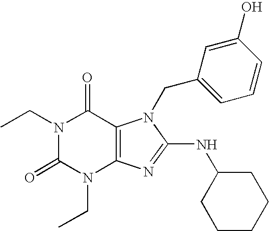 |
| 171 | 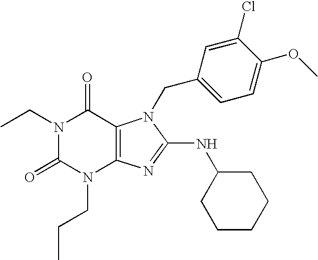 |
| 172 | 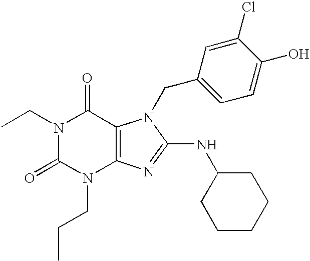 |
| 173 | 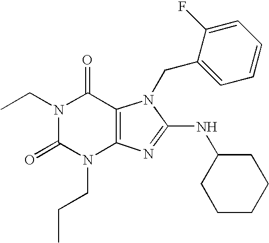 |
| 174 | 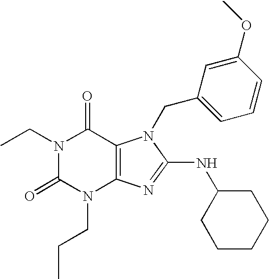 |
| 175 | 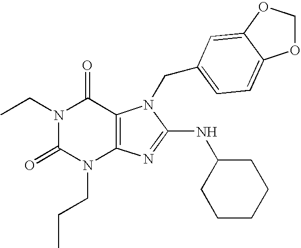 |
| 176 | 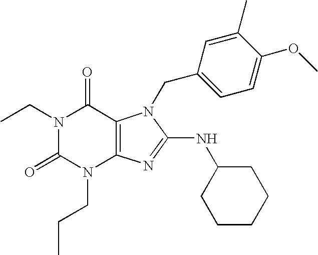 |
| 177 | 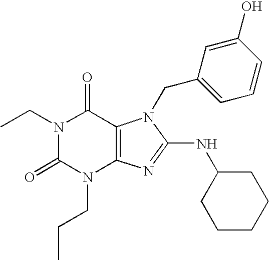 |
| 178 | 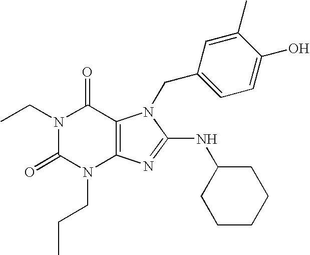 |
| 179 | 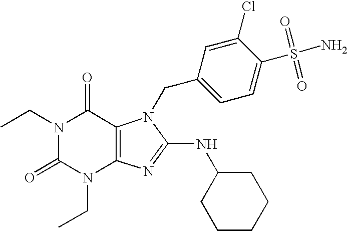 |
| 180 | 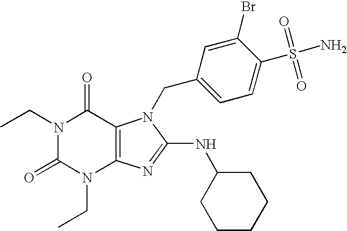 |
| 181 | 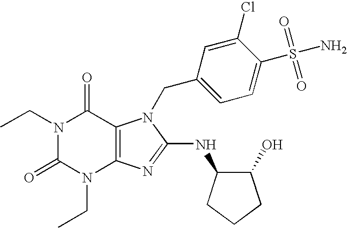 |
| 182 | 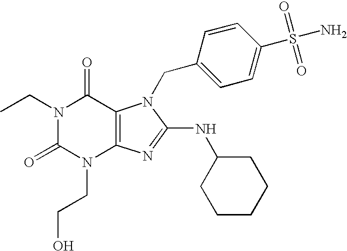 |
| 183 | 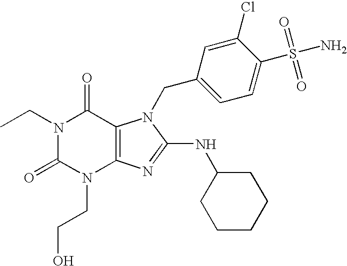 |
| 184 | 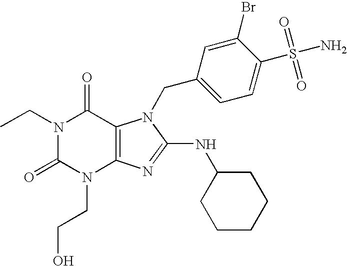 |
| 185 | 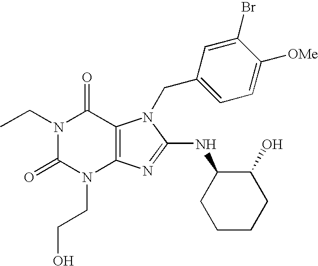 |
| 186 | 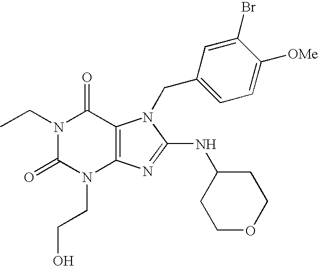 |
| 187 | 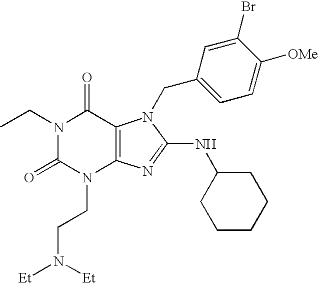 |
| 188 | 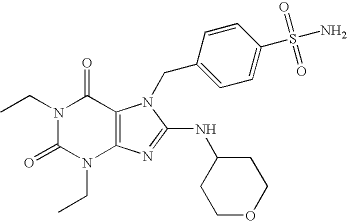 |
| 189 | 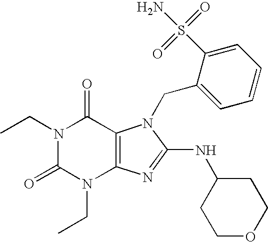 |
| 190 | 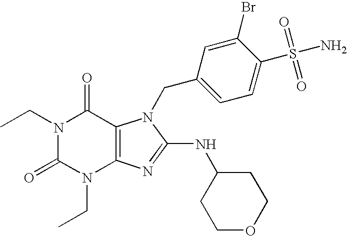 |
| 191 | 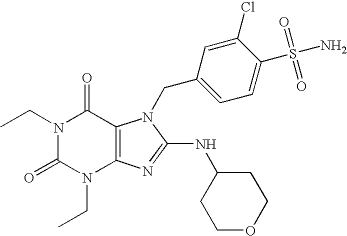 |
| 192 | 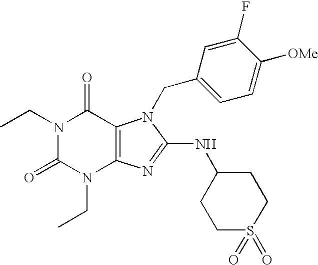 |
| 193 | 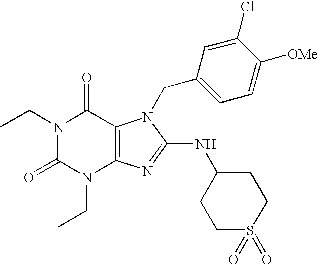 |
| 194 | 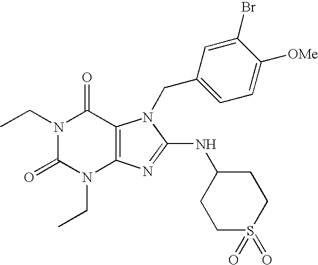 |
| 195 | 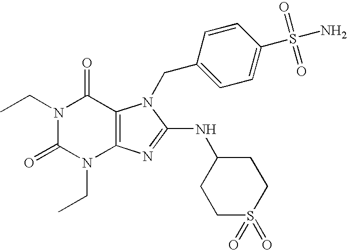 |
| 196 | 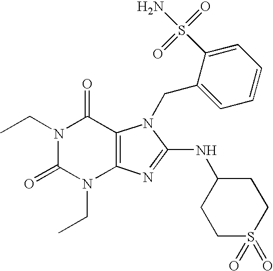 |
| 197 | 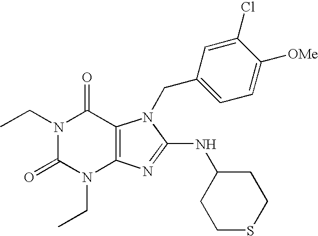 |
| 198 | 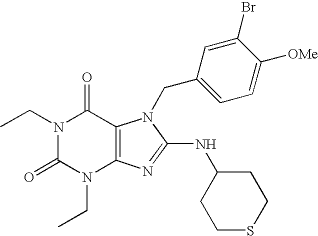 |
| 199 | 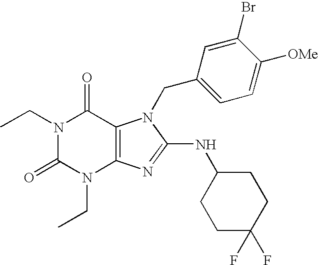 |

15. The pharmaceutical composition according to claim 13, wherein said PDE V inhibitor compound is selected from the group consisting of:

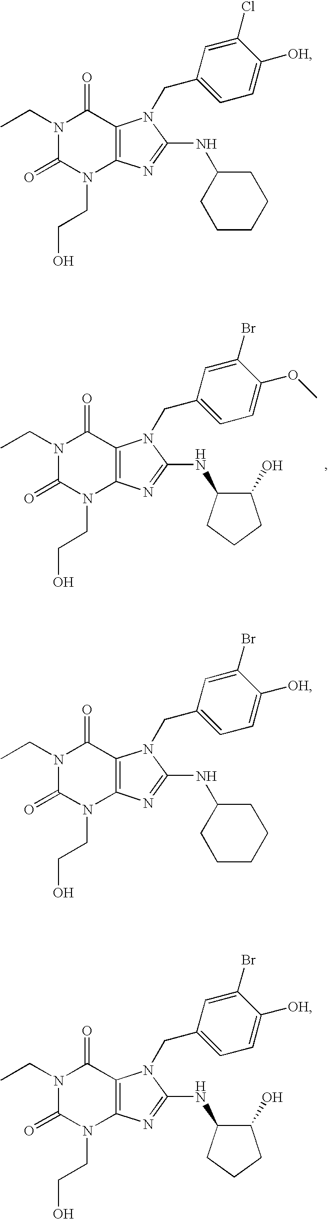

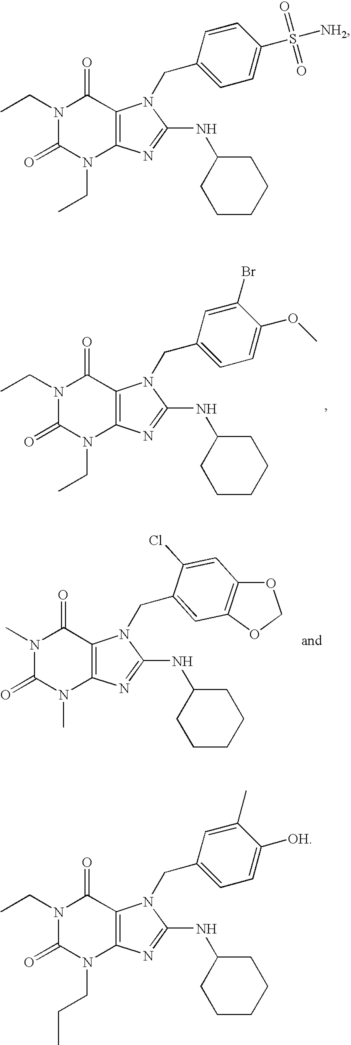

16. The pharmaceutical composition according to claim 13, wherein said PDE V inhibitor compound is

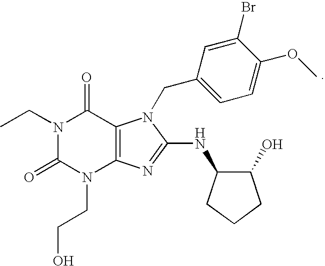

17. The pharmaceutical composition according to claim 16, wherein said ET_{A} receptor antagonist is sitaxsentan.
